(19) United States
(12) Patent Application Publication
(10) Pub. No.: US 2026/0183387 A1
(43) Pub. Date: Jul. 2, 2026

(54) METHODS OF TREATING HSV ORAL INFECTION OR ENCEPHALITIS

(71) Applicant: The trustees of the university of Pennsylvania, Philadelphia, PA (US)

(72) Inventors: Harvey FRIEDMAN, Philadelphia, PA (US); Drew Weissman, Wynnewood, PA (US); Sita Awasthi, Bala Cynwyd, PA (US)

(73) Assignee: The trustees of the university of Pennsylvania, Philadephia, PA (US)

(21) Appl. No.: 19/405,511

(22) Filed: Dec. 2, 2025

Related U.S. Application Data
(63) Continuation of application No. 18/470,325, filed on Sep. 19, 2023.
Application No. 18/470,325 is a continuation of application No. 17/349,929, filed on Jun. 17, 2021, now Pat. No. 11,793,872.
Application No. 17/349,929 is a continuation of application No. 16/640,008, now Pat. No. 11,141,478.
(63) Continuation of application No. PCT/US24/32163, filed on Jun. 2, 2024.
(60) Provisional application No. 62/546,648, filed on Aug. 17, 2017.
(60) Provisional application No. 62/701,019, filed on Jul. 20, 2018.
(60) Provisional application No. 63/506,028, filed on Jun. 2, 2023.
(60) Provisional application No. 63/517,586, filed on Aug. 3, 2023.

(51) Int. Cl. A61K 39/245; A61K 9/00; A61K 39/00; A61P 31/22; C07K 14/005; C12N 7/00; C12N 15/11

(52) U.S. Cl. CPC A61K 39/245; A61K 9/0036; A61P 31/22; C07K 14/005; C12N 7/00; C12N 15/11; A61K 2039/53; A61K 2039/54; A61K 2039/57; C12N 2710/16622

(57) ABSTRACT

The present invention provides compositions for the prevention and treatment of genital herpes, comprising nucleoside modified mRNAs that encode herpes simplex virus (HSV) glycoproteins, including those involved in virus entry and immune evasion, and methods of use thereof.

### CROSS-REFERENCE TO RELATED APPLICATIONS

This application is a continuation of U.S. application Ser. No. 18/470,325, filed on Sep. 19, 2023, which is a continuation of U.S. patent application Ser. No. 17/349,929 filed Jun. 17, 2021 (now U.S. Pat. No. 11,793,872). The latter is a Continuation of U.S. patent application Ser. No. 16/640,008, filed Feb. 18, 2020 (now U.S. Pat. No. 11,141,478), which in turn is a National Phase of PCT International Application No. PCT/IB2018/056210 filed Aug. 17, 2018, claiming the benefit of U.S. Provisional Application No. 62/701,019, filed Jul. 20, 2018, and U.S. Provisional Application No. 62/546,648, filed Aug. 17, 2017.

This application is also a continuation of PCT International Application No. PCT/US24/32163, filed on Jun. 2, 2024, which claims priority to U.S. Provisional Application No. 63/517,586, filed on Aug. 3, 2023, and U.S. Provisional Application No. 63/506,028, filed on Jun. 2, 2023. Each of the foregoing applications is incorporated herein by reference in its entirety.

### STATEMENT REGARDING FEDERALLY SPONSORED RESEARCH OR DEVELOPMENT

This invention was made with government support under AI104854 awarded by the National Institutes of Health. The government has certain rights in the invention.

### SEQUENCE LISTING STATEMENT

The instant application contains a Sequence Listing that has been submitted electronically in XML format and is hereby incorporated by reference in its entirety. The XML copy, created on Dec. 2, 2025, is named P-572137-US4-SL.xml and is 43,685 bytes in size.

### FIELD OF INVENTION

The present invention provides compositions for the prevention and treatment of genital herpes, comprising nucleoside modified mRNAs that encode herpes simplex virus (HSV) glycoproteins, including those involved in virus entry and immune evasion, and methods of use thereof.

### BACKGROUND OF THE INVENTION

A genital herpes vaccine is urgently needed to prevent pain and suffering, reduce the incidence of neonatal herpes, and decrease the risk of HIV acquisition and transmission that accompanies genital infection. Approximately a half-billion people worldwide are infected with herpes simplex virus type 2 (HSV-2), the virus that causes genital herpes. In some individuals, infection results in painful, recurrent genital ulcers, while in others, the infection remains quiescent. In both settings, infected individuals may transmit virus to their intimate partners. Genital herpes increases the risk that an infected person will acquire HIV if exposed during sexual intercourse. A vaccine for genital herpes is urgently needed, yet none is available.

Chiron Corp. evaluated a prophylactic vaccine containing two HSV-2 glycoproteins involved in virus entry, glycoproteins B (gB2) and D (gD2) given with MF59 as adjuvant. The vaccine did not protect seronegative partners from HSV-2 infection, although it delayed onset of infection over the first 5 months after immunization. GlaxoSmithKline (GSK) assessed a prophylactic vaccine using gD2 antigen with monophosphoryl lipid A (MPL) and alum as adjuvants. Overall, no protection against genital lesions was detected, although significant protection was noted in a subgroup of HSV-1 and HSV-2 doubly seronegative women. A follow-up trial was performed in doubly seronegative women that showed no overall protection against genital herpes; however, the vaccine was efficacious against HSV-1. This result was noteworthy because HSV-1 accounted for 60% of genital herpes infections in the control group. These studies indicate that targeting a vaccine to block HSV-2 entry is not sufficient.

HSV-1 and HSV-2 gC are immune evasion molecules that function as regulators of the complement cascade. During complement activation, C3, the most abundant complement protein, is cleaved to C3b, which activates the membrane attack complex leading to virus neutralization and lysis of infected cells. C3b stimulates B- and T-cell responses and serves as a link between innate and acquired immunity. HSV-1 and HSV-2 gC bind C3b to inhibit activities mediated by C3b. Immunization with gC1 and gC2 produces antibodies that bind to the glycoprotein and block its immune evasion functions.

HSV-1 and HSV-2 glycoprotein E (gE) function as immune evasion molecules by binding the Fc domain of an IgG molecule that is bound by its F(ab′)2 domain to its target. A vaccine containing gE2 subunit antigen produces antibodies that bind to gE2 and block its immune evasion functions. HSV-2 gC2 and gE2 perform activities similar to mammalian complement and IgG Fc regulatory proteins, yet share no sequence homology with mammalian receptors, which suggests virtually no risk that immunization will induce autoimmunity.

Previous work from our lab examined vaccines containing gC, gD and gE and found that such vaccines provide protection against HSV infection. However, it is not known if mRNA vaccines encoding HSV gC, gD and gE would be effective in protecting against HSV infection.

Using nucleic acids as vaccines has multiple advantages. Nucleic acid vaccines can induce both humoral and cellular immune responses; have low effective dosages; are simple to manipulate; avail rapid testing; are cost-effective and reproducible in large scale production and isolation; can be produced at high frequency and are easily isolated; are more temperature-stable than conventional vaccines; have a long shelf-life; are easy to store and transport; and are unlikely to require a cold chain (Shedlock & Weiner, J Leukocyte Biol. Vol 68, 2000).

In principle, either exogenous DNA or RNA can express proteins in the mammalian body. Whether or not similar immune activity can be produced with both DNA and mRNA expressed proteins is uncertain. Conventional wisdom is that DNA is superior for the creation of vaccines and gene therapy due to its stability and ease of use.

DNA has been used in vaccines with success. DNA is fairly stable and unreactive and can be stored long term. However, DNA is self-replicating and can be easily damaged by ultra-violet radiation. DNA based vaccines may also raise safety concerns due to possible insertion of DNA into the genome, possible interruption of genes and formation of anti-DNA antibodies.

RNA vaccines exhibit important safety features. RNA is more reactive than DNA and less stable but is resistant to ultra-violet radiation. mRNA does not integrate into the host chromosomes. The delivery of mRNA results in faster expression of the antigen of interest and requires fewer copies for expression. mRNA expression is transient, which adds to its safety. mRNA is more effective than DNA for protein production in post mitotic and non-dividing cells because DNA requires translocation through the nuclear member and plasma membrane, while mRNA requires translocation only through the plasma membrane. mRNA is not only a template for translation, but also acts as a ligand for toll-like receptors and is nuclease sensitive; therefore it presents less concern for horizontal transmission.

In addition, RNA vaccines elegantly integrate adjuvanticity and antigen expression, thereby mimicking relevant aspects of viral infections. This increases their efficacy compared to inactivated vaccines that require the use of adjuvants, simplifying handling and production. RNA can address a range of dedicated immunologic pattern recognition receptors, including toll-like receptors 3, 7, and 8, RIG-I, MDA5, PKR, and others that may act synergistically and serve to enhance the induction of antigen-specific adaptive B and T cell responses. Importantly, by antigen synthesis in transfected host cells, mRNA vaccines directly introduce antigen into cellular antigen processing and presentation pathways, granting access to MHC molecules and triggering T cell responses, irrespective of the hosts MHC haplotype. This enables the induction of polyclonal T cell responses that may act synergistically with other immune responses, including B cells. Also, endogenous production of antigen ensures faithful posttranslational modification (e.g. proteolytic processing, glycosylation, etc.) that may positively impact immunogenicity.

### SUMMARY OF THE INVENTION

In one embodiment, the present invention provides compositions comprising one or more nucleoside modified mRNAs, wherein each of said nucleoside modified mRNAs encodes a Herpes Simplex Virus (HSV) glycoprotein or immunogenic fragment thereof, and wherein said nucleoside modified mRNA comprises one or more pseudouridine residues.

In another embodiment, the present invention provides compositions comprising one or more nucleoside modified mRNAs, wherein each of said modified mRNAs encodes a Herpes Simplex Virus (HSV) glycoprotein or immunogenic fragment thereof, and wherein said nucleoside modified mRNA comprises 1-methylpseudouridine, wherein said pseudouridine residues comprise m¹acp³Ψ (1-methyl-3-(3-amino-5-carboxypropyl)pseudouridine, m¹Ψ (1-methylpseudouridine), Ψm (2′-O-methylpseudouridine, m⁵D (5-methyldihydrouridine), m³Ψ (3-methylpseudouridine), or any combination thereof.

In another embodiment, the present invention provides compositions comprising modified mRNAs comprising one or more pseudouridine residues, wherein each of said modified mRNAs encode an a) HSV glycoprotein D (gD) or an immunogenic fragment thereof, b) HSV glycoprotein C (gC) or an immunogenic fragment thereof, c) HSV glycoprotein E (gE) or an immunogenic fragment thereof, or any combination thereof.

In another embodiment, the present invention provides a method of treating a Herpes Simplex Virus (HSV) infection in a subject, the method comprising the step of administering to said subject a composition comprising one or more modified mRNAs, wherein each of said modified mRNAs encodes an HSV glycoprotein or immunogenic fragment thereof, and wherein said modified mRNA comprises pseudouridine residues.

In another embodiment, the present invention provides a method of inducing an immune response in a subject, comprising the step of administering to said subject a composition comprising one or more modified mRNAs, wherein each of said modified mRNAs encodes an HSV glycoprotein or immunogenic fragment thereof, and wherein said modified mRNA comprises pseudouridine residues.

In a further embodiment, the present invention provides a method of suppressing, inhibiting, or reducing the incidence of a Herpes Simplex Virus (HSV) infection in a subject, the method comprising the step of administering to said subject a composition comprising one or more modified mRNAs, wherein each of said modified mRNAs encodes an HSV glycoprotein or immunogenic fragment thereof, and wherein said modified mRNA comprises pseudouridine residues.

In yet another embodiment, the present invention provides a method of treating a Herpes Simplex Virus (HSV) infection in a subject, the method comprising the step of administering to said subject a composition comprising one or more modified mRNAs encoding a) an HSV glycoprotein D (gD) or an immunogenic fragment thereof, b) an HSV glycoprotein C (gC) or an immunogenic fragment thereof, c) an HSV glycoprotein E (gE) or an immunogenic fragment thereof, or any combination thereof.

In a yet a further embodiment, the present invention provides a method of suppressing, inhibiting, or reducing the incidence of a Herpes Simplex Virus (HSV) infection in a subject, the method comprising the step of administering to said subject a composition comprising one to three modified mRNAs, wherein each of said modified mRNAs encodes a) HSV glycoprotein D (gD) or an immunogenic fragment thereof, b) HSV glycoprotein C (gC) or an immunogenic fragment thereof, and c) HSV glycoprotein E (gE) or an immunogenic fragment thereof, or any combination thereof.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

FIGS. 1A-1C. Characterization of exemplary translational products of an immunogenic fragment of the ectodomain from HSV-2 gC (gC2)-, HSV-2 gD (gD2)- and HSV-2 gE (gE2)-modified RNA in Vero cells.

FIG. 1A. Western blot showing expression of an immunogenic fragment of gC2 from an exemplary modified RNA.

FIG. 1B. Western blot showing expression of an immunogenic fragment of gD2 from an exemplary modified RNA.

FIG. 1C. Western blot showing expression of an immunogenic fragment of gE2 from an exemplary modified RNA.

FIG. 2A. gC2 antibody (Ab) response as determined by antigen-specific ELISA in mice immunized with an exemplary gD2 RNA; or an exemplary combination of a gC2 RNA, a gD2 RNA and a gE2 RNA each given at a different intradermal site (Trivalent-I); or an exemplary combination of a gC2 RNA, a gD2 RNA and a gE2 RNA given in a single composition (Trivalent-C). I indicates first immunization; II indicates second immunization.

FIG. 2B. gD2 Ab response as determined by antigen-specific ELISA in mice immunized with an exemplary gD2 RNA; or an exemplary combination of a gC2 RNA, a gD2 RNA and a gE2 RNA each given at a different intradermal site (Trivalent-I); or an exemplary combination of a gC2 RNA, a gD2 RNA and a gE2 RNA given in a single composition (Trivalent-C). I indicates first immunization; II indicates second immunization.

FIG. 2C. gE2 Ab response as determined by antigen-specific ELISA in mice immunized with an exemplary gD2 RNA; or an exemplary combination of a gC2 RNA, a gD2 RNA and a gE2 RNA each given at a different intradermal site (Trivalent-I); or an exemplary combination of a gC2 RNA, gD2 RNA and gE2 RNA given in a single composition (Trivalent-C). I indicates first immunization; II indicates second immunization.

FIG. 3A. Antigen-specific IgG1 responses in mice vaccinated with exemplary RNA compositions. Antibodies were evaluated after the first and second immunization for IgG1 responses. I indicates first immunization; II indicates second immunization.

FIG. 3B. Antigen-specific IgG2a responses in mice vaccinated with exemplary RNA compositions. Antibodies were evaluated after the first and second immunization for IgG2a responses. I indicates first immunization; II indicates second immunization.

FIG. 4. Neutralizing antibody titers in RNA vaccinated mice. 50% endpoint neutralization titers of serum were obtained after the second immunization. Titers were performed using 10% human complement. Trivalent-I animals were immunized with an exemplary gC2/lipid nanoparticle (LNP), an exemplary gD2/LNP, and an exemplary gE2/LNP, each given at a different site. Trivalent-C animals were immunized with an exemplary gC2, an exemplary gD2 and an exemplary gE2 combined into a single LNP. P values comparing 50% endpoint neutralizing titers: Trivalent-I versus gD2, p=0.04; Trivalent-C versus gD2, p=0.002; Trivalent-I versus Trivalent-C, p-0,026.

FIGS. 5A-5B. CD4⁺ T cell responses to an exemplary gC2, an exemplary gD2 and an exemplary gE2 RNA each administered at a different intradermal site. Splenocytes were stimulated with subunit antigen glycoproteins (FIG. 5A) or 15 amino acid peptides with 11 overlapping amino acids to stimulate HSV-2 specific T cell responses (FIG. 5B). * indicates p<0.05 (t test) comparing gC, gD or gE with PBS stimulated CD4⁺ T cells or DMSO stimulated CD4⁺ T cells. Error bars represent SD.

FIGS. 6A-6B. CD8⁺ T cell responses to an exemplary gC2, an exemplary gD2 and an exemplary gE2 RNA, each administered at a different intradermal site. Splenocytes were stimulated with subunit antigen glycoproteins (FIG. 6A) or 15 amino acid peptides with 11 overlapping amino acids to stimulate HSV-2 specific T cell responses (Figure B). * indicates p<0.05 comparing gE pool 2 with DMSO control. Error bars represent SD.

FIG. 7A. Survival in BALB/c mice immunized with exemplary RNA compositions twice at 28 day intervals and challenged intravaginally with HSV-2. Trivalent-I represents animals immunized with an exemplary gC2 RNA/LNP, an exemplary gD2 RNA/LNP, and an exemplary gE2 RNA/LNP each given at different intradermal sites. Trivalent-C represents animals immunized with an exemplary gC2 RNA, an exemplary gD2 RNA and an exemplary gE2 RNA combined into a single LNP for immunization.

FIG. 7B. Weight loss (−) or gain (+) and neurological signs in BALB/c mice immunized with exemplary RNA compositions twice at 28 day intervals and challenged intravaginally with HSV-2. Trivalent-I represents animals immunized with an exemplary gC2 RNA/LNP, an exemplary gD2 RNA/LNP, and an exemplary gE2 RNA/LNP each given at different intradermal sites. Trivalent-C represents animals immunized with an exemplary gC2 RNA, an exemplary gD2 RNA and an exemplary gE2 RNA combined into a single LNP for immunization.

FIGS. 8A-8B. Vaginal viral titers in mice vaccinated with exemplary RNA compositions after intravaginal challenge with HSV-2. Vaginal swab titers were obtained on Day 2 (FIG. 8A) and Day 4 (FIG. 8B) post-challenge. The dotted lines indicate the limit of detection of the assay at 7 PFU/ml.

FIG. 9. Genital disease in mice vaccinated with exemplary RNA compositions after HSV-2 vaginal challenge. Mice were immunized with poly C as a control, or with an exemplary gD2 RNA/LNP, trivalent at individual sites for each exemplary glycoprotein RNA (Trivalent-I) or trivalent with all three exemplary RNAs combined (Trivalent-C). Genital disease was scored for 28 days. All animals in the poly C group died by day 10. ***, indicates p<0.001 comparing poly C with the other 3 groups.

FIG. 10. HSV-2 DNA copies in dorsal root ganglia (DRG) in RNA vaccinated mice on day 4 post challenge. HSV-2 DNA in DRG was measured by qPCR. DRG from 4 to 5 animals per group were evaluated for HSV-2 DNA at 4 days post challenge. The bars represent the mean values per group.

FIG. 11A. The exemplary trivalent RNA-LNP vaccine induces a potent T follicular helper cell response in mice. BALB/c female mice were left un-immunized as naïve control animals or immunized intradermally twice at 28 day intervals with poly C RNA-LNP or an exemplary trivalent modified RNA-LNP. The poly C RNA controls received 10 μg Poly C RNA-LNP divided into 4 aliquots and administered at 4 separate sites. The exemplary trivalent modified RNA group received 10 μg of an exemplary gC2 RNA-LNP, 10 μg of an exemplary gD2 RNA-LNP, and 10 μg of an exemplary gE2 RNA-LNP each divided into 2 aliquots and each given at 2 sites. Two weeks after the second immunization, spleens were harvested from 5 animals per group and flow cytometry performed to detect T follicular helper (Tfh) cell responses (*p<0.05).

FIG. 11B. The exemplary trivalent RNA-LNP vaccine induces a potent germinal center B cell response in mice. BALB/c female mice were left un-immunized as naïve control animals or immunized intradermally twice at 28 day intervals with poly C RNA-LNP or an exemplary trivalent modified RNA-LNP. The poly C RNA controls received 10 μg Poly C RNA-LNP divided into 4 aliquots and administered at 4 separate sites. The trivalent modified RNA group received 10 μg of an exemplary gC2 RNA-LNP, 10 μg of an exemplary gD2 RNA-LNP, and 10 μg of an exemplary gE2 RNA-LNP each divided into 2 aliquots and each given at 2 sites. Two weeks after the second immunization, spleens were harvested from 5 animals per group and flow cytometry performed to detect germinal center B cell responses (*p<0.05).

FIGS. 12A-12C. Genital mucosa IgG antibody responses. BALB/c mice were immunized intradermally twice at 28 day intervals with 10 μg of poly C RNA-LNP, 10 μg of an exemplary gD2 RNA-LNP or 10 μg each of an exemplary gC2, an exemplary gD2, an exemplary gE2 trivalent modified RNA-LNP. The exemplary trivalent RNA was combined and administered as 10 μg of an exemplary gC2 RNA & 10 μg of an exemplary gD2 RNA & 10 μg of an exemplary gE2 RNA combined into LNP and divided into 4 aliquots and given at 4 sites. One month after the second immunization, 60 μl of media was introduced in the vaginal cavity and retrieved. IgG titers were determined at a 1:50 dilution of the vaginal wash fluids by ELISA to gC2 (FIG. 12A), gD2 (FIG. 12B), and gE2 (FIG. 12C) (n=10 mice in the poly C group, n=10 in the gD2 RNA group and n=25 in the trivalent RNA group; ***p<0.001; **p<0.01).

FIG. 13. The exemplary trivalent RNA-LNP vaccine produces antibodies that block gC2 binding to complement component C3b. BALB/c mice were left unimmunized as a source of non-immune IgG, or immunized intradermally with poly C RNA-LNP or an exemplary trivalent RNA-LNP. The poly C RNA controls received 10 μg poly C RNA-LNP divided into 4 aliquots and administered at 4 separate sites. The gD2 RNA group received 10 μg of an exemplary gD2 RNA-LNP administered as described for the poly C RNA-LNP. The trivalent modified RNA group received 10 μg of an exemplary gC2 RNA-LNP, 10 μg of an exemplary gD2 RNA-LNP, and 10 μg of an exemplary gE2 RNA-LNP combined into LNP and divided into 4 aliquots and given at 4 sites. There were 10 mice in each group. Sera from the 10 mice were pooled and IgG was purified. The IgG was evaluated at 12 μg/200 μl for its ability to block complement component C3b binding to gC2. (****p<0.0001).

FIGS. 14A-14F. The exemplary trivalent RNA vaccine provides outstanding protection in mice when the vaccine is administered intramuscularly. BALB/c mice were immunized intramuscularly with poly C RNA-LNP as a control (15/group) or with an exemplary trivalent RNA composition containing 10 μg each of an exemplary gC2, an exemplary gD2 and an exemplary gE2 RNA-LNP (20/group). FIG. 14A presents data on mouse survival; FIG. 14B presents data on weight loss; FIG. 14C presents data on genital disease. DRG were harvested from nine poly C animals at the time of euthanasia between days 7 and 12 post-infection or at the end of the experiment on day 28 in the trivalent RNA group. FIG. 14D presents data on HSV-2 DNA in DRG. FIG. 14E presents data on vaginal viral cultures on Day 2 and FIG. 14F presents data on vaginal viral cultures on Day 4. Difference between poly C and trivalent groups are significant, p<0.001 for FIGS. 14A-14F.

FIGS. 15A-15C. The exemplary trivalent RNA vaccine is highly efficacious in the guinea pig genital infection model. Hartley Strain female guinea pigs were left unimmunized and uninfected (naive group, n=10), immunized three times intradermally at monthly intervals with 20 μg poly C RNA-LNP (n=10) or with 20 μg each of an exemplary gC2, an exemplary gD2, an exemplary gE modified RNA-LNP (n=10). One month after the final immunization, animals in the poly C and trivalent RNA groups were infected intravaginally with 5×10⁵ PFU of HSV-2 strain MS (50 LD₅₀). Animals were observed for death, genital lesions during the acute phase of infection (days 1-14) and genital lesions during the recurrent phase of infection (days 15-60). FIG. 15A presents data on survival; FIG. 15B provides data on vaginal disease (acute phase); and FIG. 15C provides data on vaginal disease (recurrent phase).

FIGS. 16A-16D. Exemplary tri-HSV-1 and exemplary tri-HSV-2 immunizations generate antibodies that bind to HSV-1 proteins and neutralize HSV-1. Mice were immunized twice with PBS, exemplary tri-HSV-1 RNA, or exemplary tri-HSV-2 RNA at 1 μg or 10 μg of total RNA. FIGS. 16A-16C. IgG ELISA endpoint titers against HSV-1 proteins. FIG. 16D. HSV-1 neutralizing titers in the presence of 5% human serum as source of complement. N=10 mice per group. P values were calculated using the two-tailed Mann-Whitney test.

FIG. 17. Gating strategy used in CD4 and CD8 T cell flow cytometry analysis. Singlet cells were gated, then live CD3+, then lymphocytes, and then CD4+ and CD8+ T-cells. Single cytokine positive CD8+ T-cells were gated using CD8+ and TNFα, IFNγ, or IL-2 parameters. Gate coordinates were set based on the DMSO treated cells and those gate coordinates were transferred to the peptide treated cells.

FIGS. 18A-18D. Exemplary tri-HSV-1 and exemplary tri-HSV-2 immunizations generate CD4+ and CD8+ T-cell responses to HSV-1 overlapping peptides. Mice were immunized twice with 10 μg total of exemplary tri-HSV-1 RNA, exemplary tri-HSV-2 RNA, or PBS. FIG. 18A. Proportion of CD8+ T-cells producing 0, 1, 2, or 3 cytokines. Numbers inside or adjacent to colored segments indicate percentage of total CD8+ T-cells producing the indicated number of cytokines. FIG. 18B. Activation markers of CD8+ T-cells stimulated with gE1 overlapping peptide pool producing TNFα, IFNγ, and/or IL-2. FIG. 18C. Proportion of CD8+ T-cells expressing CD107a when stimulated with gE1 overlapping peptides. FIG. 18D. Proportion of CD4+ T-cells producing polyfunctional cytokines TNFα, IFNγ, and IL-2 following stimulation with a gD1 overlapping peptide pool. N=5 mice per group. P values in FIG. 18B were calculated using 2-way ANOVA with Tukey's correction for multiple comparisons. P values in FIG. 18C and FIG. 18D were calculated using the two-tailed Mann-Whitney test.

FIGS. 19A-19G. Exemplary tri-HSV-1 and tri-HSV-2 RNA compositions protect mice against acute and latent HSV-1 infection in the lip. Mice were immunized with PBS, exemplary tri-HSV-1 RNA, or tri-HSV-2 RNA compositions as in FIG. 1. Mice were challenged with 2×10⁶ PFU of HSV-1 applied to the scarified lower lip. FIG. 19A. HSV-1 virus titers in the lower lip. FIG. 19B. HSV-1 DNA copy number in the lower lip. FIG. 19C. HSV-1 virus titers in the trigeminal ganglia (TG). FIG. 19D. HSV-1 DNA copy number in the TG. FIGS. 19A-19D. n=10 per group. FIG. 19E. Mice were weighed daily for 13 days. FIG. 19F. Mice were scored for lesions on the lip with a score of 1 for lesions or 0 for no lesions. FIG. 19G. HSV-1 DNA genome copies in the TG 28 days post-challenge. FIGS. 19E-19G. n=24 mice for PBS group, n=15 mice per RNA immunization group. P values in FIGS. 19A-19D, and FIG. 19G were calculated by the two-tailed Mann-Whitney test.

FIGS. 20A-20F. Exemplary tri-HSV-1 and exemplary tri-HSV-2 RNA compositions protect mice from intravaginal HSV-1 disease. Mice were immunized with PBS, exemplary tri-HSV-1 RNA, or exemplary tri-HSV-2 RNA. Mice were challenged with 2×10⁶ PFU of HSV-1 intravaginally and scored for clinical disease for 14 days. FIG. 20A. Survival curve. P values were calculated by the log-rank test. FIG. 20B. Weight loss. FIG. 20C. Mean genital disease scores for each group. FIGS. 20D-20E. Vaginal swabs were obtained on days two and four for virus titers. FIG. 20F. HSV-1 DNA copy number in DRG. N=5 mice for PBS group, n=5 mice for tri-HSV-1 group, and n=4 mice for tri-HSV-2 group. P values in FIGS. 20D-20F were calculated by the two-tailed Mann-Whitney test without adjustments for multiple comparisons.

FIG. 21. Determination of LD₅₀ of HSV-1. Naïve (not vaccinated) 6 to 8-week-old female mice were inoculated intranasally with 5×10³, 5×10⁴, 5×10⁵, 5×10⁶ plaque-forming units (pfu) of HSV-1 strain H129, or with PBS as a control. Survival was evaluated over 28 days. Survival was 50% (2 of 4 mice) at 5×10³ pfu, establishing the 50% lethal dose (LD₅₀) as 5×10³ pfu.

FIGS. 22A-22D. Trivalent HSV-2 vaccine (Tri-HSV-2) protects BALB/c mice against HSV-1 encephalitis. Female BALB/c mice were immunized twice with either 1 g (0.33 g of each mRNA) or 10 g (3.33 g each mRNA) trivalent HSV-2 mRNA vaccine at 1-month intervals or PBS as described above. One month after the 2^{nd} immunization, animals were infected intranasally with 5×10⁵ pfu (100 LD₅₀) of HSV-1 strain H129. FIG. 22A. Animals were monitored for survival; P<0.01 comparing 1 or 10 g Tri-HSV-2 vaccine group with PBS by Log-rank test. FIGS. 22B-22D. HSV-1 DNA copy number was determined on day 6 post-infection by qPCR in brains (FIG. 22B), trigeminal ganglia (FIG. 22C), and olfactory bulbs (FIG. 22D); n=10/group except n=9 for olfactory bulb in the 1 g Tri-HSV-2 vaccine group. P value comparing log₁₀ copy number (within the graph): ***, P<0.001; P values were calculated by two-tailed Mann-Whitney test. P value comparing number of animals positive for HSV-1 DNA (above the graph): **, P<0.01; P values were calculated by two-tailed Fisher's exact test.

### DETAILED DESCRIPTION OF THE INVENTION

### Compositions

In one embodiment, the present invention provides compositions comprising one or more modified mRNAs, wherein each of said modified mRNAs encodes a Herpes Simplex Virus (HSV) glycoprotein or immunogenic fragment thereof.

In one embodiment, the present invention provides a composition comprising one or more nucleoside modified mRNAs, wherein each of said modified mRNAs encodes a Herpes Simplex Virus (HSV) glycoprotein or immunogenic fragment thereof, and wherein said modified mRNA comprises one or more pseudouridine or pseudouridine family residues.

In one embodiment, the HSV glycoprotein comprises glycoprotein D (gD), glycoprotein C (gC), glycoprotein E (gE), glycoprotein B (gB), glycoprotein H (gH), glycoprotein L (gL) glycoprotein I (gI), or a combination thereof.

Thus, in one embodiment, the present invention provides a composition comprising one or more modified mRNAs encoding HSV gD, gC, gE, gB, gH, gL, gI, or immunogenic fragments thereof. In one embodiment, the modified mRNAs comprise pseudouridine-modified mRNAs.

In one embodiment, the present invention provides compositions comprising a modified mRNA encoding HSV gD or fragment thereof. In another embodiment, the present invention provides compositions comprising a modified mRNA encoding HSV gC or fragment thereof. In another embodiment, the present invention provides compositions comprising a modified mRNA encoding HSV gE or fragment thereof. In another embodiment, the present invention provides compositions comprising a modified mRNA encoding HSV gB or fragment thereof. In another embodiment, the present invention provides compositions comprising a modified mRNA encoding HSV gH or fragment thereof. In another embodiment, the present invention provides compositions comprising a modified mRNA encoding HSV gL or fragment thereof. In another embodiment, the present invention provides compositions comprising a modified mRNA encoding HSV gI or fragment thereof.

In one embodiment, the present invention provides a composition comprising: (a) a modified mRNA encoding HSV gD or fragment thereof, and (b) a modified mRNA encoding HSV gC or fragment thereof.

In another embodiment, the present invention provides a composition comprising: (a) a modified mRNA encoding HSV gD or fragment thereof, and (b) a modified mRNA encoding HSV gE or fragment thereof.

In another embodiment, the present invention provides a composition comprising: (a) a modified mRNA encoding HSV gC or fragment thereof, and (b) a modified mRNA encoding HSV gE or fragment thereof.

In another embodiment, the present invention provides a composition comprising: (a) a modified mRNA encoding HSV gD or fragment thereof, (b) a modified mRNA encoding HSV gC or fragment thereof, and (c) a modified mRNA encoding HSV gE or fragment thereof.

In another embodiment, the present invention provides a composition comprising: (a) a modified mRNA encoding HSV gD or fragment thereof, (b) a modified mRNA encoding HSV gC or fragment thereof, (c) a modified mRNA encoding HSV gE or fragment thereof, and (d) a modified mRNA encoding HSV gB or fragment thereof.

In one embodiment, the HSV glycoproteins are HSV-2 glycoproteins. In another embodiment, the HSV glycoproteins are HSV-1 glycoproteins. In one embodiment, the HSV glycoproteins comprise both HSV-2 glycoproteins and HSV-1 glycoproteins. In another embodiment, the HSV glycoproteins comprise a mixture of HSV-2 glycoproteins and HSV-1 glycoproteins.

In one embodiment, the present invention provides compositions comprising a modified mRNA encoding HSV-2 gD or fragment thereof. In another embodiment, the present invention provides compositions comprising a modified mRNA encoding HSV-2 gC or fragment thereof. In another embodiment, the present invention provides compositions comprising a modified mRNA encoding HSV-2 gE or fragment thereof. In another embodiment, the present invention provides compositions comprising a modified mRNA encoding HSV-2 gE or fragment thereof. In another embodiment, the present invention provides compositions comprising a modified mRNA encoding HSV-2 gB or fragment thereof. In another embodiment, the present invention provides compositions comprising a modified mRNA encoding HSV-2 gH or fragment thereof. In another embodiment, the present invention provides compositions comprising a modified mRNA encoding HSV-2 gL or fragment thereof. In another embodiment, the present invention provides compositions comprising a modified mRNA encoding HSV-2 gI or fragment thereof.

In one embodiment, the present invention provides a composition comprising: (a) a modified mRNA encoding HSV-2 gD or fragment thereof, and (b) a modified mRNA encoding HSV-2 gC or fragment thereof.

In another embodiment, the present invention provides a composition comprising: (a) a modified mRNA encoding HSV-2 gD or fragment thereof, and (b) a modified mRNA encoding HSV-2 gE or fragment thereof.

In another embodiment, the present invention provides a composition comprising: (a) a modified mRNA encoding HSV-2 gC or fragment thereof, and (b) a modified mRNA encoding HSV-2 gE or fragment thereof.

In another embodiment, the present invention provides a composition comprising: (a) a modified mRNA encoding HSV-2 gD or fragment thereof, (b) a modified mRNA encoding HSV-2 gC or fragment thereof, and (c) a modified mRNA encoding HSV-2 gE or fragment thereof.

In one embodiment, the present invention provides compositions comprising a modified mRNA encoding HSV-1 gD or fragment thereof. In another embodiment, the present invention provides compositions comprising a modified mRNA encoding HSV-1 gC or fragment thereof. In another embodiment, the present invention provides compositions comprising a modified mRNA encoding HSV-1 gE or fragment thereof. In another embodiment, the present invention provides compositions comprising a modified mRNA encoding HSV-1 gE or fragment thereof. In another embodiment, the present invention provides compositions comprising a modified mRNA encoding HSV-1 gB or fragment thereof. In another embodiment, the present invention provides compositions comprising a modified mRNA encoding HSV-1 gH or fragment thereof. In another embodiment, the present invention provides compositions comprising a modified mRNA encoding HSV-1 gL or fragment thereof. In another embodiment, the present invention provides compositions comprising a modified mRNA encoding HSV-1 gI or fragment thereof.

In one embodiment, the present invention provides a composition comprising: (a) a modified mRNA encoding HSV-1 gD or fragment thereof, and (b) a modified mRNA encoding HSV-1 gC or fragment thereof.

In another embodiment, the present invention provides a composition comprising: (a) a modified mRNA encoding HSV-1 gD or fragment thereof, and (b) a modified mRNA encoding HSV-1 gE or fragment thereof.

In another embodiment, the present invention provides a composition comprising: (a) a modified mRNA encoding HSV-1 gC or fragment thereof, and (b) a modified mRNA encoding HSV-1 gE or fragment thereof.

In another embodiment, the present invention provides a composition comprising: (a) a modified mRNA encoding HSV-1 gD or fragment thereof, (b) a modified mRNA encoding HSV-1 gC or fragment thereof, and (c) a modified mRNA encoding HSV-1 gE or fragment thereof.

In one embodiment, any of the compositions as described herein consists essentially of one or more modified mRNAs, wherein each of said modified mRNAs encodes an HSV glycoprotein or immunogenic fragment thereof. In another embodiment, any of the compositions as described herein consists of one or more modified mRNAs, wherein each of said modified mRNAs encodes an HSV glycoprotein or immunogenic fragment thereof.

In another embodiment, the present invention provides compositions comprising a modified mRNA encoding an HSV gD protein, a modified mRNA encoding an HSV gC protein, a modified mRNA encoding an HSV gE protein and modified mRNAs encoding one or more additional HSV glycoproteins. In one embodiment, said additional HSV glycoproteins comprise gB or immunogenic fragment thereof, gH or immunogenic fragment thereof, gL or immunogenic fragment thereof, gI or immunogenic fragment thereof, or any combination thereof. In one embodiment, said additional HSV glycoproteins comprise glycoprotein M (gM), glycoprotein N (gN), glycoprotein K (gK), glycoprotein G (gG), glycoprotein J (gJ), or an immunogenic fragment thereof.

In one embodiment, compositions of the present invention and for use in the methods of the present invention comprise both HSV-2 glycoproteins or glycoprotein fragments and HSV-1 glycoproteins or glycoprotein fragments. In another embodiment, compositions of the present invention and for use in the methods of the present invention comprise a mixture of HSV-2 glycoproteins or glycoprotein fragments and HSV-1 glycoproteins or glycoprotein fragments. For example, in one embodiment, a composition of the present invention comprises HSV-2 gC, HSV-1 gD, and HSV-2 gE, or fragments thereof. In another embodiment, a composition of the present invention comprises HSV-1 gC, HSV-2 gD, and HSV-2 gE, or fragments thereof. In another embodiment, a composition of the present invention comprises HSV-2 gC, HSV-2 gD, and HSV-1 gE, or fragments thereof. In another embodiment, a composition of the present invention comprises HSV-1 gC, HSV-1 gD, and HSV-2 gE, or fragments thereof. In another embodiment, a composition of the present invention comprises HSV-1 gC, HSV-2 gD, and HSV-1 gE, or fragments thereof. In another embodiment, a composition of the present invention comprises HSV-2 gC, HSV-1 gD, and HSV-1 gE, or fragments thereof.

In another embodiment, the compositions of the present invention comprise one or more additional HSV-1 glycoproteins or HSV-2 glycoproteins or both HSV-1 and HSV-2 glycoproteins, as described herein. For example, in one embodiment, a composition of the present invention comprising HSV-2 gC, HSV-1 gD, and HSV-2 gE may further comprise HSV-1 gI. In another embodiment, a composition of the present invention comprising HSV-2 gC, HSV-2 gD, and HSV-2 gE may further comprise HSV-1 gB. Each of the possible combinations of HSV-1 and HSV-2 glycoproteins represents a separate embodiment of the invention.

“Encoding” refers, in one embodiment, to an RNA molecule that contains a gene that encodes the protein of interest. In another embodiment, the RNA molecule comprises a protein coding sequence that encodes the protein of interest. In another embodiment, one or more other proteins is also encoded. In another embodiment, the protein of interest is the only protein encoded. Each possibility represents a separate embodiment of the present invention.

“Immunogenic fragment” refers, in another embodiment, to a portion of a protein that is immunogenic and elicits a protective immune response when administered to a subject.

In one embodiment, “immunogenicity” or “immunogenic” is used herein to refer to the innate ability of a protein, peptide, nucleic acid, antigen or organism to elicit an immune response in an animal when the protein, peptide, nucleic acid, antigen or organism is administered to the animal. Thus, “enhancing the immunogenicity” in one embodiment, refers to increasing the ability of a protein, peptide, nucleic acid, antigen or organism to elicit an immune response in an animal when the protein, peptide, nucleic acid, antigen or organism is administered to an animal. The increased ability of a protein, peptide, nucleic acid, antigen or organism to elicit an immune response can be measured by, in one embodiment, a greater number of antibodies to a protein, peptide, nucleic acid, antigen or organism, a greater diversity of antibodies to an antigen or organism, a greater number of T-cells specific for a protein, peptide, nucleic acid, antigen or organism, a greater cytotoxic or helper T-cell response to a protein, peptide, nucleic acid, antigen or organism, and the like.

In one embodiment, an immunogenic polypeptide is also antigenic. “Antigenic” refers, in another embodiment, to a peptide capable of specifically interacting with an antigen recognition molecule of the immune system, e.g. an immunoglobulin (antibody) or T cell antigen receptor. An antigenic peptide contains, in another embodiment, an epitope of at least about 8 amino acids (AA). An antigenic portion of a polypeptide, also called herein the epitope in one embodiment, can be that portion that is immunodominant for antibody or T cell receptor recognition, or it can be a portion used to generate an antibody to the molecule by conjugating the antigenic portion to a carrier polypeptide for immunization. A molecule that is antigenic need not be itself immunogenic, i.e., capable of eliciting an immune response without a carrier.

In one embodiment, “functional” within the meaning of the invention, is used herein to refer to the innate ability of a protein, peptide, nucleic acid, fragment or a variant thereof to exhibit a biological activity or function. In one embodiment, such a biological function is its binding property to an interaction partner, e.g., a membrane-associated receptor, and in another embodiment, its trimerization property. In the case of functional fragments and the functional variants of the invention, these biological functions may in fact be changed, e.g., with respect to their specificity or selectivity, but with retention of the basic biological function.

In one embodiment, the term “fragment” is used herein to refer to a protein or polypeptide that is shorter or comprises fewer amino acids than the full length protein or polypeptide. In another embodiment, fragment refers to a nucleic acid encoding the protein fragment that is shorter or comprises fewer nucleotides than the full length nucleic acid. In another embodiment, the fragment is an N-terminal fragment. In another embodiment, the fragment is a C-terminal fragment. In one embodiment, the fragment is an intrasequential section of the protein, peptide, or nucleic acid. In another embodiment, the fragment is an immunogenic intrasequential section of the protein, peptide or nucleic acid. In another embodiment, the fragment is a functional intrasequential section within the protein, peptide or nucleic acid. In another embodiment, the fragment is an N-terminal immunogenic fragment. In one embodiment, the fragment is a C-terminal immunogenic fragment. In another embodiment, the fragment is an N-terminal functional fragment. In another embodiment, the fragment is a C-terminal functional fragment. In another embodiment, the fragment contains pieces of the protein linked together or pieces of multiple proteins linked together.

Thus, in one embodiment, an “immunogenic fragment” of a protein as described in the present invention refers to a portion of the protein that is immunogenic, in one embodiment and in another embodiment, elicits a protective immune response when administered to a subject.

In another aspect, the present invention provides compositions comprising modified mRNAs, wherein each of said modified mRNAs encodes a) HSV glycoprotein D (gD) or an immunogenic fragment thereof, b) HSV glycoprotein C (gC) or an immunogenic fragment thereof, c) HSV glycoprotein E (gE) or an immunogenic fragment thereof, or any combination thereof.

In one embodiment, the present invention provides a composition comprising a modified mRNA encoding an HSV gD or an immunogenic fragment thereof, a modified mRNA encoding an HSV gC or an immunogenic fragment thereof, and a modified mRNA encoding an HSV gE or an immunogenic fragment thereof.

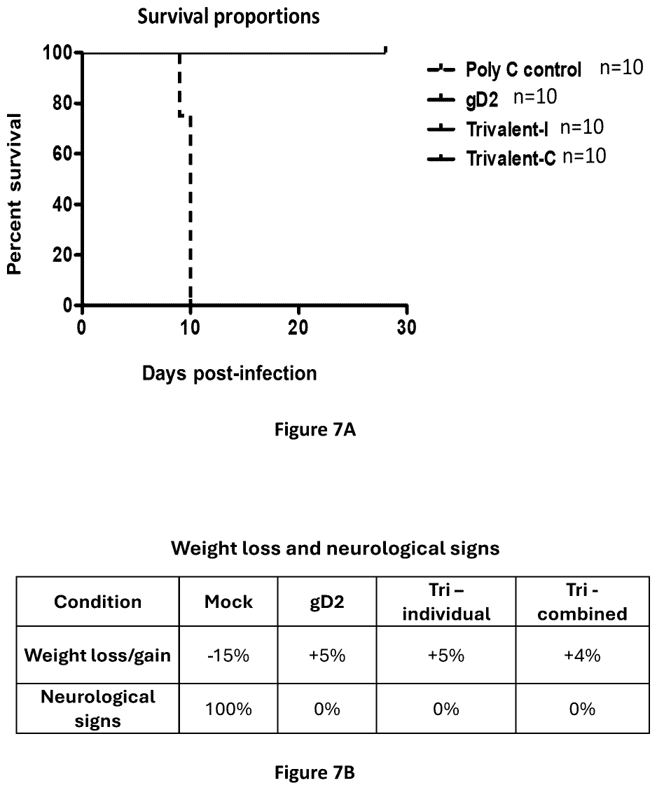

In one embodiment, compositions of modified mRNA encoding gD-1 are protective against HSV-1 infection. Further, combination compositions of modified mRNA encoding gC-1/gD-1/gE-1 confer superior protection compared with compositions containing modified mRNA encoding gC-1 alone, gD-1 alone, or gE-1 alone. Further, as provided herein, compositions of modified mRNA encoding gD-2 are protective against HSV-2 infection (FIGS. 7-10). Further, combination compositions of modified mRNA encoding gC-2/gD-2/gE-2 confer superior protection compared with compositions containing modified mRNA encoding gC-2 alone, gD-2 alone, or gE-2 alone.

In another embodiment, inclusion of a modified mRNA encoding gC, and/or a modified mRNA encoding gE in the composition of the present invention increases the efficaciousness of anti-gD antibodies elicited by the composition. In another embodiment, inclusion of a modified mRNA encoding gC, and/or a modified mRNA encoding gE in the composition of the present invention increases the dose of modified mRNA encoding gD required to elicit antibodies that inhibit binding of gD to a cellular receptor. In another embodiment, inclusion of a modified mRNA encoding gC, and/or a modified mRNA encoding gE in the composition of the present invention decreases the dose of modified mRNA encoding gD required to elicit antibodies that inhibit binding of gD to a cellular receptor when a dose of modified mRNA encoding a gD is administered separately from modified mRNAs encoding the gC protein or gE protein.

In another embodiment, inclusion of a modified mRNA encoding gC, and/or a modified mRNA encoding gE in the composition of the present invention enhances the effectiveness of an innate immune response. In another embodiment, the innate immune response is an antibody-mediated immune response. In another embodiment, the innate immune response is a non-antibody-mediated immune response. In another embodiment, the innate immune response is an NK (natural killer) cell response. In another embodiment, the innate immune response is any other innate immune response known in the art.

In another embodiment, inclusion of a modified mRNA encoding gC, and/or a modified mRNA encoding gE in the composition of the present invention increases the efficaciousness of antibodies elicited by the composition against one of the above glycoproteins. In another embodiment, inclusion of a modified mRNA encoding gC, and/or a modified mRNA encoding gE in the composition of the present invention decreases the dose of one of the above glycoproteins required to elicit antibodies that inhibit binding of the glycoprotein to a cellular receptor thereof, when a dose of one of the glycoproteins is administered separately from one of the other glycoproteins.

### Glycoprotein D

In one embodiment, a composition of the present invention comprises a modified mRNA encoding HSV-1 gD protein. In another embodiment, the composition comprises a modified mRNA encoding a fragment of an HSV-1 gD protein.

In one embodiment, the nucleotide sequence of the modified mRNA encoding an HSV-1 gD fragment comprises:

(SEQ ID NO: 1)
GGAAUAAAAGUCUCAACACAACAUAUACAAAACAAACGAAUCUCAAGCA
AUCAAGCAUUCUACUUCUAUUGCAGCAAUUUAAAUCAUUUCUUUUAAAG
CAAAAGCAAUUUUCUGAAAAUUUUCACCAUUUACGAACGAUAGCAUGCG
CAUGCAGCUGCUGCUGCUGAUCGCCCUGUCCCUGGCCCUGGUGACCAAC
UCCAAGUACGCCCUGGCCGACGCCUCCCUGAAGAUGGCCGACCCCAACC
GCUUCCGCGGCAAGGACCUGCCCGUGCUGGACCAGCUGACCGACCCCCC
CGGCGUGCGCCGCGUGUACCACAUCCAGGCCGGCCUGCCCGACCCCUUC
CAGCCCCCCUCCCUGCCCAUCACCGUGUACUACGCCGUGCUGGAGCGCG
CCUGCCGCUCCGUGCUGCUGAACGCCCCCUCCGAGGCCCCCCAGAUCGU
GCGCGGCGCCUCCGAGGACGUGCGCAAGCAGCCCUACAACCUGACCAUC
GCCUGGUUCCGCAUGGGCGGCAACUGCGCCAUCCCCAUCACCGUGAUGG
AGUACACCGAGUGCUCCUACAACAAGUCCCUGGGCGCCUGCCCCAUCCG
CACCCAGCCCCGCUGGAACUACUACGACUCCUUCUCCGCCGUGUCCGAG
GACAACCUGGGCUUCCUGAUGCACGCCCCCGCCUUCGAGACCGCCGGCA
CCUACCUGCGCCUGGUGAAGAUCAACGACUGGACCGAGAUCACCCAGUU
CAUCCUGGAGCACCGCGCCAAGGGCUCCUGCAAGUACGCCCUGCCCCUG
CGCAUCCCCCCCUCCGCCUGCCUGUCCCCCCAGGCCUACCAGCAGGGCG
UGACCGUGGACUCCAUCGGCAUGCUGCCCCGCUUCAUCCCCGAGAACCA
GCGCACCGUGGCCGUGUACUCCCUGAAGAUCGCCGGCUGGCACGGCCCC
AAGGCCCCCUACACCUCCACCCUGCUGCCCCCCGAGCUGUCCGAGACCC
CCAACGCCACCCAGCCCGAGCUGGCCCCCGAGGACCCCGAGGACUCCGC
CCUGCUGGAGGACCCCGUGGGCACCGUGGCCCCCCAGAUCCCCCCCAAC
UGGCACAUCCCCUCCAUCCAGGACGCCGCCACCCCCUACUAACUAGUAG
UGACUGACUAGGAUCUGGUUACCACUAAACCAGCCUCAAGAACACCCGA
AUGGAGUCUCUAAGCUACAUAAUACCAACUUACACUUACAAAAUGUUGU
CCCCCAAAAUGUAGCCAUUCGUAUCUGCUCCUAAUAAAAAGAAAGUUUC
UUCACAUUCUAAAAAAAAAAAAAAAAAAAAAAAAAAAAAAAAAAAAAAA
AAAAAAAAAAAAAAAAAAAAAAAAAAAAAAAAAAAAAAAAAAAAAAAAA
AAAAAAAAAAAAAC

In one embodiment, all uridine residues are 1-methyl-pseudouridine. In one embodiment, underlined residues represent 5′ untranslated sequences. In one embodiment, bold residues represent a signal sequence (leader sequence) to assist expression of the gD1 fragment. In one embodiment, italicized residues represent 3′ untranslated sequences and poly adenylation tail.

In another embodiment, the nucleotide sequence of the modified mRNA encoding an HSV-1 gD fragment lacks the 5′ untranslated sequences, the signal sequence, the 3′ untranslated sequences, the poly adenylation tail, or a combination thereof.

In one embodiment, the HSV-1 gD fragment encoded by modified mRNA utilized in the methods and compositions of the present invention comprises amino acids 26-331 of gD from HSV-1 Patton strain, as set forth in the following amino acid sequence:

(SEQ ID NO: 2)
KYALADASLKMADPNRFRGKDLPVLDQLTDPPGVRRVYHIQAGLPDPFQ
PPSLPITVYYAVLERACRSVLLNAPSEAPQIVRGASEDVRKQPYNLTIA
WFRMGGNCAIPITVMEYTECSYNKSLGACPIRTQPRWNYYDSFSAVSED
NLGFLMHAPAFETAGTYLRLVKINDWTEITQFILEHRAKGSCKYALPLR
IPPSACLSPQAYQQGVTVDSIGMLPRFIPENQRTVAVYSLKIAGWHGPK
APYTSTLLPPELSETPNATQPELAPEDPEDSALLEDPVGTVAPQIPPNW
HIPSIQDAATPY

In one embodiment, the full length HSV-1 gD encoded by modified mRNA utilized in the methods and compositions of the present invention comprises the following amino acid sequence:

(SEQ ID NO: 3)
MGGAAARLGAVILFVVIVGLHGVRGKYALADASLKLADPNRFRRKDLPV
LDQLTDPPGVRRVYHIQAGLPDPFQPPSLPITVYYAVLERACRSVLLNA
PSEAPQIVRGASEDVRKQPYNLTIAWFRMGGNCAIPITVMEYTECSYNK
SLGACPIRTQPRWNYYDSFSAVSEDNLGFLMHAPAFETAGTYLRLVKIN
DWTEITQFILEHRAKGSCKYALPLRIPPSACLSPQAYQQGVTVDSIGML
PRFIPENQRTVAVYSLKIAGWHGPKAPYTSTLLPPELSETPNATQPELA
PEAPEDSALLEDPVGTVAPQIPPNWHIPSIQDAATPYHPPATPNNMGLI
AGAVGGSLLAALVICGIVYWMRRRTQKAPKRIRLPHIREDDQPSSHQPL
FY

In another embodiment, the HSV-1 gD encoded by modified mRNA utilized in the methods and compositions of the present invention comprises the amino acid sequences as set forth in any one of the following GenBank Accession Numbers: AAL90884.1 (KHS2 strain), AAL90883.1 (KHS1 strain), AAK93950.1 (F strain), AAB59754.1 (F strain), AAA19631.1 (mutant strain not identified), AAA19630.1 (mutant strain not identified), or AAA19629.1 (strain not identified).

In another embodiment, the HSV-1 gD encoded by modified mRNA utilized in the methods and compositions of the present invention comprises the amino acid sequences as set forth in any of the following GenBank Accession Numbers: A1Z0Q5.2, AAA45780.1, AAA45785.1, AAA45786.1, AAA96682.1, AAK19597.1, AAN74642.1, ABI63524.1, ABM52978.1, ABM52979.1, ABM52980.1, ABM52981.1, ABM66847.1, ABM66848.1, ACM62295.1, ADD60053.1, ADD60130.1, ADM22389.1, ADM22466.1, ADM22542.1, ADM22619.1, ADM22696.1, ADM22773.1, ADM22849.1, ADM22926.1, ADM23003.1, ADM23079.1, ADM23155.1, ADM23231.1, ADM23309.1, ADM23383.1, ADM23457.1, ADM23531.1, ADM23605.1, ADM23680.1, ADM23755.1, ADM23831.1, AEQ77097.1, AER37647.1, AER37715.1, AER37786.1, AER37857.1, AER37929.1, AER38000.1, AER38070.1, AFE62894.1, AFH41180.1, AFI23657.1, AFK50415.1, AFP86430.1, AGZ01928.1, AIR95858.1, AJE60009.1, AJE60080.1, AJE60151.1, AJE60222.1, AJE60293.1, AJE60439.1, AKE48645.1, AKG59246.1, AKG59318.1, AKG59391.1, AKG59462.1, AKG59536.1, AKG59609.1, AKG59682.1, AKG59755.1, AKG59826.1, AKG59898.1, AKG59972.1, AKG60046.1, AKG60118.1, AKG60189.1, AKG60261.1, AKG60334.1, AKG60404.1, AKG60474.1, AKG60546.1, AKG60620.1, AKG60692.1, AKG60763.1, AKG60835.1, AKG60906.1, AKG60978.1, AKG61050.1, AKG61123.1, AKG61194.1, AKG61267.1, AKG61339.1, AKG61411.1, AKG61484.1, AKG61556.1, AKG61629.1, AKG61703.1, AKG61774.1, AKG61847.1, AKG61920.1, AKG61993.1, AKH80463.1, AKH80536.1, ALM22635.1, ALM22709.1, ALM22783.1, ALM22857.1, ALO18662.1, ALO18738.1, AMB65662.1, AMB65735.1, AMB65809.1, AMB65885.1, AMB65956.1, AMN09832.1, ANN83964.1, ANN84041.1, ANN84117.1, ANN84194.1, ANN84271.1, ANN84348.1, ANN84424.1, ANN84500.1, ANN84577.1, ANN84653.1, ANN84730.1, ANN84806.1, ANN84883.1, ANN84959.1, ANN85036.1, ANN85112.1, ANN85187.1, ANN85264.1, ANN85341.1, ANN85416.1, ANN85494.1, ANN85571.1, ANN85648.1, ANN85724.1, ANN85801.1, AOY34093.1, AOY34141.1, AOY34243.1, AOY34271.1, AOY34337.1, AOY36685.1, ARB08957.1, ARO37961.1, ARO37962.1, ARO37963.1, ARO37964.1, ARO37965.1, ARO37966.1, ARO37967.1, ARO37968.1, ARO37969.1, ARO37970.1, ARO37971.1, ARO37972.1, ARO37973.1, ARO37974.1, ARO37975.1, ARO37976.1, ARO37977.1, ARO37978.1, ARO37979.1, ARO37980.1, ARO37981.1, ARO37982.1, ARO37983.1, ARO37984.1, ARO37985.1, ARO37986.1, ARO37987.1, ARO37988.1, ARO37989.1, ARO37990.1, ARO37991.1, ARO37992.1, ARO37993.1, ARO37994.1, ARO37995.1, ARO37996.1, ARO37997.1, ARO37998.1, ARO37999.1, ASM47664.1, ASM47741.1, ASM47818.1, ASM47893.1, BAM73419.1, CAA26060.1, CAA32283.1, CAA32284.1, CAA32289.1, CAA38245.1, CAT05431.1, P06476.1, P36318.1, P57083.1, P68331.1, Q05059.1, Q69091.1, SBO07792.1, SBO07819.1, SBO07855.1, SBO07869.1, SBO07887.1, SBO07908.1, SBS69553.1, SBS69561.1, SBS69579.1, SBS69625.1, SBS69688.1, SBS69694.1, SBS69717.1, SBS69727.1, SBS69811.1, SBT69395.1, SCL76902.1, VGBEDZ, or YP_009137141.1.

In another embodiment, the composition comprises a modified mRNA encoding an HSV-2 gD protein. In another embodiment, the composition comprises a modified mRNA encoding a fragment of an HSV-2 gD protein.

In one embodiment, the nucleotide sequence of the modified mRNA encoding an HSV-2 gD fragment comprises:

(SEQ ID NO: 4)
GGAAUAAAAGUCUCAACACAACAUAUACAAAACAAACGAAUCUCAAGCA
AUCAAGCAUUCUACUUCUAUUGCAGCAAUUUAAAUCAUUUCUUUUAAAG
CAAAAGCAAUUUUCUGAAAAUUUUCACCAUUUACGAACGAUAGCAUGAC
CCGCCUGACCGUGCUGGCCCUGCUGGCCGGCCUGCUGGCCUCCUCCCGC
GCCAAGUACGCCCUGGCCGACCCCUCCCUGAAGAUGGCCGACCCCAACC
GCUUCCGCGGCAAGAACCUGCCCGUGCUGGACCAGCUGACCGACCCCCC
CGGCGUGAAGCGCGUGUACCACAUCCAGCCCUCCCUGGAGGACCCCUUC
CAGCCCCCCUCCAUCCCCAUCACCGUGUACUACGCCGUGCUGGAGCGCG
CCUGCCGCUCCGUGCUGCUGCACGCCCCCUCCGAGGCCCCCCAGAUCGU
GCGCGGCGCCUCCGACGAGGCCCGCAAGCACACCUACAACCUGACCAUC
GCCUGGUACCGCAUGGGCGACAACUGCGCCAUCCCCAUCACCGUGAUGG
AGUACACCGAGUGCCCCUACAACAAGUCCCUGGGCGUGUGCCCCAUCCG
CACCCAGCCCCGCUGGUCCUACUACGACUCCUUCUCCGCCGUGUCCGAG
GACAACCUGGGCUUCCUGAUGCACGCCCCCGCCUUCGAGACCGCCGGCA
CCUACCUGCGCCUGGUGAAGAUCAACGACUGGACCGAGAUCACCCAGUU
CAUCCUGGAGCACCGCGCCCGCGCCUCCUGCAAGUACGCCCUGCCCCUG
CGCAUCCCCCCCGCCGCCUGCCUGACCUCCAAGGCCUACCAGCAGGGCG
UGACCGUGGACUCCAUCGGCAUGCUGCCCCGCUUCAUCCCCGAGAACCA
GCGCACCGUGGCCCUGUACUCCCUGAAGAUCGCCGGCUGGCACGGCCCC
AAGCCCCCCUACACCUCCACCCUGCUGCCCCCCGAGCUGUCCGACACCA
CCAACGCCACCCAGCCCGAGCUGGUGCCCGAGGACCCCGAGGACUCCGC
CCUGCUGGAGGACCCCGCCGGCACCGUGUCCUCCCAGAUCCCCCCCAAC
UGGCACAUCCCCUCCAUCCAGGACGUGGCCCCCCACCACUAACUAGUAG
UGACUGACUAGGAUCUGGUUACCACUAAACCAGCCUCAAGAACACCCGA
AUGGAGUCUCUAAGCUACAUAAUACCAACUUACACUUACAAAAUGUUGU
CCCCCAAAAUGUAGCCAUUCGUAUCUGCUCCUAAUAAAAAGAAAGUUUC
UUCACAUUCUAAAAAAAAAAAAAAAAAAAAAAAAAAAAAAAAAAAAAAA
AAAAAAAAAAAAAAAAAAAAAAAAAAAAAAAAAAAAAAAAAAAAAAAAA
AAAAAAAAAAAAAC

In one embodiment, all uridine residues are 1-methyl-pseudouridine. In one embodiment, underlined residues represent 5′ untranslated sequences. In one embodiment, bold residues represent a signal sequence (leader sequence) to assist expression of the gD2 fragment. In one embodiment, italicized residues represent 3′ untranslated sequences and poly adenylation tail.

In another embodiment, the nucleotide sequence of the modified mRNA encoding an HSV-2 gD fragment lacks the 5′ untranslated sequences, the signal sequence, the 3′ untranslated sequences, the poly adenylation tail, or a combination thereof.

In one embodiment, the HSV-2 gD fragment encoded by modified mRNA utilized in the methods and compositions of the present invention comprises amino acids 26-331 of gD from HSV-2 strain 333, as set forth in the following amino acid sequence:

(SEQ ID NO: 5)
KYALADPSLKMADPNRFRGKNLPVLDQLTDPPGVKRVYHIQPSLEDPFQ
PPSIPITVYYAVLERACRSVLLHAPSEAPQIVRGASDEARKHTYNLTIA
WYRMGDNCAIPITVMEYTECPYNKSLGVCPIRTQPRWSYYDSFSAVSED
NLGFLMHAPAFETAGTYLRLVKINDWTEITQFILEHRARASCKYALPLR
IPPAACLTSKAYQQGVTVDSIGMLPRFIPENQRTVALYSLKIAGWHGPK
PPYTSTLLPPELSDTTNATQPELVPEDPEDSALLEDPAGTVSSQIPPNW
HIPSIQDVAPHH.

In one embodiment, the full length HSV-2 gD encoded by modified mRNA utilized in the methods and compositions of the present invention comprises the following amino acid sequence:

(SEQ ID NO: 6)
MGRLTSGVGTAALLVVAVGLRVVCAKYALADPSLKMADPNRFRGKNLPV
LDQLTDPPGVKRVYHIQPSLEDPFQPPSIPITVYYAVLERACRSVLLHA
PSEAPQIVRGASDEARKHTYNLTIAWYRMGDNCAIPITVMEYTECPYNK
SLGVCPIRTQPRWSYYDSFSAVSEDNLGFLMHAPAFETAGTYLRLVKIN
DWTEITQFILEHRARASCKYALPLRIPPAACLTSKAYQQGVTVDSIGML
PRFIPENQRTVALYSLKIAGWHGPKPPYTSTLLPPELSDTTNATQPELV
PEDPEDSALLEDPAGTVSSQIPPNWHIPSIQDVAPHHAPAAPSNPGLII
GALAGSTLAVLVIGGIAFWVRRRAQMAPKRLRLPHIRDDDAPPSHQPLF
Y.

In another embodiment, the HSV-2 gD encoded by modified mRNA utilized in the methods and compositions of the present invention comprises the amino acid sequences as set forth in GenBank Accession Numbers: 1003204A, AAA45841.1, AAA45842.1, AAB60552.1, AAB60553.1, AAB60554.1, AAB60555.1, AAB72102.1, AAS01730.1, AAW23130.1, AAW23131.1, AAW23132.1, AAW23133.1, AAW23134.1, ABS84899.1, ABU45433.1, ABU45434.1, ABU45435.1, ABU45461.1, ABU45462.1, ACA28831.1, AEV91405.1, AFM93876.1, AFS18198.1, AFS18199.1, AFS18200.1, AFS18201.1, AFS18202.1, AFS18203.1, AFS18204.1, AFS18205.1, AFS18206.1, AFS18207.1, AFS18208.1, AFS18209.1, AFS18210.1, AFS18211.1, AFS18212.1, AFS18213.1, AFS18214.1, AFS18215.1, AFS18216.1, AFS18217.1, AFS18218.1, AFS18219.1, AFS18220.1, AFS18221.1, AHG54730.1, AIL27720.1, AIL27721.1, AIL27722.1, AIL27723.1, AIL27724.1, AIL27725.1, AIL27726.1, AIL27727.1, AIL27728.1, AIL27729.1, AIL27730.1, AIL27731.1, AIL28069.1, AIL28070.1, AKC42828.1, AKC59305.1, AKC59376.1, AKC59447.1, AKC59518.1, AKC59589.1, AMB66102.1, AMB66171.1, AMB66244.1, AMB66321.1, AMB66394.1, AMB66463.1, AQZ55754.1, AQZ55825.1, AQZ55896.1, AQZ55967.1, AQZ56038.1, AQZ56109.1, AQZ56180.1, AQZ56251.1, AQZ56322.1, AQZ56393.1, AQZ56464.1, AQZ56535.1, AQZ56606.1, AQZ56677.1, AQZ56748.1, AQZ56819.1, AQZ56890.1, AQZ56961.1, AQZ57032.1, AQZ57103.1, AQZ57174.1, AQZ57245.1, AQZ57316.1, AQZ57387.1, AQZ57458.1, AQZ57529.1, AQZ57600.1, AQZ57671.1, AQZ57742.1, AQZ57813.1, AQZ57884.1, AQZ57955.1, AQZ58026.1, AQZ58097.1, AQZ58168.1, AQZ58239.1, AQZ58310.1, AQZ58381.1, AQZ58452.1, AQZ58523.1, AQZ58594.1, AQZ58665.1, AQZ58736.1, AQZ58807.1, AQZ58878.1, AQZ58949.1, AQZ59020.1, AQZ59091.1, AQZ59162.1, ARO38000.1, ARO38001.1, ARO38002.1, ARO38003.1, ARO38004.1, ARO38005.1, ARO38006.1, ARO38007.1, ARO38008.1, ARO38009.1, ARO38010.1, ARO38011.1, ARO38012.1, ARO38013.1, ARO38014.1, ARO38015.1, ARO38016.1, ARO38017.1, ARO38018.1, ARO38019.1, ARO38020.1, ARO38021.1, ARO38022.1, ARO38023.1, ARO38024.1, ARO38025.1, ARO38026.1, ARO38027.1, ARO38028.1, ARO38029.1, ARO38030.1, ARO38031.1, ARO38032.1, ARO38033.1, ARO38034.1, ARO38035.1, ARO38036.1, ARO38037.1, ARO38038.1, ARO38039.1, ARO38040.1, ARO38041.1, ARO38042.1, ARO38043.1, ARO38044.1, CAA26025.1, CAB06713.1, CAC33573.1, CAT05432.1, P03172.2, Q69467.1, or YP_009137218.1.

In another embodiment, the gD protein or fragment includes Y63. In another embodiment, the gD protein or fragment includes R159. In another embodiment, the gD protein or fragment includes D240. In another embodiment, the gD protein or fragment includes P246. In another embodiment, the gD protein or fragment includes a residue selected from Y63, R159, D240, and P246. In another embodiment, inclusion of one of these residues elicits antibodies that inhibit binding to nectin-1.

The nomenclature used herein for gD amino acid residues includes the residues of the signal sequence. Thus, residue one of the mature protein is referred to as “26.”

Each modified mRNA encoding gD-1 and gD-2 protein or fragment thereof represents a separate embodiment of the present invention.

In another embodiment, the HSV gD, gC, and gE proteins, and fragments thereof, encoded by the modified mRNA as disclosed herein are described in US Patent Publication No. 2013-0028925-A1, which is incorporated by reference herein in its entirety.

In another embodiment, a gD protein fragment encoded by modified mRNA utilized in the methods and compositions of the present invention is an immunogenic fragment. In another embodiment, a gD immunoprotective antigen need not be the entire protein. The protective immune response generally involves, in another embodiment, an antibody response. In another embodiment, mutants, sequence conservative variants, and functional conservative variants of gD are useful in methods and compositions of the present invention, provided that all such variants retain the required immuno-protective effect. In another embodiment, the immunogenic fragment can comprise an immuno-protective gD antigen from any strain of HSV. In another embodiment, the immunogenic fragment can comprise sequence variants of HSV, as found in infected individuals.

### Glycoprotein C

In another embodiment, a composition of the present invention comprises a modified mRNA encoding HSV-1 gC protein. In another embodiment, the composition comprises a modified mRNA encoding a fragment of an HSV-1 gC protein.

In one embodiment, the nucleotide sequence of the modified mRNA encoding an HSV-1 gC fragment comprises:

(SEQ ID NO: 7)
GGAAUAAAAGUCUCAACACAACAUAUACAAAACAAACGAAUCUCAAGCA
AUCAAGCAUUCUACUUCUAUUGCAGCAAUUUAAAUCAUUUCUUUUAAAG
CAAAAGCAAUUUUCUGAAAAUUUUCACCAUUUACGAACGAUAGCAUGGC
CAUCUCCGGCGUGCCCGUGCUGGGCUUCUUCAUCAUCGCCGUGCUGAUG
UCCGCCCAGGAGUCCUGGGCCGAGACCGCCUCCACCGGCCCCACCAUCA
CCGCCGGCGCCGUGACCAACGCCUCCGAGGCCCCCACCUCCGGCUCCCC
CGGCUCCGCCGCCUCCCCCGAGGUGACCCCCACCUCCACCCCCAACCCC
AACAACGUGACCCAGAACAAGACCACCCCCACCGAGCCCGCCUCCCCCC
CCACCACCCCCAAGCCCACCUCCACCCCCAAGUCCCCCCCCACCUCCAC
CCCCGACCCCAAGCCCAAGAACAACACCACCCCCGCCAAGUCCGGCCGC
CCCACCAAGCCCCCCGGCCCCGUGUGGUGCGACCGCCGCGACCCCCUGG
CCCGCUACGGCUCCCGCGUGCAGAUCCGCUGCCGCUUCCGCAACUCCAC
CCGCAUGGAGUUCCGCCUGCAGAUCUGGCGCUACUCCAUGGGCCCCUCC
CCCCCCAUCGCCCCCGCCCCCGACCUGGAGGAGGUGCUGACCAACAUCA
CCGCCCCCCCCGGCGGCCUGCUGGUGUACGACUCCGCCCCCAACCUGAC
CGACCCCCACGUGCUGUGGGCCGAGGGCGCCGGCCCCGGCGCCGACCCC
CCCCUGUACUCCGUGACCGGCCCCCUGCCCACCCAGCGCCUGAUCAUCG
GCGAGGUGACCCCCGCCACCCAGGGCAUGUACUACCUGGCCUGGGGCCG
CAUGGACUCCCCCCACGAGUACGGCACCUGGGUGCGCGUGCGCAUGUUC
CGCCCCCCCUCCCUGACCCUGCAGCCCCACGCCGUGAUGGAGGGCCAGC
CCUUCAAGGCCACCUGCACCGCCGCCGCCUACUACCCCCGCAACCCCGU
GGAGUUCGACUGGUUCGAGGACGACCGCCAGGUGUUCAACCCCGGCCAG
AUCGACACCCAGACCCACGAGCACCCCGACGGCUUCACCACCGUGUCCA
CCGUGACCUCCGAGGCCGUGGGCGGCCAGGUGCCCCCCCGCACCUUCAC
CUGCCAGAUGACCUGGCACCGCGACUCCGUGACCUUCUCCCGCCGCAAC
GCCACCGGCCUGGCCCUGGUGCUGCCCCGCCCCACCAUCACCAUGGAGU
UCGGCGUGCGCCACGUGGUGUGCACCGCCGGCUGCGUGCCCGAGGGCGU
GACCUUCGCCUGGUUCCUGGGCGACGACCCCUCCCCCGCCGCCAAGUCC
GCCGUGACCGCCCAGGAGUCCUGCGACCACCCCGGCCUGGCCACCGUGC
GCUCCACCCUGCCCAUCUCCUACGACUACUCCGAGUACAUCUGCCGCCU
GACCGGCUACCCCGCCGGCAUCCCCGUGCUGGAGCACCACUAACUAGUA
GUGACUGACUAGGAUCUGGUUACCACUAAACCAGCCUCAAGAACACCCG
AAUGGAGUCUCUAAGCUACAUAAUACCAACUUACACUUACAAAAUGUUG
UCCCCCAAAAUGUAGCCAUUCGUAUCUGCUCCUAAUAAAAAGAAAGUUU
CUUCACAUUCUAAAAAAAAAAAAAAAAAAAAAAAAAAAAAAAAAAAAAA
AAAAAAAAAAAAAAAAAAAAAAAAAAAAAAAAAAAAAAAAAAAAAAAAA
AAAAAAAAAAAAAAC

In one embodiment, all uridine residues are 1-methyl-pseudouridine. In one embodiment, underlined residues represent 5′ untranslated sequences. In one embodiment, bold residues represent a signal sequence (leader sequence) to assist expression of the gC1 fragment. In one embodiment, italicized residues represent 3′ untranslated sequences and poly adenylation tail.

In another embodiment, the nucleotide sequence of the modified mRNA encoding an HSV-1 gC fragment does not comprise the 5′ untranslated sequences, the signal sequence, the 3′ untranslated sequences, the poly adenylation tail, or a combination thereof.

In one embodiment, the HSV-1 gC fragment encoded by modified mRNA utilized in the methods and compositions of the present invention comprises amino acids 27-457 of gC from HSV-1 KOS strain, as set forth in the following amino acid sequence:

(SEQ ID NO: 8)
ETASTGPTITAGAVTNASEAPTSGSPGSAASPEVTPTSTPNPNNVTQNK
TTPTEPASPPTTPKPTSTPKSPPTSTPDPKPKNNTTPAKSGRPTKPPGP
VWCDRRDPLARYGSRVQIRCRFRNSTRMEFRLQIWRYSMGPSPPIAPAP
DLEEVLTNITAPPGGLLVYDSAPNLTDPHVLWAEGAGPGADPPLYSVTG
PLPTQRLIIGEVTPATQGMYYLAWGRMDSPHEYGTWVRVRMFRPPSLTL
QPHAVMEGQPFKATCTAAAYYPRNPVEFDWFEDDRQVENPGQIDTQTHE
HPDGFTTVSTVTSEAVGGQVPPRTFTCQMTWHRDSVTFSRRNATGLALV
LPRPTITMEFGVRHVVCTAGCVPEGVTFAWFLGDDPSPAAKSAVTAQES
CDHPGLATVRSTLPISYDYSEYICRLTGYPAGIPVLEHH.

In one embodiment, the gC fragment encoded by modified mRNA utilized in the methods and compositions of the present invention comprises amino acids 27-457 of gC from an HSV-1 strain.

In one embodiment, the full length HSV-1 gC encoded by modified mRNA utilized in the methods and compositions of the present invention comprises the following amino acid sequence:

(SEQ ID NO: 9)
MAPGRVGLAVVLWGLLWLGAGVAGGSETASTGPTITAGAVTNASEAPTS
GSPGSAASPEVTPTSTPNPNNVTQNKTTPTEPASPPTTPKPTSTPKSPP
TSTPDPKPKNNTTPAKSGRPTKPPGPVWCDRRDPLARYGSRVQIRCRFR
NSTRMEFRLQIWRYSMGPSPPIAPAPDLEEVLTNITAPPGGLLVYDSAP
NLTDPHVLWAEGAGPGADPPLYSVTGPLPTQRLIIGEVTPATQGMYYLA
WGRMDSPHEYGTWVRVRMFRPPSLTLQPHAVMEGQPFKATCTAAAYYPR
NPVEFDWFEDDRQVFNPGQIDTQTHEHPDGFTTVSTVTSEAVGGQVPPR
TFTCQMTWHRDSVTFSRRNATGLALVLPRPTITMEFGVRHVVCTAGCVP
EGVTFAWFLGDDPSPAAKSAVTAQESCDHPGLATVRSTLPISYDYSEYI
CRLTGYPAGIPVLEHHGSHQPPPRDPTERQVIEAIEWVGIGIGVLAAGV
LVVTAIVYVVRTSQSRQRHRR.

In another embodiment, the HSV-1 gC encoded by modified mRNA utilized in the methods and compositions of the present invention comprises the amino acid sequences as set forth in any of the following GenBank Accession Numbers: AAA45779.1, AAA96680.1, ABI63505.1, ABM52973.1, ABM52976.1, ABM52977.1, ACM62267.1, ADD60042.1, ADD60119.1, ADM22367.1, ADM22444.1, ADM22520.1, ADM22597.1, ADM22674.1, ADM22751.1, ADM22827.1, ADM22904.1, ADM22981.1, ADM23057.1, ADM23133.1, ADM23210.1, ADM23287.1, ADM23361.1, ADM23435.1, ADM23509.1, ADM23583.1, ADM23658.1, ADM23733.1, ADM23809.1, AEQ77075.1, AEQ77099.1, AER37628.1, AER37697.1, AER37767.1, AER37838.1, AER37910.1, AER37981.1, AER38051.2, AFA36179.1, AFA36180.1, AFA36181.1, AFA36182.1, AFA36183.1, AFA36184.1, AFA36185.1, AFA36186.1, AFA36187.1, AFA36188.1, AFA36189.1, AFA36190.1, AFA36191.1, AFA36192.1, AFA36193.1, AFA36194.1, AFA36195.1, AFA36196.1, AFA36197.1, AFA36198.1, AFA36199.1, AFA36200.1, AFA36201.1, AFA36202.1, AFA36203.1, AFE62872.1, AFH78104.1, AFI23635.1, AFK50391.1, AFP86408.1, AGZ01906.1, AIR95840.1, AJE59989.1, AJE60060.1, AJE60131.1, AJE60202.1, AKE48623.1, AKE98415.1, AKE98416.1, AKE98417.1, AKE98418.1, AKE98419.1, AKE98420.1, AKE98421.1, AKE98422.1, AKE98423.1, AKE98424.1, AKE98425.1, AKE98426.1, AKE98427.1, AKE98428.1, AKE98429.1, AKE98430.1, AKE98431.1, AKE98432.1, AKE98433.1, AKE98434.1, AKE98435.1, AKG59227.1, AKG59299.1, AKG59372.1, AKG59444.1, AKG59516.1, AKG59591.1, AKG59663.1, AKG59736.1, AKG59807.1, AKG59879.1, AKG59953.1, AKG60027.1, AKG60099.1, AKG60170.1, AKG60243.1, AKG60316.1, AKG60386.1, AKG60456.1, AKG60528.1, AKG60601.1, AKG60674.1, AKG60745.1, AKG60817.1, AKG60887.1, AKG60959.1, AKG61032.1, AKG61104.1, AKG61175.1, AKG61248.1, AKG61321.1, AKG61392.1, AKG61464.1, AKG61537.1, AKG61611.1, AKG61684.1, AKG61756.1, AKG61828.1, AKG61902.1, AKG61974.1, AKH80444.1, AKH80517.1, AKM76368.1, ALM22613.1, ALM22687.1, ALM22761.1, ALM22835.1, ALO18641.1, ALO18717.1, AMB65642.1, AMB65715.1, AMB65862.1, AMN09813.1, ANN83942.1, ANN84019.1, ANN84095.1, ANN84172.1, ANN84249.1, ANN84326.1, ANN84403.1, ANN84478.1, ANN84555.1, ANN84632.1, ANN84708.1, ANN84785.1, ANN84861.1, ANN84938.1, ANN85014.1, ANN85091.1, ANN85167.1, ANN85242.1, ANN85319.1, ANN85396.1, ANN85472.1, ANN85549.1, ANN85626.1, ANN85703.1, ANN85779.1, AOY34308.1, AOY36663.1, AOY36687.1, ARB08935.1, ARO38059.1, ARO38060.1, ARO38061.1, ARO38062.1, ARO38063.1, ARO38064.1, ARO38065.1, ARO38066.1, ASM47642.1, ASM47719.1, ASM47796.1, ASM47871.1, BAM73394.1, CAA32294.1, CAB40083.1, CAD13356.1, CAD13357.1, CAD13358.1, CAD13359.1, CAD13360.1, CAD13361.1, CAD13362.1, CAD13363.1, CAD13364.1, CAD13365.1, CAD13366.1, CAD13367.1, CAD13368.1, CAD13369.1, CAD13370.1, CAD13371.1, CAD13372.1, CAD13373.1, CAD13374.1, CAD13375.1, CAD13376.1, CAD13377.1, CAD13378.1, P04290.1, P04488.1, P09855.1, P10228.1, P28986.1, SBO07729.1, SBO07793.1, SBO07798.1, SBO07812.1, SBO07880.1, SBS69375.1, SBS69379.1, SBS69440.1, SBS69448.1, SBS69560.1, SBS69599.1, SBS69602.1, SBS69637.1, SBS69790.1, SBT69374.1, SCL76887.1, YP_009137119.1, or YP_009137143.1.

In another embodiment, the composition comprises a modified mRNA encoding an HSV-2 gC protein. In another embodiment, the composition comprises a modified mRNA encoding a fragment of an HSV-2 gC protein.

In one embodiment, the nucleotide sequence of the modified mRNA encoding an HSV-2 gC fragment comprises:

(SEQ ID NO: 10)
GGAAUAAAAGUCUCAACACAACAUAUACAAAACAAACGAAUCUCAAGCA
AUCAAGCAUUCUACUUCUAUUGCAGCAAUUUAAAUCAUUUCUUUUAAAG
CAAAAGCAAUUUUCUGAAAAUUUUCACCAUUUACGAACGAUAGCAUGCG
CAUGCAGCUGCUGCUGCUGAUCGCCCUGUCCCUGGCCCUGGUGACCAAC
UCCGCCUCCCCCGGCCGCACCAUCACCGUGGGCCCCCGCGGCAACGCCU
CCAACGCCGCCCCCUCCGCCUCCCCCCGCAACGCCUCCGCCCCCCGCAC
CACCCCCACCCCCCCCCAGCCCCGCAAGGCCACCAAGUCCAAGGCCUCC
ACCGCCAAGCCCGCCCCCCCCCCCAAGACCGGCCCCCCCAAGACCUCCU
CCGAGCCCGUGCGCUGCAACCGCCACGACCCCCUGGCCCGCUACGGCUC
CCGCGUGCAGAUCCGCUGCCGCUUCCCCAACUCCACCCGCACCGAGUUC
CGCCUGCAGAUCUGGCGCUACGCCACCGCCACCGACGCCGAGAUCGGCA
CCGCCCCCUCCCUGGAGGAGGUGAUGGUGAACGUGUCCGCCCCCCCCGG
CGGCCAGCUGGUGUACGACUCCGCCCCCAACCGCACCGACCCCCACGUG
AUCUGGGCCGAGGGCGCCGGCCCCGGCGCCUCCCCCCGCCUGUACUCCG
UGGUGGGCCCCCUGGGCCGCCAGCGCCUGAUCAUCGAGGAGCUGACCCU
GGAGACCCAGGGCAUGUACUACUGGGUGUGGGGCCGCACCGACCGCCCC
UCCGCCUACGGCACCUGGGUGCGCGUGCGCGUGUUCCGCCCCCCCUCCC
UGACCAUCCACCCCCACGCCGUGCUGGAGGGCCAGCCCUUCAAGGCCAC
CUGCACCGCCGCCACCUACUACCCCGGCAACCGCGCCGAGUUCGUGUGG
UUCGAGGACGGCCGCCGCGUGUUCGACCCCGCCCAGAUCCACACCCAGA
CCCAGGAGAACCCCGACGGCUUCUCCACCGUGUCCACCGUGACCUCCGC
CGCCGUGGGCGGCCAGGGCCCCCCCCGCACCUUCACCUGCCAGCUGACC
UGGCACCGCGACUCCGUGUCCUUCUCCCGCCGCAACGCCUCCGGCACCG
CCUCCGUGCUGCCCCGCCCCACCAUCACCAUGGAGUUCACCGGCGACCA
CGCCGUGUGCACCGCCGGCUGCGUGCCCGAGGGCGUGACCUUCGCCUGG
UUCCUGGGCGACGACUCCUCCCCCGCCGAGAAGGUGGCCGUGGCCUCCC
AGACCUCCUGCGGCCGCCCCGGCACCGCCACCAUCCGCUCCACCCUGCC
CGUGUCCUACGAGCAGACCGAGUACAUCUGCCGCCUGGCCGGCUACCCC
GACGGCAUCCCCGUGCUGGAGCACCACUAACUAGUAGUGACUGACUAGG
AUCUGGUUACCACUAAACCAGCCUCAAGAACACCCGAAUGGAGUCUCUA
AGCUACAUAAUACCAACUUACACUUACAAAAUGUUGUCCCCCAAAAUGU
AGCCAUUCGUAUCUGCUCCUAAUAAAAAGAAAGUUUCUUCACAUUCUAA
AAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAA
AC.

In one embodiment, all uridine residues are 1-methyl-pseudouridine. In one embodiment, underlined residues represent 5′ untranslated sequences. In one embodiment, bold residues represent a signal sequence (leader sequence) to assist expression of the gC2 fragment. In one embodiment, italicized residues represent 3′ untranslated sequences and poly adenylation tail.

In another embodiment, the nucleotide sequence of the modified mRNA encoding an HSV-2 gC fragment lacks the 5′ untranslated sequences, the signal sequence, the 3′ untranslated sequences, the poly adenylation tail, or a combination thereof.

In one embodiment, the HSV-2 gC fragment encoded by modified mRNA utilized in the methods and compositions of the present invention comprises amino acids 27-426 of gC from HSV-2 strain 333, as set forth in the following amino acid sequence:

(SEQ ID NO: 11)
ASPGRTITVGPRGNASNAAPSASPRNASAPRTTPTPPQPRKATKSKAST
AKPAPPPKTGPPKTSSEPVRCNRHDPLARYGSRVQIRCRFPNSTRTESR
LQIWRYATATDAEIGTAPSLEEVMVNVSAPPGGQLVYDSAPNRTDPHVI
WAEGAGPGASPRLYSVVGPLGRQRLIIEELTLETQGMYYWVWGRTDRPS
AYGTWVRVRVFRPPSLTIHPHAVLEGQPFKATCTAATYYPGNRAEFVWF
EDGRRVFDPAQIHTQTQENPDGFSTVSTVTSAAVGGQGPPRTFTCQLTW
HRDSVSFSRRNASGTASVLPRPTITMEFTGDHAVCTAGCVPEGVTFAWF
LGDDSSPAEKVAVASQTSCGRPGTATIRSTLPVSYEQTEYICRLAGYPD
GIPVLEHH.

In one embodiment, the full length HSV-2 gC encoded by modified mRNA utilized in the methods and compositions of the present invention comprises the following amino acid sequence:

(SEQ ID NO: 12)
MALGRVGLAVGLWGLLWVGVVVVLANASPGRTITVGPRGNASNAAPSAS
PRNASAPRTTPTPPQPRKATKSKASTAKPAPPPKTGPPKTSSEPVRCNR
HDPLARYGSRVQIRCRFPNSTRTEFRLQIWRYATATDAEIGTAPSLEEV
MVNVSAPPGGQLVYDSAPNRTDPHVIWAEGAGPGASPRLYSVVGPLGRQ
RLIIEELTLETQGMYYWVWGRTDRPSAYGTWVRVRVFRPPSLTIHPHAV
LEGQPFKATCTAATYYPGNRAEFVWFEDGRRVFDPAQIHTQTQENPDGF
STVSTVTSAAVGGQGPPRTFTCQLTWHRDSVSFSRRNASGTASVLPRPT
ITMEFTGDHAVCTAGCVPEGVTFAWFLGDDSSPAEKVAVASQTSCGRPG
TATIRSTLPVSYEQTEYICRLAGYPDGIPVLEHHGSHQPPPRDPTERQV
IRAVEGAGIGVAVLVAVVLAGTAVVYLTHASSVRYRRLR.

In another embodiment, the HSV-2 gC encoded by modified mRNA utilized in the methods and compositions of the present invention comprises the amino acid sequences as set forth in any of the following GenBank Accession Numbers: AAA20532.1, AAA66442.1, AAB60549.1, AAB60550.1, AAB60551.1, AAB72101.1, ABU45429.1, ABU45430.1, ABU45431.1, ABU45432.1, ABU45459.1, ABU45460.1, AEV91348.1, AEV91383.1, AEV91407.1, AFM93864.1, AHG54708.1, AKC42808.1, AKC59285.1, AKC59357.1, AKC59428.1, AKC59499.1, AKC59570.1, AMB66008.1, AMB66079.1, AMB66151.1, AMB66224.1, AMB66252.1, AMB66253.1, AMB66368.1, AMB66441.1, AQZ55735.2, AQZ55806.1, AQZ55877.1, AQZ55948.1, AQZ56019.1, AQZ56090.1, AQZ56161.2, AQZ56232.2, AQZ56303.2, AQZ56374.2, AQZ56445.1, AQZ56516.1, AQZ56587.1, AQZ56658.1, AQZ56729.2, AQZ56800.1, AQZ56871.1, AQZ56942.2, AQZ57013.1, AQZ57084.2, AQZ57155.1, AQZ57226.1, AQZ57297.1, AQZ57368.1, AQZ57439.1, AQZ57510.1, AQZ57581.1, AQZ57652.1, AQZ57723.1, AQZ57794.2, AQZ57865.2, AQZ57936.1, AQZ58007.2, AQZ58078.1, AQZ58149.2, AQZ58220.1, AQZ58291.1, AQZ58362.1, AQZ58433.1, AQZ58504.1, AQZ58575.1, AQZ58646.1, AQZ58717.2, AQZ58788.2, AQZ58859.2, AQZ58930.1, AQZ59001.2, AQZ59072.1, AQZ59143.1, ARO38067.1, ARO38068.1, ARO38069.1, ARO38070.1, ARO38071.1, ARO38072.1, CAA25687.1, CAA26025.1, CAB06730.1, CAB06734.1, CAB96544.1, P03173.1, P06475.1, P89475.1, Q89730.1, YP_009137161.1, YP_009137196.1, or YP_009137220.1.

In another embodiment, the gC protein fragment encoded by modified mRNA utilized in the methods and compositions of the present invention comprises a properdin interfering domain “Properdin-interfering domain” refers, in one embodiment, to a domain that blocks or inhibits binding of a host C3b molecule with a host properdin molecule. In another embodiment, the term refers to a domain that blocks or inhibits an interaction of a host C3b molecule with a host properdin molecule.

In another embodiment, the gC protein fragment encoded by modified mRNA utilized in the methods and compositions of the present invention is a C5 interfering domain. In another embodiment, the gC protein fragment is a portion of a C5 interfering domain. “C5-interfering domain” refers, in another embodiment, to a domain that interferes with binding of a host C3b molecule with a host C5 molecule. In another embodiment, the term refers to a domain that interferes with the interaction of a host C3b molecule with a host C5 molecule.

Each modified mRNA encoding gC-1 or gC-2 protein or fragment thereof represents a separate embodiment of the present invention.

In another embodiment, a gC protein fragment encoded by modified mRNA utilized in the methods and compositions of the present invention is an immunogenic fragment. In another embodiment, a gC immunoprotective antigen need not be the entire protein. The protective immune response generally involves, in another embodiment, an antibody response. In another embodiment, mutants, sequence conservative variants, and functional conservative variants of gC are useful in methods and compositions of the present invention, provided that all such variants retain the required immuno-protective effect. In another embodiment, the immunogenic fragment can comprise an immuno-protective gC antigen from any strain of HSV. In another embodiment, the immunogenic fragment can comprise sequence variants of HSV, as found in infected individuals.

### Glycoprotein E

In another embodiment, a composition of the present invention comprises a modified mRNA encoding HSV-1 gE protein. In another embodiment, the composition comprises a modified mRNA encoding a fragment of an HSV-1 gE protein.

In one embodiment, the nucleotide sequence of the modified mRNA encoding an HSV-1 gE fragment comprises:

(SEQ ID NO: 13)
GGAAUAAAAGUCUCAACACAACAUAUACAAAACAAACGAAUCUCAAGCA
AUCAAGCAUUCUACUUCUAUUGCAGCAAUUUAAAUCAUUUCUUUUAAAG
CAAAAGCAAUUUUCUGAAAAUUUUCACCAUUUACGAACGAUAGCAUGCG
CAUGCAGCUGCUGCUGCUGAUCGCCCUGUCCCUGGCCCUGGUGACCAAC
UCCAAGACCUCCUGGCGCCGCGUGUCCGUGGGCGAGGACGUGUCCCUGC
UGCCCGCCCCCGGCCCCACCGGCCGCGGCCCCACCCAGAAGCUGCUGUG
GGCCGUGGAGCCCCUGGACGGCUGCGGCCCCCUGCACCCCUCCUGGGUG
UCCCUGAUGCCCCCCAAGCAGGUGCCCGAGACCGUGGUGGACGCCGCCU
GCAUGCGCGCCCCCGUGCCCCUGGCCAUGGCCUACGCCCCCCCCGCCCC
CUCCGCCACCGGCGGCCUGCGCACCGACUUCGUGUGGCAGGAGCGCGCC
GCCGUGGUGAACCGCUCCCUGGUGAUCUACGGCGUGCGCGAGACCGACU
CCGGCCUGUACACCCUGUCCGUGGGCGACAUCAAGGACCCCGCCCGCCA
GGUGGCCUCCGUGGUGCUGGUGGUGCAGCCCGCCCCCGUGCCCACCCCC
CCCCCCACCCCCGCCGACUACGACGAGGACGACAACGACGAGGGCGAGG
GCGAGGACGAGUCCCUGGCCGGCACCCCCGCCUCCGGCACCCCCCGCCU
GCCCCCCUCCCCCGCCCCCCCCCGCUCCUGGCCCUCCGCCCCCGAGGUG
UCCCACGUGCGCGGCGUGACCGUGCGCAUGGAGACCCCCGAGGCCAUCC
UGUUCUCCCCCGGCGAGGCCUUCUCCACCAACGUGUCCAUCCACGCCAU
CGCCCACGACGACCAGACCUACACCAUGGACGUGGUGUGGCUGCGCUUC
GACGUGCCCACCUCCUGCGCCGAGAUGCGCAUCUACGAGUCCUGCCUGU
ACCACCCCCAGCUGCCCGAGUGCCUGUCCCCCGCCGACGCCCCCUGCGC
CGCCUCCACCUGGACCUCCCGCCUGGCCGUGCGCUCCUACGCCGGCUGC
UCCCGCACCAACCCCCCCCCCCGCUGCUCCGCCGAGGCCCACAUGGAGC
CCUUCCCCGGCCUGGCCUGGCAGGCCGCCUCCGUGAACCUGGAGUUCCG
CGACGCCUCCCCCCAGCACUCCGGCCUGUACCUGUGCGUGGUGUACGUG
AACGACCACAUCCACGCCUGGGGCCACAUCACCAUCAACACCGCCGCCC
AGUACCGCAACGCCGUGGUGGAGCAGCCCCUGCCCCAGCGCGGCGCCGA
CCUGGCCGAGCCCACCCACCCCCACGUGGGCGCCUAACUAGUAGUGACU
GACUAGGAUCUGGUUACCACUAAACCAGCCUCAAGAACACCCGAAUGGA
GUCUCUAAGCUACAUAAUACCAACUUACACUUACAAAAUGUUGUCCCCC
AAAAUGUAGCCAUUCGUAUCUGCUCCUAAUAAAAAGAAAGUUUCUUCAC
AUUCUAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAA
AAAAAAAAC.

In one embodiment, all uridine residues are 1-methyl-pseudouridine. In one embodiment, underlined residues represent 5′ untranslated sequences. In one embodiment, bold residues represent a signal sequence (leader sequence) to assist expression of the gE1 fragment. In one embodiment, italicized residues represent 3′ untranslated sequences and poly adenylation tail.

In another embodiment, the nucleotide sequence of the modified mRNA encoding an HSV-1 gE fragment lacks the 5′ untranslated sequences, the signal sequence, the 3′ untranslated sequences, the poly adenylation tail, or a combination thereof.

In one embodiment, the HSV-1 gE fragment encoded by modified mRNA utilized in the methods and compositions of the present invention comprises amino acids 24-409 of gE from HSV-1 NS strain, as set forth in the following amino acid sequence:

(SEQ ID NO: 14)
KTSWRRVSVGEDVSLLPAPGPTGRGPTQKLLWAVEPLDGCGPLHPSWVS
LMPPKQVPETVVDAACMRAPVPLAMAYAPPAPSATGGLRTDFVWQERAA
VVNRSLVIYGVRETDSGLYTLSVGDIKDPARQVASVVLVVQPAPVPTPP
PTPADYDEDDNDEGEGEDESLAGTPASGTPRLPPSPAPPRSWPSAPEVS
HVRGVTVRMETPEAILFSPGEAFSTNVSIHAIAHDDQTYTMDVVWLRFD
VPTSCAEMRIYESCLYHPQLPECLSPADAPCAASTWTSRLAVRSYAGCS
RTNPPPRCSAEAHMEPFPGLAWQAASVNLEFRDASPQHSGLYLCVVYVN
DHIHAWGHITINTAAQYRNAVVEQPLPQRGADLAEPTHPHVGA.

In one embodiment, the gE fragment encoded by modified mRNA utilized in the methods and compositions of the present invention comprises amino acids 24-409 of gE from an HSV-1 strain.

In one embodiment, the full length HSV-1 gE encoded by modified mRNA utilized in the methods and compositions of the present invention comprises the following amino acid sequence:

(SEQ ID NO: 15)
MDRGAVVGFLLGVCVVSCLAGTPKTSWRRVSVGEDVSLLPAPGPTGRGP
TQKLLWAVEPLDGCGPLHPSWVSLMPPKQVPETVVDAACMRAPVPLAMA
YAPPAPSATGGLRTDFVWQERAAVVNRSLVIYGVRETDSGLYTLSVGDI
KDPARQVASVVLVVQPAPVPTPPPTPADYDEDDNDEGEGEDESLAGTPA
SGTPRLPPSPAPPRSWPSAPEVSHVRGVTVRMETPEAILFSPGEAFSTN
VSIHAIAHDDQTYTMDVVWLRFDVPTSCAEMRIYESCLYHPQLPECLSP
ADAPCAASTWTSRLAVRSYAGCSRTNPPPRCSAEAHMEPFPGLAWQAAS
VNLEFRDASPQHSGLYLCVVYVNDHIHAWGHITINTAAQYRNAVVEQPL
PQRGADLAEPTHPHVGAPPHAPPTHGALRLGAVMGAALLLSALGLSVWA
CMTCWRRRAWRAVKSRASGKGPTYIRVADSELYADWSSDSEGERDQVPW
LAPPERPDSPSTNGSGFEILSPTAPSVYPRSDGHQSRRQLTTFGSGRPD
RRYSQASDSSVFW.

In another embodiment, the HSV-1 gE encoded by modified mRNA utilized in the methods and compositions of the present invention comprises the amino acid sequences as set forth in any of the following GenBank Accession Numbers: AAA45779.1, AAA96680.1, ABI63526.1, ACM62297.1, ADD60055.1, ADD60132.1, ADM22391.1, ADM22468.1, ADM22544.1, ADM22621.1, ADM22698.1, ADM22775.1, ADM22851.1, ADM22928.1, ADM23005.1, ADM23081.1, ADM23157.1, ADM23233.1, ADM23311.1, ADM23385.1, ADM23459.1, ADM23533.1, ADM23607.1, ADM23682.1, ADM23757.1, ADM23833.1, ADN34689.1, ADN34692.1, ADN34695.1, AEQ77099.1, AER37649.1, AER37717.1, AER37788.1, AER37859.1, AER37931.1, AER38002.1, AER38072.1, AFA36179.1, AFA36180.1, AFA36181.1, AFA36182.1, AFA36183.1, AFA36184.1, AFA36185.1, AFA36186.1, AFA36187.1, AFA36188.1, AFA36189.1, AFA36190.1, AFA36191.1, AFA36192.1, AFA36193.1, AFA36194.1, AFA36195.1, AFA36196.1, AFA36197.1, AFA36198.1, AFA36199.1, AFA36200.1, AFA36201.1, AFA36202.1, AFA36203.1, AFE62896.1, AFI23659.1, AFK50417.1, AFP86432.1, AGZ01930.1, AIR95859.1, AJE60011.1, AJE60082.1, AJE60153.1, AJE60224.1, AJE60295.1, AKE48647.1, AKE98373.1, AKE98374.1, AKE98375.1, AKE98376.1, AKE98377.1, AKE98378.1, AKE98379.1, AKE98380.1, AKE98381.1, AKE98382.1, AKE98383.1, AKE98384.1, AKE98385.1, AKE98386.1, AKE98387.1, AKE98388.1, AKE98389.1, AKE98390.1, AKE98391.1, AKE98392.1, AKE98393.1, AKG59248.1, AKG59320.1, AKG59393.1, AKG59464.1, AKG59538.1, AKG59611.1, AKG59684.1, AKG59757.1, AKG59828.1, AKG59900.1, AKG59974.1, AKG60048.1, AKG60120.1, AKG60191.1, AKG60263.1, AKG60336.1, AKG60406.1, AKG60476.1, AKG60548.1, AKG60622.1, AKG60694.1, AKG60765.1, AKG60837.1, AKG60908.1, AKG60980.1, AKG61052.1, AKG61125.1, AKG61196.1, AKG61269.1, AKG61341.1, AKG61413.1, AKG61486.1, AKG61558.1, AKG61631.1, AKG61705.1, AKG61776.1, AKG61849.1, AKG61922.1, AKG61995.1, AKH80465.1, AKH80538.1, ALM22637.1, ALM22711.1, ALM22785.1, ALM22859.1, ALO18664.1, ALO18740.1, AMB65664.1, AMB65737.1, AMB65811.1, AMB65887.1, AMB65958.1, AMN09834.1, ANN83966.1, ANN84043.1, ANN84119.1, ANN84196.1, ANN84273.1, ANN84350.1, ANN84426.1, ANN84502.1, ANN84579.1, ANN84655.1, ANN84732.1, ANN84808.1, ANN84885.1, ANN84961.1, ANN85038.1, ANN85114.1, ANN85189.1, ANN85266.1, ANN85343.1, ANN85418.1, ANN85496.1, ANN85573.1, ANN85650.1, ANN85726.1, ANN85803.1, AOY34085.1, AOY36687.1, ARB08959.1, ARO38073.1, ARO38074.1, ARO38075.1, ARO38076.1, ARO38077.1, ARO38078.1, ARO38079.1, ARO38080.1, ASM47642.1, ASM47666.1, ASM47743.1, ASM47820.1, ASM47895.1, BAM73421.1, CAA26062.1, CAA32272.1, CAF24756.1, CAF24757.1, CAF24758.1, CAF24759.1, CAF24760.1, CAF24761.1, CAF24762.1, CAF24763.1, CAF24764.1, CAF24765.1, CAF24766.1, CAF24767.1, CAF24768.1, CAF24769.1, CAF24770.1, CAF24771.1, CAF24772.1, CAF24773.1, CAF24774.1, CAF24775.1, CAF24776.1, CAF24777.1, CAF24778.1, CAF24779.1, CAF24780.1, CAF24781.1, CAF24782.1, CAF24783.1, CAF24784.1, CAF24785.1, P04290.1, P04488.1, P28986.1, Q703F0.1, SBO07910.1, SBS69571.1, SBS69576.1, SBS69595.1, SBS69636.1, SBS69693.1, SBS69701.1, SBS69722.1, SBS69732.1, SBS69813.1, SBT69397.1, or YP_009137143.1.

In another embodiment, the composition comprises a modified mRNA encoding an HSV-2 gE protein. In another embodiment, the composition comprises a modified mRNA encoding a fragment of an HSV-2 gE protein.

In one embodiment, the nucleotide sequence of the modified mRNA encoding an HSV-2 gE fragment comprises:

(SEQ ID NO: 16)
GGAAUAAAAGUCUCAACACAACAUAUACAAAACAAACGAAUCUCAAGCA
AUCAAGCAUUCUACUUCUAUUGCAGCAAUUUAAAUCAUUUCUUUUAAAG
CAAAAGCAAUUUUCUGAAAAUUUUCACCAUUUACGAACGAUAGCAUGCG
CAUGCAGCUGCUGCUGCUGAUCGCCCUGUCCCUGGCCCUGGUGACCAAC
UCCCGCACCUCCUGGAAGCGCGUGACCUCCGGCGAGGACGUGGUGCUGC
UGCCCGCCCCCGCCGGCCCCGAGGAGCGCACCCGCGCCCACAAGCUGCU
GUGGGCCGCCGAGCCCCUGGACGCCUGCGGCCCCCUGCGCCCCUCCUGG
GUGGCCCUGUGGCCCCCCCGCCGCGUGCUGGAGACCGUGGUGGACGCCG
CCUGCAUGCGCGCCCCCGAGCCCCUGGCCAUCGCCUACUCCCCCCCCUU
CCCCGCCGGCGACGAGGGCCUGUACUCCGAGCUGGCCUGGCGCGACCGC
GUGGCCGUGGUGAACGAGUCCCUGGUGAUCUACGGCGCCCUGGAGACCG
ACUCCGGCCUGUACACCCUGUCCGUGGUGGGCCUGUCCGACGAGGCCCG
CCAGGUGGCCUCCGUGGUGCUGGUGGUGGAGCCCGCCCCCGUGCCCACC
CCCACCCCCGACGACUACGACGAGGAGGACGACGCCGGCGUGUCCGAGC
GCACCCCCGUGUCCGUGCCCCCCCCCACCCCCCCCCGCCGCCCCCCCGU
GGCCCCCCCCACCCACCCCCGCGUGAUCCCCGAGGUGUCCCACGUGCGC
GGCGUGACCGUGCACAUGGAGACCCCCGAGGCCAUCCUGUUCGCCCCCG
GCGAGACCUUCGGCACCAACGUGUCCAUCCACGCCAUCGCCCACGACGA
CGGCCCCUACGCCAUGGACGUGGUGUGGAUGCGCUUCGACGUGCCCUCC
UCCUGCGCCGAGAUGCGCAUCUACGAGGCCUGCCUGUACCACCCCCAGC
UGCCCGAGUGCCUGUCCCCCGCCGACGCCCCCUGCGCCGUGUCCUCCUG
GGCCUACCGCCUGGCCGUGCGCUCCUACGCCGGCUGCUCCCGCACCACC
CCCCCCCCCCGCUGCUUCGCCGAGGCCCGCAUGGAGCCCGUGCCCGGCC
UGGCCUGGCUGGCCUCCACCGUGAACCUGGAGUUCCAGCACGCCUCCCC
CCAGCACGCCGGCCUGUACCUGUGCGUGGUGUACGUGGACGACCACAUC
CACGCCUGGGGCCACAUGACCAUCUCCACCGCCGCCCAGUACCGCAACG
CCGUGGUGGAGCAGCACCUGCCCCAGCGCCAGCCCGAGCCCGUGGAGCC
CACCCGCCCCCACGUGCGCGCCUAACUAGUAGUGACUGACUAGGAUCUG
GUUACCACUAAACCAGCCUCAAGAACACCCGAAUGGAGUCUCUAAGCUA
CAUAAUACCAACUUACACUUACAAAAUGUUGUCCCCCAAAAUGUAGCCA
UUCGUAUCUGCUCCUAAUAAAAAGAAAGUUUCUUCACAUUCUAAAAAAA
AAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAC

In one embodiment, all uridine residues are 1-methyl-pseudouridine. In one embodiment, underlined residues represent 5′ untranslated sequences. In one embodiment, bold residues represent a signal sequence (leader sequence) to assist expression of the gE2 fragment. In one embodiment, italicized residues represent 3′ untranslated sequences and poly adenylation tail.

In another embodiment, the nucleotide sequence of the modified mRNA encoding an HSV-2 gE fragment lacks the 5′ untranslated sequences, the signal sequence, the 3′ untranslated sequences, the poly adenylation tail, or a combination thereof.

In one embodiment, the HSV-2 gE fragment encoded by modified mRNA utilized in the methods and compositions of the present invention comprises amino acids 24-405 of gE from HSV-2 strain 2.12 as set forth in the following amino acid sequence:

(SEQ ID NO: 17)
RTSWKRVTSGEDVVLLPAPAGPEERTRAHKLLWAAEPLDACGPLRPSWV
ALWPPRRVLETVVDAACMRAPEPLAIAYSPPFPAGDEGLYSELAWRDRV
AVVNESLVIYGALETDSGLYTLSVVGLSDEARQVASVVLVVEPAPVPTP
TPDDYDEEDDAGVSERTPVSVPPPTPPRRPPVAPPTHPRVIPEVSHVRG
VTVHMETPEAILFAPGETFGTNVSIHAIAHDDGPYAMDVVWMRFDVPSS
CAEMRIYEACLYHPQLPECLSPADAPCAVSSWAYRLAVRSYAGCSRTTP
PPRCFAEARMEPVPGLAWLASTVNLEFQHASPQHAGLYLCVVYVDDHIH
AWGHMTISTAAQYRNAVVEQHLPQRQPEPVEPTRPHVRA.

In one embodiment, the full length HSV-2 gE encoded by modified mRNA utilized in the methods and compositions of the present invention comprises the following amino acid sequence:

(SEQ ID NO: 18)
MARGAGLVFFVGVWVVSCLAAAPRTSWKRVTSGEDVVLLPAPAERTRAH
KLLWAAEPLDACGPLRPSWVALWPPRRVLETVVDAACMRAPEPLAIAYS
PPFPAGDEGLYSELAWRDRVAVVNESLVIYGALETDSGLYTLSVVGLSD
EARQVASVVLVVEPAPVPTPTPDDYDEEDDAGVTNARRSAFPPQPPPRR
PPVAPPTHPRVIPEVSHVRGVTVHMETLEAILFAPGETFGTNVSIHAIA
HDDGPYAMDVVWMRFDVPSSCADMRIYEACLYHPQLPECLSPADAPCAV
SSWAYRLAVRSYAGCSRTTPPPRCFAEARMEPVPGLAWLASTVNLEFQH
ASPQHAGLYLCVVYVDDHIHAWGHMTISTAAQYRNAVVEQHLPQRQPEP
VEPTRPHVRAPHPAPSARGPLRLGAVLGAALLLAALGLSAWACMTCWRR
RSWRAVKSRASATGPTYIRVADSELYADWSSDSEGERDGSLWQDPPERP
DSPSTNGSGFEILSPTAPSVYPHSEGRKSRRPLTTFGSGSPGRRHSQAS
YPSVLW.

In another embodiment, the HSV-2 gE encoded by modified mRNA utilized in the methods and compositions of the present invention comprises the amino acid sequences as set forth in any of the following GenBank Accession Numbers: ABU45436.1, ABU45437.1, ABU45438.1, ABU45439.1, ABW83306.1, ABW83308.1, ABW83310.1, ABW83312.1, ABW83314.1, ABW83316.1, ABW83318.1, ABW83320.1, ABW83322.1, ABW83324.1, ABW83326.1, ABW83328.1, ABW83330.1, ABW83332.1, ABW83334.1, ABW83336.1, ABW83338.1, ABW83340.1, ABW83342.1, ABW83344.1, ABW83346.1, ABW83348.1, ABW83350.1, ABW83352.1, ABW83354.1, ABW83356.1, ABW83358.1, ABW83360.1, ABW83362.1, ABW83364.1, ABW83366.1, ABW83368.1, ABW83370.1, ABW83372.1, ABW83374.1, ABW83376.1, ABW83378.1, ABW83380.1, ABW83382.1, ABW83384.1, ABW83386.1, ABW83388.1, ABW83390.1, ABW83392.1, ABW83394.1, ABW83396.1, ABW83398.1, ABW83400.1, ABZ04069.1, AEV91407.1, AHG54732.1, AKC42830.1, AKC59307.1, AKC59378.1, AKC59449.1, AKC59520.1, AKC59591.1, AMB66104.1, AMB66173.1, AMB66246.1, AMB66465.1, AQZ55756.1, AQZ55827.1, AQZ55898.1, AQZ55969.2, AQZ56040.2, AQZ56111.2, AQZ56182.1, AQZ56253.2, AQZ56324.1, AQZ56395.1, AQZ56466.2, AQZ56537.1, AQZ56608.1, AQZ56679.1, AQZ56750.1, AQZ56821.2, AQZ56892.1, AQZ56963.2, AQZ57034.2, AQZ57105.1, AQZ57176.1, AQZ57247.2, AQZ57318.2, AQZ57389.2, AQZ57460.2, AQZ57531.2, AQZ57602.2, AQZ57673.1, AQZ57744.2, AQZ57815.1, AQZ57886.1, AQZ57957.2, AQZ58028.2, AQZ58099.1, AQZ58170.2, AQZ58241.2, AQZ58312.2, AQZ58383.2, AQZ58454.2, AQZ58525.2, AQZ58596.1, AQZ58667.1, AQZ58738.2, AQZ58809.2, AQZ58880.2, AQZ58951.2, AQZ59022.2, AQZ59093.1, AQZ59164.1, ARO38081.1, ARO38082.1, ARO38083.1, ARO38084.1, ARO38085.1, ARO38086.1, CAB06715.1, P89436.1, P89475.1, or YP_009137220.1.

In another embodiment, a gE fragment encoded by modified mRNA utilized in the methods and compositions of the present invention comprises an IgG Fc-binding domain of the gE protein. In another embodiment, the gE domain encoded by modified mRNA utilized in the methods and compositions of the present invention is any other gE domain known in the art to mediate binding to IgG Fc.

In another embodiment, the gE protein encoded by modified mRNA utilized in the methods and compositions of the present invention comprises a gE domain involved in cell-to-cell spread.

In another embodiment, the gE fragment encoded by modified mRNA fragment utilized in the methods and compositions of the present invention comprises an immune evasion domain. In another embodiment, the gE fragment encoded by modified mRNA fragment utilized in the methods and compositions of the present invention comprises a portion of an immune evasion domain.

Each modified mRNA encoding gE-1 or gE-2 protein or fragment thereof represents a separate embodiment of the present invention.

In another embodiment, a gE protein fragment encoded by modified mRNA utilized in the methods and compositions of the present invention is an immunogenic fragment. In another embodiment, a gE immunoprotective antigen need not be the entire protein. The protective immune response generally involves, in another embodiment, an antibody response.

In another embodiment, mutants, sequence conservative variants, and functional conservative variants of gE are useful in methods and compositions of the present invention, provided that all such variants retain the required immuno-protective effect. In another embodiment, the immunogenic fragment can comprise an immuno-protective gE antigen from any strain of HSV. In another embodiment, the immunogenic fragment can comprise sequence variants of HSV, as found in infected individuals.

In one embodiment, an HSV glycoprotein encoded by modified mRNA utilized in the methods and compositions of the present invention is a homologue of the sequence provided herein. In another embodiment, an HSV glycoprotein encoded by modified mRNA utilized in the methods and compositions of the present invention is an isoform of the sequence provided herein. In another embodiment, an HSV glycoprotein encoded by modified mRNA utilized in the methods and compositions of the present invention is a variant of the sequence provided herein. In another embodiment, an HSV glycoprotein encoded by modified mRNA utilized in the methods and compositions of the present invention is a fragment of the sequence provided herein.

In another embodiment, the glycoprotein fragment encoded by modified mRNA of the methods and compositions of the present invention comprises the ectodomain of the glycoprotein. In another embodiment, the glycoprotein fragment encoded by modified mRNA of the methods and compositions of the present invention consists of the ectodomain of the glycoprotein. In another embodiment, the glycoprotein fragment encoded by modified mRNA of the methods and compositions of the present invention comprises a fragment of the ectodomain of the glycoprotein. In another embodiment, the glycoprotein fragment may be any glycoprotein fragment known in the art.

In another embodiment, the glycoprotein or immunogenic fragment encoded by modified mRNA fragment utilized in the methods and compositions of the present invention may be from any strain of HSV. In another embodiment, the immunogenic fragment encoded by modified mRNA fragment utilized in the methods and compositions of the present invention may comprise sequence variants of HSV, as found in infected individuals.

In one embodiment, “variant” refers to an amino acid or nucleic acid sequence (or in other embodiments, an organism or tissue) that is different from the majority of the population but is still sufficiently similar to the common mode to be considered to be one of them, for example splice variants. In one embodiment, the variant may a sequence conservative variant, while in another embodiment, the variant may be a functional conservative variant. In one embodiment, a variant may comprise an addition, deletion or substitution of one or more amino acids.

“Immune evasion domain” refers, in one embodiment, to a domain that interferes with or reduces in vivo anti-HSV efficacy of anti-HSV antibodies (e.g. anti-gD antibodies). In another embodiment, the domain interferes or reduces in vivo anti-HSV efficacy of an anti-HSV immune response. In another embodiment, the domain reduces the immunogenicity of an HSV protein (e.g. gD) during subsequent infection. In another embodiment, the domain reduces the immunogenicity of an HSV protein during subsequent challenge. In another embodiment, the domain reduces the immunogenicity of HSV during subsequent challenge. In another embodiment, the domain reduces the immunogenicity of an HSV protein in the context of ongoing HSV infection. In another embodiment, the domain reduces the immunogenicity of HSV in the context of ongoing HSV infection. In another embodiment, the domain functions as an IgG Fc receptor. In another embodiment, the domain promotes antibody bipolar bridging, which in one embodiment, is a term that refers to an antibody molecule binding by its Fab domain to an HSV antigen and by its Fc domain to a separate HSV antigen, such as in one embodiment, gE, thereby blocking the ability of the Fc domain to activate complement.

The present invention also provides for modified mRNA encoding analogs of HSV proteins or polypeptides, or fragments thereof. Analogs may differ from naturally occurring proteins or peptides by conservative amino acid sequence substitutions or by modifications which do not affect sequence, or by both.

In another embodiment, an HSV glycoprotein encoded by modified mRNA of the present invention is homologous to a sequence set forth hereinabove, either expressly or by reference to a GenBank entry. The terms “homology,” “homologous,” etc., when in reference to any protein or peptide, refer, in one embodiment, to a percentage of amino acid residues in the candidate sequence that are identical with the residues of a corresponding native polypeptide, after aligning the sequences and introducing gaps, if necessary, to achieve the maximum percent homology, and not considering any conservative substitutions as part of the sequence identity. Methods and computer programs for the alignment are well known in the art.

In another embodiment, “homology” refers to identity of a protein sequence encoded by a modified mRNA to a sequence disclosed herein of greater than 70%. In another embodiment, the identity is greater than 72%. In another embodiment, the identity is greater than 75%. In another embodiment, the identity is greater than 78%. In another embodiment, the identity is greater than 80%. In another embodiment, the identity is greater than 82%. In another embodiment, the identity is greater than 83%. In another embodiment, the identity is greater than 85%. In another embodiment, the identity is greater than 87%. In another embodiment, the identity is greater than 88%. In another embodiment, the identity is greater than 90%. In another embodiment, the identity is greater than 92%. In another embodiment, the identity is greater than 93%. In another embodiment, the identity is greater than 95%. In another embodiment, the identity is greater than 96%. In another embodiment, the identity is greater than 97%. In another embodiment, the identity is greater than 98%. In another embodiment, the identity is greater than 99%. In another embodiment, the identity is 100%.

In one embodiment, “isoform” refers to aversion of a molecule, for example, a protein, with only slight differences to another isoform of the same protein. In one embodiment, isoforms may be produced from different but related genes, or in another embodiment, may arise from the same gene by alternative splicing. In another embodiment, isoforms are caused by single nucleotide polymorphisms.

In another embodiment, the modified mRNA encoding a glycoprotein or glycoprotein fragment as described herein further encodes an antigenic tag. In one embodiment, the tag is a histidine (“His”) tag. In one embodiment, the His tag comprises 5 histidine residues. In another embodiment, the His tag comprises 6 histidine residues.

In another embodiment, methods and compositions of the present invention utilize a chimeric molecule, comprising a fusion of a modified mRNA encoding an HSV protein with a modified mRNA encoding a tag polypeptide that provides an epitope to which an anti-tag antibody can selectively bind. The epitope tag is placed, in other embodiments, at the amino- or carboxyl-terminus of the protein or in an internal location therein. The presence of such epitope-tagged forms of the recombinant HSV protein is detected, in another embodiment, using an antibody against the tag polypeptide. In another embodiment, inclusion of the epitope tag enables the recombinant HSV protein to be readily purified by affinity purification using an anti-tag antibody or another type of affinity matrix that binds to the epitope tag. Various tag polypeptides and their respective antibodies are known in the art.

In one embodiment, the compositions of the present invention comprise an adjuvant, while in another embodiment, the compositions do not comprise an adjuvant. “Adjuvant” refers, in another embodiment, to compounds that, when administered to an individual or tested in vitro, increase the immune response to an antigen in the individual or test system to which the antigen is administered. In another embodiment, an immune adjuvant enhances an immune response to an antigen that is weakly immunogenic when administered alone, i.e., inducing no or weak antibody titers or cell-mediated immune response. In another embodiment, the adjuvant increases antibody titers to the antigen. In another embodiment, the adjuvant lowers the dose of the antigen effective to achieve an immune response in the individual. Multiple types of adjuvants are known in the art and described in detail in U. S. Patent Publication 2013/0028925 which is hereby incorporated by reference herein.

Modified mRNAs

In one embodiment, the present invention provides compositions comprising modified mRNAs and methods of use thereof. In one embodiment, the modified mRNA comprises one or more modified nucleoside residues.

In another embodiment, the modified nucleoside of the methods and compositions of the present invention is m5C (5-methylcytidine). In another embodiment, the modified nucleoside is m5U (5-methyluridine). In another embodiment, the modified nucleoside is m6A (N6-methyladenosine). In another embodiment, the modified nucleoside is s2U (2-thiouridine). In another embodiment, the modified nucleoside is Ψ (pseudouridine). In another embodiment, the modified nucleoside is Um (2′-O-methyluridine).

In other embodiments, the modified nucleoside is m¹A (1-methyladenosine), m²A (2-methyladenosine), m⁶A (N6-methyladenosine), Am (2′-O-methyladenosine), ms²m⁶A (2-methylthio-N6-methyladenosine), i⁶A (N6-isopentenyladenosine), ms²i⁶A (2-methylthio-N6-isopentenyladenosine), io⁶A (N6-(cis-hydroxyisopentenyl)adenosine), ms²io⁶A (2-methylthio-N6-(cis-hydroxyisopentenyl) adenosine), g⁶A (N6-glycinylcarbamoyladenosine), t⁶A (N6-threonylcarbamoyladenosine), ms²t⁶A (2-methylthio-N6-threonyl carbamoyladenosine), m⁶t⁶A (N6-methyl-N6-threonylcarbamoyladenosine), hn⁶A (N6-hydroxynorvalylcarbamoyladenosine), ms² hn⁶A (2-methylthio-N6-hydroxynorvalyl carbamoyladenosine), Ar(p) (2′-O-ribosyladenosine (phosphate)), I (inosine), m¹I (1-methylinosine), m¹Im (1,2′-O-dimethylinosine), m³C (3-methylcytidine), m⁵C (5-methylcytidine), Cm (2′-O-methylcytidine), s²C (2-thiocytidine), ac⁴C (N4-acetylcytidine), f⁵C (5-formylcytidine), m⁵Cm (5,2′-O-dimethylcytidine), ac⁴Cm (N4-acetyl-2′-O-methylcytidine), k²C (lysidine), m¹G (1-methylguanosine), m²G (N2-methylguanosine), m⁷G (7-methylguanosine), Gm (2′-O-methylguanosine), m²₂G (N2,N2-dimethylguanosine), m²Gm (N2,2′-O-dimethylguanosine), m²₂Gm (N2,N2,2′-O-trimethylguanosine), Gr(p) (2′-O-ribosylguanosine (phosphate)), yW (wybutosine), o₂yW (peroxywybutosine), OHyW (hydroxywybutosine), OHyW* (undermodified hydroxywybutosine), imG (wyosine), mimG (methylwyosine), Q (queuosine), oQ (epoxyqueuosine), galQ (galactosyl-queuosine), manQ (mannosyl-queuosine), preQ0 (7-cyano-7-deazaguanosine), preQ1 (7-aminomethyl-7-deazaguanosine), G⁺ (archaeosine), □ (pseudouridine), D (dihydrouridine), m⁵U (5-methyluridine), Um (2′-O-methyluridine), m⁵Um (5,2′-O-dimethyluridine), m¹□ (1-methylpseudouridine), □m (2′-O-methylpseudouridine), s²U (2-thiouridine), s⁴U (4-thiouridine), m⁵s²U (5-methyl-2-thiouridine), s²Um (2-thio-2′-O-methyluridine), acp³U (3-(3-amino-3-carboxypropyl)uridine), ho⁵U (5-hydroxyuridine), mo⁵U (5-methoxyuridine), cmo⁵U (uridine 5-oxyacetic acid), mcmo⁵U (uridine 5-oxyacetic acid methyl ester), chm⁵U (5-(carboxyhydroxymethyl)uridine), mchm⁵U (5-(carboxyhydroxymethyl)uridine methyl ester), mcm⁵U (5-methoxycarbonylmethyluridine), mcm⁵Um (5-methoxycarbonylmethyl-2′-O-methyluridine), mcm⁵s²U (5-methoxycarbonylmethyl-2-thiouridine), nm⁵s²U (5-aminomethyl-2-thiouridine), mnm⁵U (5-methylaminomethyluridine), mnm⁵s²U (5-methylaminomethyl-2-thiouridine), mnm⁵se²U (5-methylaminomethyl-2-selenouridine), ncm⁵U (5-carbamoylmethyluridine), nCm⁵Um (5-carbamoylmethyl-2′-O-methyluridine), cmnm⁵U (5-carboxymethylaminomethyluridine), cmnm⁵Um (5-carboxymethylaminomethyl-2′-O-methyluridine), cmnm⁵s²U (5-carboxymethylaminomethyl-2-thiouridine), m⁶₂A (N6,N6-dimethyladenosine), Im (2′-O-methylinosine), m⁴C (N4-methylcytidine), m⁴Cm (N4,2′-O-dimethylcytidine), hm⁵C (5-hydroxymethylcytidine), m³U (3-methyluridine), m¹acp³□ (1-methyl-3-(3-amino-3-carboxypropyl) pseudouridine), cm⁵U (5-carboxymethyluridine), m⁶Am (N6,2′-O-dimethyladenosine), m⁶²Am (N6,N6,2′-O-trimethyladenosine), m²,7G (N2,7-dimethylguanosine), m^{2,2,7}G (N2,N2,7-trimethylguanosine), m³Um (3,2′-O-dimethyluridine), m⁵D (5-methyldihydrouridine), m³□ (3-methylpseudouridine), f⁵Cm (5-formyl-2′-O-methylcytidine), m¹Gm (1,2′-O-dimethylguanosine), m¹Am (1,2′-O-dimethyladenosine), □m⁵U (5-taurinomethyluridine), □m⁵s²U (5-taurinomethyl-2-thiouridine), imG-14 (4-demethylwyosine), imG2 (isowyosine), ac⁶A (N6-acetyladenosine), inm⁵U (5-(isopentenylaminomethyl)uridine), inm⁵s²U (5-(isopentenylaminomethyl)-2-thiouridine), inm⁵Um (5-(isopentenylaminomethyl)-2′-O-methyluridine), m^{2,7}Gm (N2,7,2′-O-trimethylguanosine), m⁴₂Cm (N4,N4,2′-O-trimethylcytidine), C⁺ (agmatidine), m⁸A (8-methyladenosine), gmnm⁵s²U (geranylated 5-methylaminomethyl-2-thiouridine), gcmnm⁵s²U (geranylated 5-carboxymethylaminomethyl-2-thiouridine), or cnm⁵U (5-cyanomethyl-uridine).

In one embodiment, the modified nucleoside residues are pseudouridine or pseudouridine family residues.

In one embodiment, the modified mRNA comprises pseudouridine residues. In one embodiment, pseudouridine refers to the C-glycoside isomer of the nucleoside uridine. In one embodiment, pseudouridine residues comprise m¹acp³Ψ (1-methyl-3-(3-amino-5-carboxypropyl)pseudouridine, m¹Ψ (1-methylpseudouridine), Ψm (2′-O-methylpseudouridine, m⁵D (5-methyldihydrouridine), m³Ψ (3-methylpseudouridine), or a combination thereof. In one embodiment, said pseudouridine residues comprise 1-methylpseudouridine residues instead of uridine.

In one embodiment, the modified nucleoside residues are pseudouridine analogues. In one embodiment, a “pseudouridine analog” is any modification, variant, isoform or derivative of pseudouridine. For example, pseudouridine analogs include but are not limited to 1-carboxymethyl-pseudouridine, 1-propynyl-pseudouridine, 1-taurinomethyl-pseudouridine, 1-taurinomethyl-4-thio-pseudouridine, 1-methylpseudouridine (m¹□), 1-methyl-4-thio-pseudouridine (m¹s⁴□), 4-thio-1-methyl-pseudouridine, 3-methyl-pseudouridine (m³□), 2-thio-1-methyl-pseudouridine, 1-methyl-1-deaza-pseudouridine, 2-thio-1-methyl-1-deaza-pseudouridine, dihydropseudouridine, 2-thio-dihydropseudouridine, 2-methoxyuridine, 2-methoxy-4-thio-uridine, 4-methoxy-pseudouridine, 4-methoxy-2-thio-pseudouridine, N1-methyl-pseudouridine, 1-methyl-3-(3-amino-3-carboxypropyl)pseudouridine (acp³□), and 2′-O-methyl-pseudouridine (□m).

In some embodiments, the modified nucleobase is a modified uracil. Exemplary nucleobases and nucleosides having a modified uracil include pseudouridine (□), pyridin-4-one ribonucleoside, 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridine or 5-bromo-uridine), 3-methyl-uridine (m³U), 5-methoxy-uridine (mo⁵U), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (τcm⁵U), 1-taurinomethyl-pseudouridine, 5-taurinomethyl-2-thio-uridine (τrm⁵s²U), 1-taurinomethyl-4-thio-pseudouridine, 5-methyl-uridine (m⁵U, i.e., having the nucleobase deoxythymine), 1-methylpseudouridine (m¹□), 5-methyl-2-thio-uridine (m⁵s²U), 1-methyl-4-thio-pseudouridine (m¹s⁴□), 4-thio-1-methyl-pseudouridine, 3-methyl-pseudouridine (m3H), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine (also known as 1-methylpseudouridine (m¹□), 3-(3-amino-3-carboxypropyl)uridine (acp³U), 1-methyl-3-(3-amino-3-carboxypropyl)pseudouridine (acp³□), 5-(isopentenylaminomethyl)uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (□m), 2-thio-2′-O-methyl-uridine (s²Um), 5-methoxycarbonylmethyl-2′-O-methyl-uridine (mcm⁵Um), 5-carbamoylmethyl-2′-B-methyl-uridine (ncm⁵Um), 5-carboxymethylaminomethyl-2′-O-methyl-uridine (cmnm⁵Um), 3,2′-O-dimethyl-uridine (m³Um), 5-(isopentenylaminomethyl)-2′-B-methyl-uridine (inm⁵Um), 1-thio-uridine, deoxythymidine, 2′-F-ara-uridine, 2′-F-uridine, 2′-OH-ara-uridine, 5-(2-carbomethoxyvinyl)uridine, and 5-[3-(1-E-propenylamino)uridine.

In some embodiments, the modified nucleobase is a modified cytosine. Exemplary nucleobases and nucleosides having a modified cytosine include 5-aza-cytidine, 6-aza-cytidine, pseudoisocytidine, 3-methyl-cytidine (m³C), N4-acetyl-cytidine (ac⁴C), 5-formyl-cytidine (PC), N4-methyl-cytidine (m⁴C), 5-methyl-cytidine (m⁵C), 5-halo-cytidine (e.g., 5-iodo-cytidine), 5-hydroxymethyl-cytidine (hm⁵C), 1-methyl-pseudoisocytidine, pyrrolo-cytidine, pyrrolo-pseudoisocytidine, 2-thio-cytidine (s²C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k₂C), α-thio-cytidine, 2′-O-methyl-cytidine (Cm), 5,2′-O-dimethyl-cytidine (m⁵Cm), N4-acetyl-2′-O-methyl-cytidine (ac⁴Cm), N4,2′-O-dimethyl-cytidine (m⁴Cm), 5-formyl-2′-O-methyl-cytidine (f⁵Cm), N4,N4,2′-O-trimethyl-cytidine (m⁴₂Cm), 1-thio-cytidine, 2′-F-ara-cytidine, 2′-F-cytidine, and 2′-OH-ara-cytidine.

In some embodiments, the modified nucleobase is a modified adenine. Exemplary nucleobases and nucleosides having a modified adenine include 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m¹A), 2-methyl-adenine (m²A), N6-methyl-adenosine (m⁶A), 2-methylthio-N6-methyl-adenosine (ms² m⁶A), N6-isopentenyl-adenosine (i⁶A), 2-methylthio-N6-isopentenyl-adenosine (ms²i⁶A), N6-(cis-hydroxyisopentenyl)adenosine (io⁶A), 2-methylthio-N6-(cis-hydroxyisopentenyl)adenosine (ms²io⁶A), N6-glycinylcarbamoyl-adenosine (g⁶A), N6-threonylcarbamoyl-adenosine (t⁶A), N6-methyl-N6-threonylcarbamoyl-adenosine (m⁶t⁶A), 2-methylthio-N6-threonylcarbamoyl-adenosine (ms²g⁶A), N6,N6-dimethyl-adenosine (m62A), N6-hydroxynorvalylcarbamoyl-adenosine (hn⁶A), 2-methylthio-N6-hydroxynorvalylcarbamoyl-adenosine (ms² hn⁶A), N6-acetyl-adenosine (ac⁶A), 7-methyl-adenine, 2-methylthio-adenine, 2-methoxy-adenine, α-thio-adenosine, 2′-O-methyl-adenosine (Am), N6,2′-O-dimethyl-adenosine (m⁶Am), N6,N6,2′-O-trimethyl-adenosine (m⁶₂Am), 1,2′-O-dimethyl-adenosine (m¹Am), 2′-p-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the modified nucleobase is a modified guanine. Exemplary nucleobases and nucleosides having a modified guanine include inosine (I), 1-methyl-inosine (m¹₁), wyosine (imG), methylwyosine (mimG), 4-demethyl-wyosine (imG-14), isowyosine (imG2), wybutosine (yW), peroxywybutosine (o₂yW), hydroxywybutosine (OHyW), undermodified hydroxywybutosine (OHyW*), 7-deaza-guanosine, queuosine (Q), epoxyqueuosine (oQ), galactosyl-queuosine (galQ), mannosyl-queuosine (manQ), 7-cyano-7-deaza-guanosine (preQ₀), 7-aminomethyl-7-deaza-guanosine (preQ₁), archaeosine (G), 7-deaza-8-aza-guanosine, 6-thio-guanosine, 6-thio-7-deaza-guanosine, 6-thio-7-deaza-8-aza-guanosine, 7-methyl-guanosine (m⁷G), 6-thio-7-methyl-guanosine, 7-methyl-inosine, 6-methoxy-guanosine, 1-methyl-guanosine (m¹G), N2-methyl-guanosine (m²G), N2,N2-dimethyl-guanosine (m²₂G), N2,7-dimethyl-guanosine (m²,7G), N2,N2,7-dimethyl-guanosine (m²,2,7G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m²Gm), N2,N2-dimethyl-2′-O-methyl-guanosine (m²²Gm), 1-methyl-2′-O-methyl-guanosine (m¹Gm), N2,7-dimethyl-2′-O-methyl-guanosine (m²′7Gm), 2′-O-methyl-inosine (Im), 1,2′-O-dimethyl-inosine (m¹Im), and 2′-O-ribosylguanosine (phosphate) (Gr(p)).

The nucleobase of the nucleotide can be independently selected from a purine, a pyrimidine, a purine or pyrimidine analog. For example, the nucleobase can each be independently selected from adenine, cytosine, guanine, uracil, or hypoxanthine. In another embodiment, the nucleobase can also include, for example, naturally-occurring and synthetic derivatives of a base, including pyrazolo[3,4-d]pyrimidines, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-propynyl uracil and cytosine, 6-azo uracil, cytosine and thymine, 5-uracil (pseudouracil), 4-thiouracil, 8-halo (e.g., 8-bromo), 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, deazaguanine, 7-deazaguanine, 3-deazaguanine, deazaadenine, 7-deazaadenine, 3-deazaadenine, pyrazolo[3,4-d]pyrimidine, imidazo[1,5-a]1,3,5 triazinones, 9-deazapurines, imidazo[4,5-d]pyrazines, thiazolo[4,5-d]pyrimidines, pyrazin-2-ones, 1,2,4-triazine, pyridazine; and 1,3,5 triazine. When the nucleotides are depicted using the shorthand A, G, C, T or U, each letter refers to the representative base and/or derivatives thereof, e.g., A includes adenine or adenine analogs, e.g., 7-deaza adenine).

### Modifications on the Internucleoside Linkage

The modified nucleotides, which may be incorporated into a polynucleotide, primary construct, or mRNA molecule, can be modified on the internucleoside linkage (e.g., phosphate backbone). Herein, in the context of the polynucleotide backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage as described herein. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylene-phosphonates).

The α-thio substituted phosphate moiety is provided to confer stability to RNA and DNA polymers through the unnatural phosphorothioate backbone linkages. Phosphorothioate DNA and RNA have increased nuclease resistance and subsequently a longer half-life in a cellular environment. Phosphorothioate linked polynucleotides, primary constructs, or mmRNA molecules are expected to also reduce the innate immune response through weaker binding/activation of cellular innate immune molecules.

In specific embodiments, a modified nucleoside includes an alpha-thio-nucleoside (e.g., 5′-O-(1-thiophosphate)-adenosine, 5′-O-(1-thiophosphate)-cytidine (α-thio-cytidine), 5′-O-(1-thiophosphate)-guanosine, 5′-O-(1-thiophosphate)-uridine, or 5′-O-(1-thiophosphate)-pseudouridine).

Other internucleoside linkages that may be employed according to the present invention, including internucleoside linkages which do not contain a phosphorous atom, are described herein below.

### Combinations of Modified Sugars, Nucleobases, and Internucleoside Linkages

The polynucleotides, primary constructs, and mmRNA of the invention can include a combination of modifications to the sugar, the nucleobase, and/or the internucleoside linkage.

In another embodiment, the purified preparation of RNA, oligoribonucleotide, or polyribonucleotide of the methods and compositions of the present invention comprises a combination of two or more of the above-described modifications. In another embodiment, the purified preparation of the RNA or oligoribonucleotide comprises a combination of three or more of the above-described modifications. In another embodiment, the purified preparation of the RNA or oligoribonucleotide comprises a combination of more than three of the above-described modifications.

In one embodiment, the modified mRNAs comprise in vitro-synthesized modified mRNAs.

In one embodiment, the present invention comprises one or more modified mRNAs encoding an HSV glycoprotein. In one embodiment, the modified RNA comprises pseudouridine or pseudouridine family residues. In another embodiment, the modified mRNAs of the present invention are capable of directing protein expression of HSV glycoproteins encoded thereon.

In another embodiment, the present invention provides an in vitro-transcribed mRNA molecule encoding an HSV glycoprotein, comprising a pseudouridine. In another embodiment, the present invention provides a synthetic mRNA molecule encoding an HSV glycoprotein, comprising a pseudouridine.

In another embodiment, an in vitro-transcribed mRNA molecule of the methods and compositions of the present invention is synthesized by T7 phage RNA polymerase. In another embodiment, the molecule is synthesized by SP6 phage RNA polymerase. In another embodiment, the molecule is synthesized by T3 phage RNA polymerase. In another embodiment, the molecule is synthesized by a polymerase selected from the above polymerases. In another embodiment, the mRNA is synthesized chemically on a column similar to DNA.

In another embodiment, the nucleoside that is modified in an RNA, oligoribonucleotide, or polyribonucleotide of the methods and compositions of the present invention is uridine (U). In another embodiment, the modified nucleoside is cytidine (C). In another embodiment, the modified nucleoside is adenine (A). In another embodiment the modified nucleoside is guanine (G).

In another embodiment, the modified mRNA of the methods and compositions of the present invention further comprises a poly-A tail. In another embodiment, the modified mRNA of the methods and compositions of the present invention does not comprise a poly-A tail. Each possibility represents a separate embodiment of the present invention.

In another embodiment, the modified mRNA of the methods and compositions of the present invention comprises an m7GpppG cap. In another embodiment, the modified mRNA of the methods and compositions of the present invention does not comprise an m7GpppG cap. In another embodiment, the modified mRNA of the methods and compositions of the present invention comprises a 3′-O-methyl-m7GpppG. In another embodiment, the modified mRNA of methods and composition of the present invention comprise a non-reversible cap analog, which, in one embodiment, is added during transcription of the mRNA. In another embodiment, the modified mRNA of methods and composition of the present invention comprise an anti-reverse cap analog. Each possibility represents a separate embodiment of the present invention.

In another embodiment, the modified mRNA of the methods and compositions of the present invention further comprises a cap-independent translational enhancer. In another embodiment, the modified mRNA of the methods and compositions of the present invention does not comprise a cap-independent translational enhancer. In another embodiment, the cap-independent translational enhancer is a tobacco etch virus (TEV) cap-independent translational enhancer. In another embodiment, the cap-independent translational enhancer is any other cap-independent translational enhancer known in the art. Each possibility represents a separate embodiment of the present invention.

In one embodiment, “pseudouridine” refers to m¹acp³Ψ (1-methyl-3-(3-amino-5-carboxypropyl)pseudouridine. In another embodiment, the term refers to m¹Ψ (1-methylpseudouridine). In another embodiment, the term refers to Ψm (2′-O-methylpseudouridine. In another embodiment, the term refers to m⁵D (5-methyldihydrouridine). In another embodiment, the term refers to m³Ψ (3-methylpseudouridine). In another embodiment, the modified nucleoside is 4′ (pseudouridine). In another embodiment, the term refers to a pseudouridine moiety that is not further modified. In another embodiment, the term refers to a monophosphate, diphosphate, or triphosphate of any of the above pseudouridines. In another embodiment, the term refers to any other pseudouridine known in the art. Each possibility represents a separate embodiment of the present invention.

In another embodiment, the modified RNA comprises a modified nucleoside, which in one embodiment, comprises m⁵C, m5U, m⁶A, s²U,□□, 2′-O-methyl-U, 2′-O-methylpseudouridine, or a combination thereof.

In another embodiment, the present invention provides a method for delivering a recombinant protein to a subject, the method comprising the step of contacting the subject with a modified mRNA of the methods and compositions of the present invention, thereby delivering a recombinant protein to a subject.

In another embodiment, a method of the present invention comprises increasing the number, percentage, or frequency of modified uridine nucleosides in the RNA molecule to decrease immunogenicity or increase efficiency of translation. In one embodiment, the number of modified uridine residues in an RNA, oligoribonucleotide, or polyribonucleotide molecule determines the magnitude of the effects observed in the present invention.

In another embodiment, between 0.1% and 100% of the uridine residues in the modified mRNAs of the methods and compositions of the present invention are modified (e.g. by the presence of pseudouridine). In another embodiment, 0.1% of the residues are modified. In another embodiment, 0.2%. In another embodiment, the fraction is 0.3%. In another embodiment, the fraction is 0.4%. In another embodiment, the fraction is 0.5%. In another embodiment, the fraction is 0.6%. In another embodiment, the fraction is 0.8%. In another embodiment, the fraction is 1%. In another embodiment, the fraction is 1.5%. In another embodiment, the fraction is 2%. In another embodiment, the fraction is 2.5%. In another embodiment, the fraction is 3%. In another embodiment, the fraction is 4%. In another embodiment, the fraction is 5%. In another embodiment, the fraction is 6%. In another embodiment, the fraction is 8%. In another embodiment, the fraction is 10%. In another embodiment, the fraction is 12%. In another embodiment, the fraction is 14%. In another embodiment, the fraction is 16%. In another embodiment, the fraction is 18%. In another embodiment, the fraction is 20%. In another embodiment, the fraction is 25%. In another embodiment, the fraction is 30%. In another embodiment, the fraction is 35%. In another embodiment, the fraction is 40%. In another embodiment, the fraction is 45%. In another embodiment, the fraction is 50%. In another embodiment, the fraction is 60%. In another embodiment, the fraction is 70%. In another embodiment, the fraction is 80%. In another embodiment, the fraction is 90%. In another embodiment, the fraction is 100%.

In another embodiment, the fraction is less than 5%. In another embodiment, the fraction is less than 3%. In another embodiment, the fraction is less than 1%. In another embodiment, the fraction is less than 2%. In another embodiment, the fraction is less than 4%. In another embodiment, the fraction is less than 6%. In another embodiment, the fraction is less than 8%. In another embodiment, the fraction is less than 10%. In another embodiment, the fraction is less than 12%. In another embodiment, the fraction is less than 15%. In another embodiment, the fraction is less than 20%. In another embodiment, the fraction is less than 30%. In another embodiment, the fraction is less than 40%. In another embodiment, the fraction is less than 50%. In another embodiment, the fraction is less than 60%. In another embodiment, the fraction is less than 70%

In another embodiment, 0.1% of the residues of a given uridine nucleotide are modified. In another embodiment, the fraction of the nucleotide is 0.2%. In another embodiment, the fraction is 0.3%. In another embodiment, the fraction is 0.4%. In another embodiment, the fraction is 0.5%. In another embodiment, the fraction is 0.6%. In another embodiment, the fraction is 0.8%. In another embodiment, the fraction is 1%. In another embodiment, the fraction is 1.5%. In another embodiment, the fraction is 2%. In another embodiment, the fraction is 2.5%. In another embodiment, the fraction is 3%. In another embodiment, the fraction is 4%. In another embodiment, the fraction is 5%. In another embodiment, the fraction is 6%. In another embodiment, the fraction is 8%. In another embodiment, the fraction is 10%. In another embodiment, the fraction is 12%. In another embodiment, the fraction is 14%. In another embodiment, the fraction is 16%. In another embodiment, the fraction is 18%. In another embodiment, the fraction is 20%. In another embodiment, the fraction is 25%. In another embodiment, the fraction is 30%. In another embodiment, the fraction is 35%. In another embodiment, the fraction is 40%. In another embodiment, the fraction is 45%. In another embodiment, the fraction is 50%. In another embodiment, the fraction is 60%. In another embodiment, the fraction is 70%. In another embodiment, the fraction is 80%. In another embodiment, the fraction is 90%. In another embodiment, the fraction is 100%.

In another embodiment, the fraction of the given uridine nucleotide is less than 8%. In another embodiment, the fraction is less than 10%. In another embodiment, the fraction is less than 5%. In another embodiment, the fraction is less than 3%. In another embodiment, the fraction is less than 1%. In another embodiment, the fraction is less than 2%. In another embodiment, the fraction is less than 4%. In another embodiment, the fraction is less than 6%. In another embodiment, the fraction is less than 12%. In another embodiment, the fraction is less than 15%. In another embodiment, the fraction is less than 20%. In another embodiment, the fraction is less than 30%. In another embodiment, the fraction is less than 40%. In another embodiment, the fraction is less than 50%. In another embodiment, the fraction is less than 60%. In another embodiment, the fraction is less than 70%.

In another embodiment, the terms “ribonucleotide,” “oligoribonucleotide,” and polyribonucleotide refers to, in one embodiment, compounds comprising nucleotides in which the sugar moiety is ribose. In another embodiment, the term includes both RNA and RNA derivates in which the backbone is modified. Numerous RNA backbone modifications are known in the art and contemplated in the present invention. In one embodiment, modified RNA is a PNA (peptide nucleic acid). PNA contain peptide backbones and nucleotide bases and are able to bind, in another embodiment, to both DNA and RNA molecules. In another embodiment, the nucleotide is modified by replacement of one or more phosphodiester bonds with a phosphorothioate bond. In another embodiment, the artificial nucleic acid contains any other variant of the phosphate backbone of native nucleic acids known in the art. Each nucleic acid derivative represents a separate embodiment of the present invention.

Methods for production of nucleic acids having modified backbones are well known in the art, and are described, for example in U.S. Pat. Nos. 5,723,335 and 5,663,153 issued to Hutcherson et al. and related PCT publication WO95/26204. Each method represents a separate embodiment of the present invention.

The nucleic acid of interest can be purified by any method known in the art, or any method to be developed, so long as the method of purification removes contaminants from the nucleic acid preparation and thereby substantially reduces the immunogenicity potential of the nucleic acid preparation. In one embodiment, the nucleic acid of interest is purified using high-performance liquid chromatography (HPLC). In another embodiment, the nucleic acid of interest is purified by contacting the nucleic acid of interest with the bacterial enzyme RNase III. In other various embodiments, any method of nucleic acid purification that substantially reduces the immunogenicity of the nucleic acid preparation can be used. Non-limiting examples of purification methods that can be used with the compositions and methods of the invention liquid chromatography separation and enzyme digestion, each used alone or in any combination, simultaneously or in any order. Non-limiting examples of liquid chromatography separation include HPLC and fast protein liquid chromatography (FPLC). Materials useful in the HPLC and FPLC methods of the invention include, but are not limited to, cross-linked polystyrene/divinylbenzene (PS/DVB), PS/DVB-C18, PS/DVB-alkylated, Helix DNA columns (Varian), Eclipse dsDNA Analysis Columns (Agilent Technologies), Reverse-phase 5 (RPC-5) exchange material, DNAPac, ProSwift, and bio-inert UltiMate® 3000 Titanium columns (Dionex). Enzymes useful in the enzyme digestion methods of the invention include any enzyme able to digest any contaminant in a nucleic acid preparation of the invention, such as, for example a dsRNA contaminant, and include but are not limited to, RNase III, RNase V1, Dicer, and Chipper (see Fruscoloni et al., 2002, PNAS 100:1639) Non-limiting examples of assays for assessing the purity of the nucleic acid of interest include a dot-blot assay, a Northern blot assay, and a dendritic cell activation assay, as described elsewhere herein.

In another embodiment, the modified mRNA of the methods and compositions of the present invention is significantly less immunogenic than an unmodified in vitro-synthesized mRNA molecule with the same sequence. In another embodiment, the modified mRNA molecule is 2-fold less immunogenic than its unmodified counterpart. In another embodiment, immunogenicity is reduced by a 3-fold factor. In another embodiment, immunogenicity is reduced by a 5-fold factor. In another embodiment, immunogenicity is reduced by a 7-fold factor. In another embodiment, immunogenicity is reduced by a 10-fold factor. In another embodiment, immunogenicity is reduced by a 15-fold factor. In another embodiment, immunogenicity is reduced by a fold factor. In another embodiment, immunogenicity is reduced by a 50-fold factor. In another embodiment, immunogenicity is reduced by a 100-fold factor. In another embodiment, immunogenicity is reduced by a 200-fold factor. In another embodiment, immunogenicity is reduced by a 500-fold factor. In another embodiment, immunogenicity is reduced by a 1000-fold factor. In another embodiment, immunogenicity is reduced by a 2000-fold factor. In another embodiment, immunogenicity is reduced by another fold difference.

In another embodiment, “significantly less immunogenic” refers to a detectable decrease in immunogenicity. In another embodiment, the term refers to a fold decrease in immunogenicity (e.g. 1 of the fold decreases enumerated above). In another embodiment, the term refers to a decrease such that an effective amount of the modified mRNA can be administered without triggering a detectable immune response. In another embodiment, the term refers to a decrease such that the modified mRNA can be repeatedly administered without eliciting an immune response sufficient to detectably reduce expression of the recombinant protein. In another embodiment, the decrease is such that the modified mRNA can be repeatedly administered without eliciting an immune response sufficient to eliminate detectable expression of the recombinant protein.

Methods of determining immunogenicity are well known in the art, and described in detail in U.S. Pat. No. 8,278,036 which is hereby incorporated by reference herein.

In another embodiment, the modified mRNA of the methods and compositions of the present invention is translated in the cell more efficiently than an unmodified mRNA molecule with the same sequence. In another embodiment, the modified mRNA exhibits enhanced ability to be translated by a target cell. In another embodiment, translation is enhanced by a factor of 2-fold relative to its unmodified counterpart. In another embodiment, translation is enhanced by a 3-fold factor. In another embodiment, translation is enhanced by a 5-fold factor. In another embodiment, translation is enhanced by a 7-fold factor. In another embodiment, translation is enhanced by a 10-fold factor. In another embodiment, translation is enhanced by a 15-fold factor. In another embodiment, translation is enhanced by a 20-fold factor. In another embodiment, translation is enhanced by a 50-fold factor. In another embodiment, translation is enhanced by a 100-fold factor. In another embodiment, translation is enhanced by a 200-fold factor. In another embodiment, translation is enhanced by a 500-fold factor. In another embodiment, translation is enhanced by a 1000-fold factor. In another embodiment, translation is enhanced by a 2000-fold factor. In another embodiment, the factor is 10-1000-fold. In another embodiment, the factor is 10-100-fold. In another embodiment, the factor is 10-200-fold. In another embodiment, the factor is 10-300-fold. In another embodiment, the factor is 10-500-fold. In another embodiment, the factor is 20-1000-fold. In another embodiment, the factor is 30-1000-fold. In another embodiment, the factor is 50-1000-fold. In another embodiment, the factor is 100-1000-fold. In another embodiment, the factor is 200-1000-fold. In another embodiment, translation is enhanced by any other significant amount or range of amounts. Each possibility represents a separate embodiment of the present invention.

Methods of determining translation efficiency are well known in the art, and include, e.g. measuring the activity of an encoded reporter protein (e.g luciferase or renilla or green fluorescent protein [Wall A A, Phillips A M et al, Effective translation of the second cistron in two Drosophila dicistronic transcripts is determined by the absence of in-frame AUG codons in the first cistron. J Biol Chem 2005; 280(30): 27670-8]), or measuring radioactive label incorporated into the translated protein (Ngosuwan J, Wang N M et al, Roles of cytosolic Hsp70 and Hsp40 molecular chaperones in post-translational translocation of pre-secretory proteins into the endoplasmic reticulum. J Biol Chem 2003; 278(9): 7034-42). Each method represents a separate embodiment of the present invention.

In another embodiment, the target cell of the method of the present invention is a dendritic cell. In another embodiment, the target cell of the method of the present invention is a macrophage. In another embodiment, the target cell of the method of the present invention is a B cell. In another embodiment, the target cell of the method of the present invention is another antigen presenting cell. In another embodiment, the target cell of methods of the present invention is a mucosal cell. In another embodiment, the target cell of methods of the present invention is an epithelial cell. In another embodiment, the cell is a skin cell. In another embodiment, the cell is an epidermal cell. In another embodiment, the cell is a keratinocyte. In another embodiment, the cell is a Merkel cell, melanocyte or Langerhans cell. Each possibility represents a separate embodiment of the present invention.

### Methods of Treatment and Uses of the Compositions

The present invention also provides methods of vaccinating a subject against HSV and treating, impeding, inhibiting, reducing the incidence of, or suppressing an HSV infection or a symptom or manifestation thereof, comprising administration of a composition of the present invention.

In one embodiment, the present invention provides a method for treating an HSV infection in a subject, comprising contacting said subject with a composition comprising one or more modified mRNAs, wherein each of said modified mRNAs encodes an HSV glycoprotein or immunogenic fragment thereof.

In another embodiment, the present invention provides a method for suppressing an HSV infection in a subject, comprising contacting said subject with a composition comprising one or more modified mRNAs, wherein each of said modified mRNAs encodes an HSV glycoprotein or immunogenic fragment thereof.

In another embodiment, the present invention provides a method for inhibiting an HSV infection in a subject, comprising contacting said subject with a composition comprising one or more modified mRNAs, wherein each of said modified mRNAs encodes an HSV glycoprotein or immunogenic fragment thereof.

In another embodiment, the present invention provides a method for reducing the incidence of HSV infection in a subject, comprising contacting said subject with a composition comprising one or more modified mRNAs, wherein each of said modified mRNAs encodes an HSV glycoprotein or immunogenic fragment thereof.

In one embodiment, the HSV infection is an HSV-1 infection. In another embodiment, the HSV infection is an HSV-2 infection.

In one embodiment, the subject is administered HSV-1 glycoproteins for methods of treating, inhibiting, suppressing, etc. an HSV-1 infection. In another embodiment, the subject is administered HSV-2 glycoproteins for methods of treating, inhibiting, suppressing, etc. an HSV-2 infection. In another embodiment, the subject is administered HSV-1 glycoproteins for methods of treating, inhibiting, suppressing, etc. an HSV-1 infection, HSV-2 infection, or a combination thereof. In another embodiment, the subject is administered HSV-2 glycoproteins for methods of treating, inhibiting, suppressing, etc. an HSV-1 infection, HSV-2 infection, or a combination thereof. In one embodiment, administration of HSV-1 glycoproteins (e.g., gC1, gD1, gE1, or a combination thereof) treats or prevents HSV-1 and HSV-2 infection. In another embodiment, administration of HSV-2 glycoproteins (e.g., gC2, gD2 and gE2, or a combination thereof) treats or prevents HSV-1 and HSV-2 infection.

According to this aspect and in one embodiment, the present invention provides a method for treating, suppressing, inhibiting, or reducing the incidence of Herpes Simplex Virus 1 (HSV-1) infection in a subject, comprising contacting said subject with a composition comprising one or more modified mRNAs, wherein each of said modified mRNAs encodes an HSV-1 glycoprotein or immunogenic fragment thereof.

In one embodiment, the present invention provides a method for treating, suppressing, inhibiting, or reducing the incidence of Herpes Simplex Virus 2 (HSV-2) infection in a subject, comprising contacting said subject with a composition comprising one or more modified mRNAs, wherein each of said modified mRNAs encodes an HSV-2 glycoprotein or immunogenic fragment thereof.

In one embodiment, said contacting is via administration to said subject.

In another embodiment, the present invention provides a method of treating, suppressing, inhibiting, or reducing the incidence of an HSV infection in a subject, the method comprising the step of administering to said subject an immunogenic composition comprising modified mRNAs encoding: (a) an HSV gD or immunogenic fragment thereof, (b) an HSV gC or fragment thereof as described herein; (c) an HSV gE or fragment thereof as described herein, or a combination thereof.

In another embodiment, the present invention provides a method of treating, suppressing, inhibiting, or reducing the incidence of an HSV infection in a subject, the method comprising the step of administering to said subject an immunogenic composition comprising modified mRNAs encoding: (a) an HSV-2 gD or immunogenic fragment thereof, (b) an HSV-2 gC or fragment thereof as described herein; and (c) an HSV-2 gE or fragment thereof as described herein, or a combination thereof.

In another embodiment, the present invention provides a method of treating, suppressing, inhibiting, or reducing the incidence of an HSV infection in a subject, the method comprising the step of administering to said subject an immunogenic composition comprising modified mRNAs encoding: (a) an HSV-1 gD or immunogenic fragment thereof, (b) an HSV-1 gC or fragment thereof as described herein; and (c) an HSV-1 gE or fragment thereof as described herein, or a combination thereof.

In another embodiment, the present invention provides a method of inducing an anti-HSV immune response in a subject, the method comprising the step of administering to said subject an immunogenic composition comprising modified mRNAs encoding: (a) an HSV gD or immunogenic fragment thereof, (b) an HSV gC or fragment thereof as described herein; (c) an HSV gE or fragment thereof as described herein, or a combination thereof.

In another embodiment, the present invention provides a method of inducing an anti-HSV immune response in a subject, the method comprising the step of administering to said subject an immunogenic composition comprising modified mRNAs encoding: (a) an HSV-2 gD or immunogenic fragment thereof, (b) an HSV-2 gC or fragment thereof as described herein; and (c) an HSV-2 gE or fragment thereof as described herein, or a combination thereof.

In another embodiment, the present invention provides a method of inducing an anti-HSV immune response in a subject, the method comprising the step of administering to said subject an immunogenic composition comprising modified mRNAs encoding: (a) an HSV-1 gD or immunogenic fragment thereof; (b) an HSV-1 gC or fragment thereof as described herein; and (c) an HSV-1 gE or fragment thereof as described herein, or a combination thereof.

In another embodiment, the present invention provides a method of inhibiting a primary HSV infection in a subject, the method comprising the step of administering to the subject a composition of the present invention. In another embodiment, the present invention provides a method of treating an HSV infection in a subject, the method comprising the step of administering to said subject a composition of the present invention. In another embodiment, the present invention provides a method of reducing the incidence of an HSV infection in a subject, the method comprising the step of administering to said subject a composition of the present invention. In another embodiment, the present invention provides a method of inhibiting a flare following a primary HSV infection in a subject, the method comprising the step of administering to said subject a composition of the present invention.

In one embodiment, the present invention provides methods of treating and/or suppressing a primary HSV infection and/or a secondary HSV infection. In one embodiment, a “primary” infection refers to a first-time infection. In one embodiment, a “secondary” infection refers to a recurrence of an HSV infection.

In one embodiment, a “flare” or “recurrence” refers to reinfection of skin tissue following latent neuronal HSV infection. In another embodiment, the terms refer to reactivation of HSV after a latency period. In another embodiment, the terms refer to symptomatic HSV lesions following a non-symptomatic latency period.

In another embodiment, the present invention provides a method of inhibiting spread of HSV. In one embodiment, the spread from DRG to skin is inhibited. In one embodiment, cell-to-cell spread of HSV is inhibited. In one embodiment, anterograde spread is inhibited. In one embodiment, retrograde spread is inhibited. “DRG” refers, in one embodiment, to a neuronal cell body and in another embodiment, contain the neuron cell bodies of nerve fibers. In another embodiment, the term refers to any other definition of “DRG” used in the art. In another embodiment, spread of HSV to neural tissue is inhibited.

In another embodiment, the present invention provides a method of inhibiting a recurrence following a primary HSV infection in a subject, the method comprising the step of administering to said subject a composition of the present invention. In another embodiment, the present invention provides a method of preventing a recurrence following a primary HSV infection in a subject, the method comprising the step of administering to said subject a composition of the present invention.

In another embodiment, the present invention provides a method of inhibiting an HSV labialis following a primary HSV infection in a subject, the method comprising the step of administering to said subject a composition of the present invention.

In another embodiment, the present invention provides a method of preventing a recurrence of an HSV infection, the method comprising the step of administering to said subject a composition of the present invention. In another embodiment, the present invention provides a method of diminishing the severity of a recurrence of an HSV infection, the method comprising the step of administering to said subject a composition of the present invention. In another embodiment, the present invention provides a method of reducing the frequency of a recurrence of an HSV infection, the method comprising the step of administering to said subject a composition of the present invention. In one embodiment, the present invention provides any of the described methods in an HIV-infected subject.

In another embodiment, the present invention provides a method of treating an HSV encephalitis in a subject, the method comprising the step of administering to said subject a composition of the present invention. In another embodiment, the present invention provides a method of reducing the incidence of an HSV encephalitis in a subject, the method comprising the step of administering to said subject a composition of the present invention. “HSV encephalitis” refers, in one embodiment, to an encephalitis caused by a Herpes Simplex Virus-1 (HSV). In another embodiment, the term refers to an encephalitis associated with HSV. In another embodiment, the term refers to any other type of HSV-mediated encephalitis known in the art.

In another embodiment, the present invention provides a method of treating or reducing an HSV neonatal infection in a subject, the method comprising the step of administering to said subject a composition of the present invention.

In another embodiment, the present invention provides a method for introducing an HSV glycoprotein to a cell of a subject, comprising contacting said cell with an in vitro-transcribed mRNA molecule encoding the recombinant protein, wherein said in vitro-transcribed mRNA molecule further comprises a modified nucleoside, thereby introducing said HSV glycoprotein into said cell of said subject.

In another embodiment, the present invention provides a method for inducing a mammalian cell to produce an HSV glycoprotein, comprising contacting said mammalian cell with an in vitro-synthesized mRNA molecule encoding the HSV glycoprotein, the in vitro-synthesized mRNA molecule comprising a pseudouridine, thereby inducing said mammalian cell to produce said HSV glycoprotein.

It is to be understood that reference to HSV herein refers in one embodiment, to HSV-1, while in another embodiment, to HSV-2, while in another embodiment, to HSV-1 and HSV-2.

“HSV-1” refers, in another embodiment, to a Herpes Simplex Virus-1. In another embodiment, the term refers to a KOS strain. In another embodiment, the term refers to an F strain. In another embodiment, the term refers to an NS strain. In another embodiment, the term refers to a CL101 strain. In another embodiment, the term refers to a “17” strain. In another embodiment, the term refers to a “17+syn” strain. In another embodiment, the term refers to a MacIntyre strain. In another embodiment, the term refers to an MP strain. In another embodiment, the term refers to an HF strain. In another embodiment, the term refers to any other HSV-1 strain known in the art.

“HSV-2” refers, in another embodiment, to a Herpes Simplex Virus-2. In another embodiment, the term refers to an HSV-2 333 strain. In another embodiment, the term refers to a 2.12 strain. In another embodiment, the term refers to an HG52 strain. In another embodiment, the term refers to an MS strain. In another embodiment, the term refers to a G strain. In another embodiment, the term refers to a 186 strain. In another embodiment, the term refers to any other HSV-2 strain known in the art.

In another embodiment, the present invention provides a method of vaccinating a subject against an HSV infection, the method comprising the step of administering to said subject a composition of the present invention. In another embodiment, the present invention provides a method of suppressing an HSV infection in a subject, the method comprising the step of administering to said subject a composition of the present invention. In another embodiment, the present invention provides a method of impeding an HSV infection in a subject, the method comprising the step of administering to said subject a composition of the present invention. In another embodiment, the present invention provides a method of impeding a primary HSV infection in a subject, the method comprising the step of administering to said subject a composition of the present invention. In another embodiment, the present invention provides a method of impeding neuronal HSV spread in a subject, the method comprising the step of administering to said subject a composition of the present invention.

The terms “impeding an HSV infection” and “impeding a primary HSV infection” refer, in another embodiment, to decreasing the titer of infectious virus. In another embodiment, the terms refer to decreasing the extent of viral replication.

In another embodiment, the present invention provides a method of reducing the incidence of an HSV-mediated herpetic ocular disease in a subject, the method comprising the step of administering to said subject a composition of the present invention. In another embodiment, the present invention provides a method of treating an HSV-1 corneal infection or herpes keratitis in a subject, the method comprising the step of administering to said subject a composition of the present invention. In another embodiment, the present invention provides a method of reducing the incidence of an HSV-1 corneal infection or herpes keratitis in a subject, the method comprising the step of administering to said subject a composition of the present invention.

In another embodiment, the present invention provides a method of treating, suppressing or inhibiting an HSV genital infection, the method comprising the step of administering to said subject a composition of the present invention. In another embodiment, the present invention provides a method of treating, suppressing or inhibiting any manifestation of recurrent HSV infection, the method comprising the step of administering to said subject a composition of the present invention.

In another embodiment, the present invention provides a method of reducing the incidence of an HSV-mediated genital ulcer disease in a subject, the method comprising the step of administering to said subject a composition of the present invention. In another embodiment, the present invention provides a method of impeding an establishment of a latent HSV infection in a subject, the method comprising the step of administering to said subject a composition of the present invention.

In one embodiment, the present invention provides a method of treating, suppressing or inhibiting a genital herpes infection in a subject, comprising the step of administering to said subject a composition of the present invention. In another embodiment, the present invention provides a method of treating, suppressing or inhibiting an oral herpes infection in a subject, comprising the step of administering to said subject a composition of the present invention.

In another embodiment, the present invention provides a method of reducing the incidence of an HSV-mediated encephalitis in a subject, the method comprising the step of administering to said subject a composition of the present invention.

In another embodiment, the herpes-mediated encephalitis treated or prevented by a method of the present invention is a focal herpes encephalitis. In another embodiment, the herpes-mediated encephalitis is a neonatal herpes encephalitis. In another embodiment, the herpes-mediated encephalitis is any other type of herpes-mediated encephalitis known in the art.

In another embodiment, the present invention provides a method of treating or reducing the incidence of a disease, disorder, or symptom associated with or secondary to an HSV-mediated encephalitis in a subject, the method comprising the step of administering to said subject a composition of the present invention.

In another embodiment, the present invention provides a method of treating, reducing the pathogenesis of, ameliorating the symptoms of, ameliorating the secondary symptoms of, reducing the incidence of, prolonging the latency to a relapse of an HSV infection in a subject, comprising the step of administering to the subject a composition of the present invention.

In another embodiment, the present invention provides a method of protecting a subject against formation of a zosteriform lesion or an analogous outbreak in a human subject. In another embodiment, the present invention provides a method of inhibiting the formation of an HSV zosteriform lesion or an analogous outbreak in a human subject.

“Zosteriform” refers, in one embodiment, to skin lesions characteristic of an HSV infection, particularly during reactivation infection, which, in one embodiment, begin as a rash and follow a distribution near dermatomes, commonly occurring in a strip or belt-like pattern. In one embodiment, the rash evolves into vesicles or small blisters filled with serous fluid. In one embodiment, zosteriform lesions form in mice as a result of contact with HSV. In another embodiment, zosteriform lesions form in humans as a result of contact with HSV. “Zosteriform spread” refers, in one embodiment, to an HSV infection that spreads from the ganglia to secondary skin sites within the dermatome. In another embodiment, the term refers to spread within the same dermatome as the initial site of infection. In another embodiment, the term refers to any other definition of “zosteriform spread” known in the art. “Outbreak”, in another embodiment, refers to a sudden increase in symptoms of a disease or in the spread or prevalence of a disease, and in one embodiment, refers to a sudden increase in zosteriform lesions, while in another embodiment, “outbreak” refers to a sudden eruption of zosteriform lesions.

In one embodiment, the present invention provides a method of impeding the formation of a dermatome lesion or an analogous condition in a subject. In one embodiment, dermatome lesions form as a result of contact with HSV. In another embodiment, dermatome lesions most often develop when the virus reactivates from latency in the ganglia and in one embodiment, spreads down nerves, in one embodiment, causing a recurrent infection.

It is to be understood that the methods of the present invention may be used to treat, inhibit, suppress, etc. an HSV infection or primary or secondary symptoms related to such an infection following exposure of the subject to HSV. In another embodiment, the subject has been infected with HSV before vaccination. In another embodiment, the subject is at risk for HSV infection. In another embodiment, whether or not the subject has been infected with HSV at the time of vaccination, vaccination by a method of the present invention is efficacious in treating, inhibiting, suppressing, etc. an HSV infection or primary or secondary symptoms related to such an infection.

In one embodiment, “treating” refers to either therapeutic treatment or prophylactic or preventative measures, wherein the object is to prevent or lessen the targeted pathologic condition or disorder as described hereinabove. Thus, in one embodiment, treating may include directly affecting or curing, suppressing, inhibiting, preventing, reducing the severity of, delaying the onset of, reducing symptoms associated with the disease, disorder or condition, or a combination thereof. Thus, in one embodiment, “treating” refers inter alia to delaying progression, expediting remission, inducing remission, augmenting remission, speeding recovery, increasing efficacy of or decreasing resistance to alternative therapeutics, or a combination thereof. In one embodiment, “preventing” refers, inter alia, to delaying the onset of symptoms, preventing relapse to a disease, decreasing the number or frequency of relapse episodes, increasing latency between symptomatic episodes, or a combination thereof. In one embodiment, “suppressing” or “inhibiting”, refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

In one embodiment, the compositions and methods of the present invention are effective in lowering HSV acquisition rates, duration of HSV infection, frequency of HSV reactivation, or a combination thereof. In another embodiment, the compositions and methods of the present invention are effective in treating or inhibiting genital ulcer disease, which in one embodiment, entails decreasing the severity or frequency of HSV genital ulcer disease. In one embodiment, the compositions and methods of the present invention block immune evasion from complement. In one embodiment, vaccination with mRNA-encoded HSV subunits may produce high titers of neutralizing antibodies or potent T-cell responses; however, upon subsequent infection, HSV immune evasion molecules may block the activities of antibodies or T cells, thereby reducing composition efficacy. In one embodiment, the compositions and methods of the present invention incorporate strategies to block virus mediated immune evasion by, in one embodiment, enhancing the effectiveness of e.g. a gD-1 subunit composition using gC-1 to prevent immune evasion from complement.

In one embodiment, studies in guinea pigs and mice suggest that viral load in ganglia correlates with the frequency of recurrent HSV infections. Thus, in one embodiment, the compositions and methods of the present invention are useful for preventing or inhibiting recurrent HSV infections. In one embodiment, antibodies to e.g. gC-1 block domains involved in immune evasion, which enhances complement activity, improves neutralizing activity of anti-gD-1 IgG, increases antibody- and complement-dependent cellular cytotoxicity, and augments complement-mediated neutralization and lysis of infected cells.

In one embodiment, symptoms are primary, while in another embodiment, symptoms are secondary. In one embodiment, “primary” refers to a symptom that is a direct result of the subject viral infection, while in one embodiment, “secondary” refers to a symptom that is derived from or consequent to a primary cause. In one embodiment, the compositions and strains for use in the present invention treat primary or secondary symptoms or secondary complications related to HSV infection.

In another embodiment, “symptoms” may be any manifestation of an HSV infection, comprising blisters, ulcerations, or lesions on the urethra, cervix, upper thigh, and/or anus in women and on the penis, urethra, scrotum, upper thigh, and anus in men, inflammation, swelling, fever, flu-like symptoms, sore mouth, sore throat, pharyngitis, pain, blisters on tongue, mouth or lips, ulcers, cold sores, neck pain, enlarged lymph nodes, reddening, bleeding, itching, dysuria, headache, muscle pain, etc., or a combination thereof.

In another embodiment, the disease, disorder, or symptom is fever. In another embodiment, the disease, disorder, or symptom is headache. In another embodiment, the disease, disorder, or symptom is stiff neck. In another embodiment, the disease, disorder, or symptom is seizures. In another embodiment, the disease, disorder, or symptom is partial paralysis. In another embodiment, the disease, disorder, or symptom is stupor. In another embodiment, the disease, disorder, or symptom is coma. In another embodiment, the disease, disorder, or symptom is any other disease, disorder, or symptom known in the art that is associated with or secondary to a herpes-mediated encephalitis.

Methods of determining the presence and severity of herpes-mediated encephalitis are well known in the art, and are described, for example, in Bonkowsky J L et al. (Herpes simplex virus central nervous system relapse during treatment of infantile spasms with corticotropin. Pediatrics. 2006 May; 117(5):e1045-8) and Khan O A, et al. (Herpes encephalitis presenting as mild aphasia: case report. BMC Fain Pract. 2006 Mar. 24; 7:22). Each method represents a separate embodiment of the present invention.

In another embodiment, the present invention provides a method of treating or reducing the incidence of a disease, disorder, or symptom associated with an HSV infection in a subject, the method comprising the step of administering to said subject a composition of the present invention.

In another embodiment, the disease, disorder, or symptom secondary to an HSV infection is oral lesions. In another embodiment, the disease, disorder, or symptom is genital lesions. In another embodiment, the disease, disorder, or symptom is oral ulcers. In another embodiment, the disease, disorder, or symptom is genital ulcers. In another embodiment, the disease, disorder, or symptom is fever. In another embodiment, the disease, disorder, or symptom is headache. In another embodiment, the disease, disorder, or symptom is muscle ache. In another embodiment, the disease, disorder, or symptom is swollen glands in the groin area. In another embodiment, the disease, disorder, or symptom is painful urination. In another embodiment, the disease, disorder, or symptom is vaginal discharge. In another embodiment, the disease, disorder, or symptom is blistering. In another embodiment, the disease, disorder, or symptom is flu-like malaise. In another embodiment, the disease, disorder, or symptom is keratitis. In another embodiment, the disease, disorder, or symptom is herpetic whitlow. In another embodiment, the disease, disorder, or symptom is Bell's palsy. In another embodiment, the disease, disorder, or symptom is herpetic erythema multiforme. In another embodiment, the disease, disorder, or symptom is a lower back symptom (e.g. numbness, tingling of the buttocks or the area around the anus, urinary retention, constipation, and impotence). In another embodiment, the disease, disorder, or symptom is a localized eczema herpeticum. In another embodiment, the disease, disorder, or symptom is a disseminated eczema herpeticum. In another embodiment, the disease, disorder, or symptom is a herpes gladiatorum. In another embodiment, the disease, disorder, or symptom is a herpetic sycosis. In another embodiment, the disease, disorder, or symptom is an esophageal symptom (e.g. difficulty swallowing or burning, squeezing throat pain while swallowing, weight loss, pain in or behind the upper chest while swallowing). In another embodiment, the disease, disorder, or symptom is any other disease, disorder, or symptom that is known in the art. Each disease, disorder, and symptom represents a separate embodiment of the present invention.

Thus, in one embodiment, the compositions and methods of the instant invention treat, suppress, inhibit, or reduce the incidence of the infection itself, while in another embodiment, the compositions and methods of the instant invention treat, suppress, inhibit, or reduce the incidence of primary symptoms of the infection, while in another embodiment, the compositions and methods of the instant invention treat, suppress, inhibit, or reduce the incidence of secondary symptoms of the infection. It is to be understood that the compositions and methods of the instant invention may affect any combination of the infection, the primary symptoms caused by the infection, and secondary symptoms related to the infection.

The HSV infection that is treated or ameliorated by methods and compositions of the present invention is, in another embodiment, a genital HSV infection. In another embodiment, the HSV infection is an oral HSV infection. In another embodiment, the HSV infection is an ocular HSV infection. In another embodiment, the HSV infection is a dermatologic HSV infection.

In another embodiment, the present invention provides a method of reducing the incidence of a disseminated HSV infection in a subject, the method comprising the step of administering to said subject a composition of the present invention.

In another embodiment, the present invention provides a method of reducing the incidence of a neonatal HSV infection in an offspring of a subject, the method comprising the step of administering to said subject a composition of the present invention.

In another embodiment, the present invention provides a method of reducing a transmission of an HSV infection from a subject to an offspring thereof, the method comprising the step of administering to said subject a composition of the present invention.

In another embodiment, the offspring is an infant. In another embodiment, the transmission that is reduced or inhibited is transmission during birth. In another embodiment, transmission during breastfeeding is reduced or inhibited. In another embodiment, the transmission that is reduced or inhibited is any other type of parent-to-offspring transmission known in the art.

In another embodiment, the present invention provides a method of reducing a severity of a neonatal HSV infection in an offspring of a subject, the method comprising the step of administering to said subject a composition of the present invention.

In one embodiment, the present invention provides a method of treating, suppressing, inhibiting, or reducing the incidence of an HSV infection in a subject infected with HIV, the method comprising the step of administering to said subject a composition comprising: (a) a modified mRNA encoding HSV gC protein or fragment thereof; (b) a modified mRNA encoding HSV gE protein or fragment thereof; and (c) an adjuvant. In another embodiment, the present invention provides a method of treating, suppressing, inhibiting, or reducing the incidence of an HSV infection in a subject infected with HIV, the method comprising the step of administering to said subject a composition comprising: (a) a modified mRNA encoding HSV gC protein or fragment thereof, wherein said fragment comprises either a C3b-binding domain thereof, a properdin interfering domain thereof, a C5 interfering domain thereof, or a fragment of said C3b-binding domain, properdin interfering domain, or C5-interfering domain; (b) a modified mRNA encoding HSV gE protein or fragment thereof, wherein said fragment comprises AA 24-409 or a fragment thereof; and (c) an adjuvant.

In another embodiment, the present invention provides a method of treating an HSV infection in a subject infected with HIV, the method comprising the step of administering to said subject a composition of the present invention. In another embodiment, the present invention provides a method of suppressing an HSV infection in a subject infected with HIV, the method comprising the step of administering to said subject a composition of the present invention. In another embodiment, the present invention provides a method of inhibiting an HSV infection in a subject infected with HIV, the method comprising the step of administering to said subject a composition of the present invention. In another embodiment, the present invention provides a method of reducing the incidence of an HSV infection in a subject infected with HIV, the method comprising the step of administering to said subject a composition of the present invention. In another embodiment, the present invention provides a method of preventing an HIV infection, the method comprising the step of administering to said subject an HSV composition of the present invention. In one embodiment, HSV infection increases the risk of HIV infection, and protection against HSV infection decreases the risk of HIV infection. Thus, in one embodiment, the present invention provides a method of decreasing the risk of an HIV infection, the method comprising the step of administering to said subject a composition of the present invention.

In one embodiment, the composition for use in the methods of the present invention elicits an immune response against HSV. In another embodiment, the composition for use in the methods of the present invention elicits an immune response against HSV-1. In another embodiment, the composition for use in the methods of the present invention elicits an immune response against HSV-2. In another embodiment, the composition comprises modified mRNAs encoding gD and gC proteins. In another embodiment, the composition comprises modified mRNAs encoding gE and gD proteins. In another embodiment, the composition comprises modified mRNAs encoding gC and gE proteins. In another embodiment, the composition comprises modified mRNAs encoding gE, gD, and gC proteins. In another embodiment, the composition comprises modified mRNAs encoding gE, gD, or gC protein. In another embodiment, the proteins encoded by the modified mRNAs are HSV-1 proteins. In another embodiment, the proteins encoded by the modified mRNAs are HSV-2 proteins. In another embodiment, the proteins encoded by the modified mRNAs comprise both HSV-1 and HSV-2 proteins.

It is to be understood that, in one embodiment, a subject according to any of the embodiments described herein may be a subject infected with, or in another embodiment, susceptible to infection with HSV. In one embodiment, a subject may be infected with, or in another embodiment, susceptible to infection with at least one other pathogen. In one embodiment, a subject may be immunocompromised. In one embodiment, the subject is infected by HSV, while in another embodiment, the subject is at risk for infection by HSV, which in one embodiment, is a subject who is a neonate, in another embodiment, immunocompromised, in another embodiment, elderly, and in another embodiment, an immunocompromised neonate or an immunocompromised elderly subject.

In another embodiment, the compositions of the present invention and their related uses may suppress, inhibit, prevent or treat an HIV infection in a subject. In one embodiment, the compositions of the present invention and their related uses may treat secondary complications of HIV infection, which in one embodiment, are opportunistic infections, neoplasms, neurologic abnormalities, or progressive immunologic deterioration. In another embodiment, the methods comprise treating acquired immunodeficiency syndrome (AIDS). In another embodiment, the methods comprise treating a decline in the number of CD4⁺ T lymphocytes.

In another embodiment, the present invention provides a method of reducing HIV-1 transmission to an offspring, the method comprising the step of administering to a subject a composition of the present invention. As is known in the art, HSV-2 infection increases HIV-1 viral shedding in genital secretions (Nagot N et al., Reduction of HIV-1 RNA levels with therapy to suppress herpes simplex virus. N Engl J Med. 2007 Feb. 22; 356(8):790-9). Thus, methods of the present invention of inhibiting HSV-2 infection are also efficacious for reducing HIV-1 transmission to an offspring. In another embodiment, the mutant HSV strain is an HSV-1 strain. In another embodiment, the mutant HSV strain is an HSV-2 strain.

In another embodiment, the present invention provides a method of reducing HIV-1 transmission to a sexual partner, the method comprising the step of administering to a subject a composition of the present invention. As is known in the art, HSV-2 infection increases HIV-1 viral shedding in genital secretions. Thus, methods of the present invention of inhibiting HSV-2 infection are also efficacious for reducing HIV-1 transmission to a sexual partner. In another embodiment, the mutant HSV strain is an HSV-1 strain. In another embodiment, the mutant HSV strain is an HSV-2 strain.

In another embodiment, the present invention provides a method of reducing susceptibility to HIV-1, the method comprising the step of administering to a subject a composition of the present invention. As is known in the art, HSV-2 infection increases HIV-1 replication (Ouedraogo A et al., Impact of suppressive herpes therapy on genital HIV-1 RNA among women taking antiretroviral therapy: a randomized controlled trial. AIDS. 2006 Nov. 28; 20(18):2305-13). Thus, methods of the present invention of inhibiting HSV-2 infection are also efficacious for reducing susceptibility to HIV-1. In another embodiment, the mutant HSV strain is an HSV-1 strain. In another embodiment, the mutant HSV strain is an HSV-2 strain.

Thus, in one embodiment, the present invention provides a method of inhibiting a primary HSV infection in an HIV-infected subject, comprising the step of administering to said subject a composition of the present invention. In another embodiment, the present invention provides a method of treating or reducing the incidence of an HSV infection in an HIV-infected subject, comprising the step of administering to said subject a composition of the present invention. In another embodiment, the present invention provides a method of inhibiting a flare, recurrence, or HSV labialis following a primary HSV infection in an HIV-infected subject, the method comprising the step of administering to said subject a composition of the present invention. In one embodiment, administration of a composition of the present invention an anti-HSV immune response.

In another embodiment, the present invention provides a method for inducing an immune response in a subject, the method comprising the step of administering to said subject a nucleoside modified mRNA composition of the present invention. In another embodiment, the immune response comprises a CD4 immune response. In another embodiment, the immune response comprises a CD8 immune response. In another embodiment, the immune response comprises a T follicular helper cell immune response. In another embodiment, the immune response comprises a germinal center B cell immune response. In another embodiment, the immune response comprises an IgG antibody response to gC2, gD2, gE2 or a combination thereof.

In another embodiment, the present invention provides a method of treating a Herpes Simplex Virus (HSV) infection in a subject, the method comprising the step of intramuscularly administering to said subject a nucleoside modified mRNA composition of the present invention. In another embodiment, the invention provides a method of suppressing, inhibiting, or reducing the incidence of a Herpes Simplex Virus (HSV) infection in a subject, the method comprising the step of intramuscularly administering to said subject a nucleoside modified mRNA composition of the present invention.

### Administration and Pharmaceutical Regimens

Compositions of the present invention can be, in another embodiment, administered to a subject by any method known to a person skilled in the art, such as parenterally, paracancerally, transmucosally, transdermally, intramuscularly, intravenously, intra-dermally, subcutaneously, intra-peritonealy, intra-ventricularly, intra-cranially, intra-vaginally, intra-nasally, intra-tumorally, or topically.

“Administering,” in another embodiment, refers to directly introducing into a subject by injection or other means a composition of the present invention. In another embodiment, “administering” refers to contacting a cell of the subject's immune system with a composition or modified mRNA encoding HSV protein or mixture thereof.

In another embodiment of the methods and compositions of the present invention, the compositions are administered orally, and are thus formulated in a form suitable for oral administration, i.e. as a solid or a liquid preparation. Suitable solid oral formulations include tablets, capsules, pills, granules, pellets and the like. Suitable liquid oral formulations include solutions, suspensions, dispersions, emulsions, oils and the like. In another embodiment of the present invention, the active ingredient is formulated in a capsule. In accordance with this embodiment, the compositions of the present invention comprise, in addition to the active compound and the inert carrier or diluent, a hard gelating capsule.

In other embodiments, the pharmaceutical compositions are administered by intravenous, intra-arterial, or intramuscular injection of a liquid preparation. Suitable liquid formulations include solutions, suspensions, dispersions, emulsions, oils and the like. In another embodiment, the pharmaceutical compositions are administered intravenously and are thus formulated in a form suitable for intravenous administration. In another embodiment, the pharmaceutical compositions are administered intra-arterially and are thus formulated in a form suitable for intra-arterial administration. In another embodiment, the pharmaceutical compositions are administered intramuscularly and are thus formulated in a form suitable for intramuscular administration.

In another embodiment, the pharmaceutical compositions are administered topically to body surfaces and are thus formulated in a form suitable for topical administration. Suitable topical formulations include gels, ointments, creams, lotions, drops and the like. For topical administration, the compositions or their physiologically tolerated derivatives are prepared and applied as solutions, suspensions, or emulsions in a physiologically acceptable diluent with or without a pharmaceutical carrier.

In another embodiment, the composition is administered as a suppository, for example a rectal suppository or a urethral suppository. In another embodiment, the pharmaceutical composition is administered by subcutaneous implantation of a pellet. In another embodiment, the pellet provides for controlled release of agent over a period of time.

In a preferred embodiment, pharmaceutical compositions are administered intramuscularly, subcutaneously or intradermally.

“Effective dosage” of the modified mRNA, refers, in another embodiment, to an amount sufficient to exert a therapeutic effect. In another embodiment, the term refers to an amount sufficient to elicit expression of a detectable amount of the encoded protein. Each possibility represents a separate embodiment of the present invention.

Methods for measuring the dose of a modified mRNA encoding an HSV glycoprotein (e.g. in human subjects) are well known in the art, and include, for example, dose-escalating trials. Each method represents a separate embodiment of the present invention.

In some embodiments, any of the HSV compositions of and for use in the methods of this invention will comprise a modified mRNA encoding HSV protein or combination of modified mRNAs encoding HSV proteins of the present invention, in any form or embodiment as described herein. In some embodiments, any of the compositions of and for use in the methods will consist of a modified mRNA encoding HSV protein or combination of modified mRNA encodings HSV proteins of the present invention, in any form or embodiment as described herein. In some embodiments, the compositions of this invention will consist essentially of a modified mRNA encoding an HSV protein or combination of modified mRNAs encoding HSV proteins of the present invention, in any form or embodiment as described herein. In some embodiments, the term “comprise” refers to the inclusion of modified mRNA encoding other HSV proteins, as well as inclusion of modified mRNA encoding other proteins that may be known in the art. In some embodiments, the term “consisting essentially of” refers to a composition, which has the modified mRNA encoding a specific HSV protein or fragment thereof. However, other components may be included that are not involved directly in the utility of the modified mRNA(s) encoding HSV protein(s). In some embodiments, the term “consisting” refers to a composition having a modified mRNA encoding particular HSV protein or fragment or combination of modified mRNAs encoding HSV proteins or fragments of the present invention, in any form or embodiment as described herein.

In another embodiment, the present invention provides a composition for treating HSV-1 or a symptom or manifestation thereof, the composition comprising a modified mRNA of the present invention.

In another embodiment, the present invention provides a composition for treating HSV-2 or a symptom or manifestation thereof, the composition comprising a modified mRNA of the present invention.

It is to be understood that the compositions, and methods of the present invention may be used in non-HSV herpesvirus as well, which in one embodiment, proteins gD, gE, or gC proteins that are, in one embodiment, 70% homologous, in another embodiment, 80% homologous, in another embodiment, 85% homologous, in another embodiment, 90% homologous, in another embodiment, 95% homologous, in another embodiment, 98% homologous, and in another embodiment, 100% homologous to the gD, gE, or gC proteins of HSV-1, or in another embodiment, of HSV-2. In one embodiment, such compositions may be useful in suppressing, inhibiting, preventing, or treating, cancers, or in another embodiment, tumors. In one embodiment, non-HSV herpesvirus comprise Varicella Zoster Virus (VZV), Epstein-Barr virus (EBV), EBNA, cytomegalovirus (CMV), and human herpesvirus-6 (HHV-6).

In another embodiment, of methods of the present invention, a composition of the present invention is administered once. In another embodiment, the composition is administered twice. In another embodiment, the composition is administered three times. In another embodiment, the composition is administered four times. In another embodiment, the composition is administered at least four times. In another embodiment, the composition is administered more than four times.

In another embodiment, the dosage is a daily dose. In another embodiment, the dosage is a weekly dose. In another embodiment, the dosage is a monthly dose. In another embodiment, the dosage is an annual dose. In another embodiment, the dose is one is a series of a defined number of doses. In another embodiment, the dose is a one-time dose.

In one embodiment, any of the booster doses described hereinabove is administered following a priming dose comprising one or modified more mRNAs encoding HSV-1 proteins or immunogenic fragments thereof. In another embodiment, any of the booster doses described hereinabove is administered following a priming vaccination comprising one or more modified more mRNAs encoding HSV-2 proteins or immunogenic fragments thereof.

In one embodiment, a subject is immunized with a single administration of the composition. In another embodiment, a subject is immunized with a single dose. In another embodiment, a subject is immunized with two doses. In another embodiment, a subject is immunized with three doses. In another embodiment, a subject is immunized with four doses. In another embodiment, a subject is immunized with five doses.

In one embodiment, all the components of the composition are provided in equal concentrations. According to this aspect and in one embodiment, modified mRNAs encoding gC, gD, and gE are provided in a ratio of 1:1:1. In another embodiment, modified mRNAs encoding gC, gD, and gE are provided in a ratio of 5:2:5. In another embodiment, modified mRNAs encoding gC and gD are provided in a ratio of 1:1. In another embodiment, modified mRNAs encoding gC and gE are provided in a ratio of 1:1. In another embodiment, modified mRNAs encoding gD and gE are provided in a ratio of 1:1.

In one embodiment, modified mRNAs encoding gC, gD, gE, or a combination thereof, or combined with other HSV glycoproteins, are administered in a single composition at the same site and by the same route, while in another embodiment, modified mRNAs encoding gC, gD, and gE are administered in separate compositions at separate sites but by the same route of administration, or in another embodiment, modified mRNAs encoding gC, gD, and gE are administered in separate compositions at separate sites and by different routes of administration, or in another embodiment, modified mRNAs encoding gC, gD, and gE are administered in separate compositions at the same site and by different routes of administration (e.g. injection and topical).

In one embodiment, the methods of the present invention include a one-time or single administration of compositions comprising one or more nucleoside modified mRNAs of the present invention. In another embodiment, the methods of the present invention include administration of compositions comprising one or more nucleoside modified mRNAs in a prime and boost approach. In one embodiment, the methods of the present invention further comprise the step of administering to said subject one or more additional administrations of said nucleoside modified mRNA composition subsequent to the first administration.

In another embodiment, the methods of the present invention comprise administering a composition comprising one or more nucleoside modified mRNAs encoding one or more HSV glycoproteins as a first administration and a composition comprising one or more HSV glycoproteins as a second or subsequent administration. In one embodiment, the HSV glycoproteins encoded by the mRNA in the first (or prime) administration are the same glycoproteins in the second or subsequent (or boost) administration. In another embodiment, a composition comprising one or more HSV glycoproteins is administered as a first administration, and a composition comprising one or more nucleoside modified mRNAs encoding one or more HSV glycoproteins is administered as a second or subsequent administration. Each possibility represents a separate embodiment of the present invention.

In another embodiment, modified mRNAs encoding gC, gD, and gE are administered simultaneously followed by a booster dose of modified mRNA encoding gD without modified mRNAs encoding gC or gE. In another embodiment, modified mRNAs encoding gC, gD, and gE are administered simultaneously followed by a booster dose of modified mRNA encoding gC without modified mRNAs encoding gD or gE. In another embodiment, modified mRNAs encoding gC, gD, and gE are administered simultaneously followed by a booster dose of modified mRNA encoding gE without modified mRNAs encoding gD or gC. In another embodiment, modified mRNAs encoding gC, gD, and gE are administered simultaneously followed by a booster dose of modified mRNAs encoding gC and gD without modified mRNAs encoding gE. In another embodiment, modified mRNAs encoding gC, gD, and gE are administered simultaneously followed by a booster dose of modified mRNAs encoding gC and gE without modified mRNAs encoding gD. In another embodiment, modified mRNAs encoding gC, gD, and gE are administered simultaneously followed by a booster dose of modified mRNAs encoding gD and gE without modified mRNAs encoding gE. In one embodiment the booster administration is performed at the same site and by the same mode of administration as the priming administration. In another embodiment, the booster administration is performed at a different site from the priming administration but by the same mode of administration as the priming administration. In one embodiment the booster administration is performed at the same site but by different mode of administration than priming administration. In another embodiment, the booster administration is performed at a different site and by different mode of administration than priming administration.

In one embodiment, the modified mRNA induces a detectably lower innate immune response than the same quantity of unmodified RNA having the same sequence.

In one embodiment, the effectiveness of the compositions and methods of the present invention are dependent on the presence of complement, while in another embodiment, the compositions and methods of the present invention are not dependent on the presence of complement. In one embodiment, the effectiveness of some of the compositions for use in the methods of the present invention are dependent on the presence of complement, while others are not. In one embodiment, the anti-gC antibody is dependent on complement for its effectiveness against HSV.

In one embodiment, complement is an important contributor to innate and acquired immunity. In one embodiment, complement activation facilitates virus neutralization by particle phagocytosis and lysis, functions as a chemoattractant for neutrophils and macrophages, and enhances B and T cell responses. In one embodiment, HSV-1 gC binds complement C3b and blocks C5 and properdin interaction with C3b, which inhibit complement activation and complement-mediated virus neutralization. In one embodiment, a gC-1 domain that interacts with complement is located within amino acids 33 to 133 and blocks C5 and properdin binding to C3b, and in one embodiment, a gC-1 domain that interacts with complement extends from amino acids 124 to 366 and directly binds C3b. In one embodiment, an HSV-1 gC mutant virus deleted in the C3b binding domain is more susceptible to complement-mediated virus neutralization in vitro and less virulent than wild-type (WT) virus in the mouse flank model. Therefore, in one embodiment, the interaction between gC-1 and C3b enhances HSV-1 virulence, and in one embodiment, blocking the gC-1 domain is effective in preventing or treating HSV-1 infection.

In one embodiment, the compositions and methods of the present invention are for use in human subjects, while in another embodiment, they are for use in animal subjects. In another embodiment, the subject is mammalian. In another embodiment, the subject is any organism susceptible to infection by HSV. In one embodiment, the subject is murine, bovine, ovine, canine, feline, equine, porcine, etc. In one embodiment, the compositions and methods of the present invention are effective in male subjects. In another embodiment, the compositions and methods of the present invention are effective in female subjects. In one embodiment, the compositions and methods of the present invention are effective in seronegative subjects. In another embodiment, the compositions and methods of the present invention are effective in seropositive subjects.

### Pharmaceutical Formulations

In one embodiment, a method of present invention further comprises mixing the modified mRNA with a transfection reagent prior to the step of contacting. In another embodiment, a method of present invention further comprises administering the modified mRNA together with the transfection reagent. In another embodiment, the transfection reagent is a cationic lipid reagent.

In another embodiment, the transfection reagent is a lipid-based transfection reagent. In another embodiment, the transfection reagent is a protein-based transfection reagent. In another embodiment, the transfection reagent is a polyethyleneimine based transfection reagent. In another embodiment, the transfection reagent is calcium phosphate. In another embodiment, the transfection reagent is Lipofectin® or Lipofectamine®. In another embodiment, the transfection reagent is any other transfection reagent known in the art.

In another embodiment, the transfection reagent forms a liposome. Liposomes, in another embodiment, increase intracellular stability, increase uptake efficiency and improve biological activity.

In another embodiment, liposomes are hollow spherical vesicles composed of lipids arranged in a similar fashion as those lipids which make up the cell membrane. They have, in another embodiment, an internal aqueous space for entrapping water soluble compounds and range in size from 0.05 to several microns in diameter. In another embodiment, liposomes can deliver RNA to cells in a biologically active form (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid).

Each type of transfection reagent represents a separate embodiment of the present invention.

In another embodiment, a modified mRNA of the present invention is encapsulated in a nanoparticle. Methods for nanoparticle packaging are well known in the art, and are described, for example, in Bose S, et al (Role of Nucleolin in Human Parainfluenza Virus Type 3 Infection of Human Lung Epithelial Cells. J. Virol. 78:8146. 2004); Dong Y et al. Poly(d,l-lactide-co-glycolide)/montmorillonite nanoparticles for oral delivery of anticancer drugs. Biomaterials 26:6068. 2005); Lobenberg R. et al (Improved body distribution of 14C-labelled AZT bound to nanoparticles in rats determined by radioluminography. J Drug Target 5:171.1998); Sakuma S R et al (Mucoadhesion of polystyrene nanoparticles having surface hydrophilic polymeric chains in the gastrointestinal tract. Int J Pharm 177:161. 1999); Virovic L et al. Novel delivery methods for treatment of viral hepatitis: an update. Expert Opin Drug Deliv 2:707.2005); and Zimmermann E et al, Electrolyte- and pH-stabilities of aqueous solid lipid nanoparticle (SLN) dispersions in artificial gastrointestinal media. Eur J Pharm Biopharm 52:203. 2001). Each method represents a separate embodiment of the invention.

In one embodiment, WmRNA is encapsulated in nanoparticles to improve efficiency of delivery and expression of WmRNA. Nanoparticle packaging involves condensing and encapsulating RNA into particles that are smaller than the pore of the nuclear membrane, using chemicals including poly-L-lysine and polyethylene glycol. In one embodiment, RNA is packaged into one of four nanoparticle formulations (PEI, PLL, PAE, and CK₃₀PEG₁₀ₖ).

### Lipid Nanoparticles

In one embodiment, nanoparticles used in the compositions and methods of the present invention comprise lipid nanoparticles as described in Cullis, P., & Hope, M. (n.d.). Lipid Nanoparticle Systems for Enabling Gene Therapies. Molecular therapy., 25(7), which is incorporated by reference herein in its entirety.

In one embodiment, delivery of nucleoside-modified RNA comprises any suitable delivery method, including exemplary RNA transfection methods described elsewhere herein. In certain embodiments, delivery of a nucleoside-modified RNA to a subject comprises mixing the nucleoside-modified RNA with a transfection reagent prior to the step of contacting. In another embodiment, a method of present invention further comprises administering nucleoside-modified RNA together with the transfection reagent. In another embodiment, the transfection reagent is a cationic lipid reagent.

In another embodiment, the transfection reagent is a lipid-based transfection reagent. In another embodiment, the transfection reagent is a protein-based transfection reagent. In another embodiment, the transfection reagent is a polyethyleneimine based transfection reagent. In another embodiment, the transfection reagent is calcium phosphate. In another embodiment, the transfection reagent is Lipofectin®, Lipofectamine®, or TransIT®. In another embodiment, the transfection reagent is any other transfection reagent known in the art.

In another embodiment, the transfection reagent forms a liposome.

Liposomes, in another embodiment, increase intracellular stability, increase uptake efficiency and improve biological activity. In another embodiment, liposomes are hollow spherical vesicles composed of lipids arranged in a similar fashion as those lipids which make up the cell membrane. They have, in another embodiment, an internal aqueous space for entrapping water-soluble compounds and range in size from 0.05 to several microns in diameter. In another embodiment, liposomes can deliver RNA to cells in a biologically active form.

In one embodiment, the composition comprises a lipid nanoparticle (LNP) and one or more nucleic acid molecules described herein. For example, in one embodiment, the composition comprises an LNP and one or more nucleoside-modified RNA molecules encoding one or more antigens, adjuvants, or a combination thereof.

The term “lipid nanoparticle” refers to a particle having at least one dimension on the order of nanometers (e.g., 1-1,000 nm) which includes one or more lipids, for example a lipid of Formula (I), (II) or (III), as described in WO2016176330A1, which is incorporated by reference herein in its entirety.

In some embodiments, lipid nanoparticles are included in a formulation comprising a nucleoside-modified RNA as described herein. In some embodiments, such lipid nanoparticles comprise a cationic lipid and one or more excipient selected from neutral lipids, charged lipids, steroids and polymer conjugated lipids (e.g., a pegylated lipid such as a pegylated lipid of structure (IV), such as compound IVa). In some embodiments, the nucleoside-modified RNA is encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells e.g. an adverse immune response.

In various embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic. In certain embodiments, the nucleoside-modified RNA, when present in the lipid nanoparticles, is resistant in aqueous solution to degradation with a nuclease.

The LNP may comprise any lipid capable of forming a particle to which the one or more nucleic acid molecules are attached, or in which the one or more nucleic acid molecules are encapsulated. The term “lipid” refers to a group of organic compounds that are derivatives of fatty acids (e.g., esters) and are generally characterized by being insoluble in water but soluble in many organic solvents. Lipids are usually divided in at least three classes: (1) “simple lipids” which include fats and oils as well as waxes; (2) “compound lipids” which include phospholipids and glycolipids; and (3) “derived lipids” such as steroids.

In one embodiment, the LNP comprises one or more cationic lipids, and one or more stabilizing lipids. Stabilizing lipids include neutral lipids and pegylated lipids.

In one embodiment, the LNP comprises a cationic lipid. As used herein, the term “cationic lipid” refers to a lipid that is cationic or becomes cationic (protonated) as the pH is lowered below the pK of the ionizable group of the lipid, but is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease.

In certain embodiments, the cationic lipid comprises any of a number of lipid species which carry a net positive charge at a selective pH, such as physiological pH. Such lipids include, but are not limited to, N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3-dioleoyloxy)propyl)-N,N,N-trimethylammonium chloride (DOTAP); 3-(N—(N′,N′-dimethylaminoethane)-carbamoyl)cholesterol (DC-Choi), N-(1-(2,3-dioleoyloxy)propyl)-N-2-(sperminecarboxamido)ethyl)-N,N-dimethylammonium trifluoracetate (DOSPA), dioctadecylamidoglycyl carboxy spermine (DOGS), 1,2-dioleoyl-3-dimethylammonium propane (DODAP), N,N-dimethyl-2,3-dioleoyloxy)propylamine (DODMA), and N-(1,2-dimyristyloxyprop-3-yl)-N,N-dimethyl-N-hydroxyethyl ammonium bromide (DMRIE). Additionally, a number of commercial preparations of cationic lipids are available which can be used in the present invention. These include, for example, LIPOFECTIN® (commercially available cationic liposomes comprising DOTMA and 1,2-dioleoyl-sn-3-phosphoethanolamine (DOPE), from GIBCO/BRL, Grand Island, N.Y.); LIPOFECTAMINE® (commercially available cationic liposomes comprising N-(1-(2,3-dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.). The following lipids are cationic and have a positive charge at below physiological pH:

DODAP, DODMA, DMDMA, 1,2-dilinoleyloxy-N,N-dimethylaminopropane (DLinDMA), 1,2-dilinolenyloxy-N,N-dimethylaminopropane (DLenDMA).

In one embodiment, the cationic lipid is an amino lipid. Suitable amino lipids useful in the invention include those described in WO 2012/016184, incorporated herein by reference in its entirety. Representative amino lipids include, but are not limited to, 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA).

In certain embodiments, the cationic lipid is present in the LNP in an amount from about 30 to about 95 mole percent. In one embodiment, the cationic lipid is present in the LNP in an amount from about 30 to about 70 mole percent. In one embodiment, the cationic lipid is present in the LNP in an amount from about 40 to about 60 mole percent. In one embodiment, the cationic lipid is present in the LNP in an amount of about 50 mole percent. In one embodiment, the LNP comprises only cationic lipids. In certain embodiments, the L P comprises one or more additional lipids which stabilize the formation of particles during their formation.

Suitable stabilizing lipids include neutral lipids and anionic lipids.

The term “neutral lipid” refers to any one of a number of lipid species that exist in either an uncharged or neutral zwitterionic form at physiological pH.

Representative neutral lipids include diacylphosphatidylcholines, diacylphosphatidylethanolamines, ceramides, sphingomyelins, dihydro sphingomyelins, cephalins, and cerebrosides.

Exemplary neutral lipids include, for example, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoyl-phosphatidyethanol amine (SOPE), and 1,2-dielaidoyl-sn-glycero-3-phophoethanolamine (transDOPE). In one embodiment, the neutral lipid is 1,2-distearoyl-sn-glycero-3-phosphocholine (DSPC).

In some embodiments, the LNPs comprise a neutral lipid selected from DSPC, DPPC, DMPC, DOPC, POPC, DOPE and SM. In various embodiments, the molar ratio of the cationic lipid (e.g., lipid of Formula (I)) to the neutral lipid ranges from about 2:1 to about 8:1.

In various embodiments, the LNPs further comprise a steroid or steroid analogue.

In certain embodiments, the steroid or steroid analogue is cholesterol. In some of these embodiments, the molar ratio of the cationic lipid (e.g., lipid of Formula (I)) to cholesterol ranges from about 2:1 to 1:1.

The term “anionic lipid” refers to any lipid that is negatively charged at physiological pH. These lipids include phosphatidylglycerol, cardiolipin, diacylphosphatidylserine, diacylphosphatidic acid, N-dodecanoylphosphatidylethanolamines, N-succinylphosphatidylethanolamines, N-glutarylphosphatidylethanolamines, lysylphosphatidylglycerols, palmitoyloleyolphosphatidylglycerol (POPG), and other anionic modifying groups joined to neutral lipids.

In certain embodiments, the LNP comprises glycolipids (e.g., monosialoganglioside GMi). In certain embodiments, the LNP comprises a sterol, such as cholesterol.

In some embodiments, the LNPs comprise a polymer conjugated lipid. The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-s-DMG) and the like.

In certain embodiments, the LNP comprises an additional, stabilizing—lipid which is a polyethylene glycol-lipid (pegylated lipid). Suitable polyethylene glycol-lipids include PEG-modified phosphatidylethanolamine, PEG-modified phosphatidic acid, PEG-modified ceramides (e.g., PEG-CerC14 or PEG-CerC20), PEG-modified dialkylamines, PEG-modified diacylglycerols, PEG-modified dialkylglycerols.

Representative polyethylene glycol-lipids include PEG-c-DOMG, PEG-c-DMA, and PEG-s-DMG. In one embodiment, the polyethylene glycol-lipid is N-[(methoxy poly(ethylene glycol)₂₀₀₀)carbamyl]-1,2-dimyristyloxlpropyl-3-amine (PEG-c-DMA). In one embodiment, the polyethylene glycol-lipid is PEG-c-DOMG). In other embodiments, the LNPs comprise a pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O-(co-methoxy(polyethoxy)ethyl)butanedioate (PEG-S-DMG), a pegylated ceramide (PEG-cer), or a PEG dialkoxypropylcarbamate such as Q-methoxy(polyethoxy)ethyl-N-(2,3-di(tetradecanoxy)propyl)carbamate or 2,3-di(tetradecanoxy)propyl-N-(co-methoxy(polyethoxy)ethyl)carbamate. In various embodiments, the molar ratio of the cationic lipid to the pegylated lipid ranges from about 100:1 to about 25:1.

In certain embodiments, the additional lipid is present in the LNP in an amount from about 1 to about 10 mole percent. In one embodiment, the additional lipid is present in the LNP in an amount from about 1 to about 5 mole percent. In one embodiment, the additional lipid is present in the LNP in about 1 mole percent or about 1.5 mole percent.

In certain embodiments, the LNP comprises one or more targeting moieties which are capable of targeting the LNP to a cell or cell population. For example, in one embodiment, the targeting moiety is a ligand which directs the LNP to a receptor found on a cell surface.

In certain embodiments, the LNP comprises one or more internalization domains. For example, in one embodiment, the LNP comprises one or more domains which bind to a cell to induce the internalization of the LNP. For example, in one embodiment, the one or more internalization domains bind to a receptor found on a cell surface to induce receptor-mediated uptake of the LNP. In certain embodiments, the LNP is capable of binding a biomolecule in vivo, where the LNP-bound biomolecule can then be recognized by a cell-surface receptor to induce internalization. For example, in one embodiment, the LNP binds systemic ApoE, which leads to the uptake of the LNP and associated cargo.

Other exemplary LNPs and their manufacture are described in the art, for example in WO2016176330A1, U.S. Patent Application Publication No. US20120276209, Semple et al., 2010, Nat Biotechnol., 28(2): 172-176; Akinc et al., 2010, Mol Ther., 18(7): 1357-1364; Basha et al., 2011, Mol Ther, 19(12): 2186-2200; Leung et al., 2012, J Phys Chem C Nanomater Interfaces, 116(34): 18440-18450; Lee et al., 2012, Int J Cancer., 131(5): E781-90; Belliveau et al., 2012, Mol Ther nucleic Acids, 1: e37; Jayaraman et al., 2012, Angew Chem Int Ed Engl., 51(34): 8529-8533; Mui et al., 2013, Mol Ther Nucleic Acids. 2, e139; Maier et al., 2013, Mol Ther., 21(8): 1570-1578; and Tarn et al., 2013, Nanomedicine, 9(5): 665-74, each of which are incorporated by reference in their entirety.

In another embodiment, methods of the present invention comprise administering a modified mRNAs encoding HSV glycoprotein and a pharmaceutically acceptable carrier or diluent. In other embodiments, pharmaceutically acceptable carriers for liquid formulations may be aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of petroleum, animal, vegetable, or synthetic origin, for example, peanut oil, soybean oil, mineral oil, olive oil, sunflower oil, and fish-liver oil.

As used herein “pharmaceutically acceptable carriers or diluents” are well known to those skilled in the art.

In another embodiment, the pharmaceutical compositions provided herein are controlled-release compositions, i.e. compositions in which the compound is released over a period of time after administration. Controlled- or sustained-release compositions include formulation in lipophilic depots (e.g. fatty acids, waxes, oils). In another embodiment, the composition is an immediate-release composition, i.e. a composition in which the entire compound is released immediately after administration.

Each of the additives, excipients, formulations and methods of administration represents a separate embodiment of the present invention.

In another embodiment, the present invention provides a kit comprising a reagent utilized in performing a method of the present invention. In another embodiment, the present invention provides a kit comprising a composition, tool, or instrument of the present invention.

The following examples are presented in order to more fully illustrate the preferred embodiments of the invention. They should in no way be construed, however, as limiting the broad scope of the invention.

### EXPERIMENTAL DETAILS SECTION

### Example 1: Materials and Experimental Methods

Modified RNA expressing exemplary HSV-2 glycoproteins C, D and E (gC2/gD2/gE2) ectodomains. Exemplary modified RNA (encoding an immunogenic gC2 fragment (SEQ ID NO: 10), encoding an immunogenic gD2 fragment (SEQ ID NO: 4), and encoding an immunogenic gE2 fragment (SEQ ID NO: 16)) was prepared based on the DNA coding sequences that encode HSV-2 glycoprotein C (gC2; amino acids 27-426 from HSV-2 strain 333; SEQ ID NO: 11), glycoprotein D (gD2; amino acids 26-331 from HSV-2 strain 333; SEQ ID NO: 5), and glycoprotein E (gE2; amino acids 24-405 from HSV-2 strain 2.12; SEQ ID NO: 17).

The exemplary modified RNA was incorporated into lipid nanoparticles (LNP) to prepare the following immunogens: (a) Poly C RNA in LNP; (b) a modified RNA encoding an exemplary immunogenic gC2 fragment in LNP; (c) a modified RNA encoding an exemplary immunogenic gD2 fragment in LNP; (d) a modified RNA encoding an exemplary immunogenic gE2 fragment in LNP; (e) modified RNA encoding exemplary immunogenic gC2, gD2, and gE2 fragments in LNP.

Immunization groups were as follows:

Controls (Poly C group): 10 μg Poly C RNA/LNP divided into 4 aliquots and administered at 4 separate sites.

Exemplary gD2 immunogenic fragment alone (gD2 group): 10 μg RNA encoding an exemplary immunogenic gD2 fragment/LNP divided into 4 aliquots and administered at 4 separate sites.

Individual trivalent (Trivalent-I group): 10 μg RNA encoding an exemplary immunogenic gC2 fragment/LNP, 10 μg RNA encoding an exemplary immunogenic gD2 fragment/LNP, 10 μg RNA encoding an exemplary immunogenic gE2 fragment/LNP each divided into 2 aliquots and each given at 2 sites.

Combined trivalent (Trivalent-C group): 10 μg RNA encoding an exemplary immunogenic gC2 fragment & 10 μg RNA encoding an exemplary immunogenic gD2 fragment & 10 μg RNA encoding an exemplary immunogenic gE2 fragment combined into LNP and divided into 4 aliquots and given at 4 sites.

Experimental Procedures. Hair was removed from the back of 6-8 week old BALB/c mice using an electric razor and Nair. Mice were bled prior to the first and second immunization and prior to intravaginal challenge. Two immunizations were performed intradermally at 28-day intervals. Intradermal immunizations were performed on the denuded backs. Five mice that received the trivalent vaccine at individual sites (group c above) were sacrificed 14 days after the second immunization. Spleens were harvested for CD4⁺ and CD8⁺ T cell responses to exemplary gC2, gD2 and gE2 subunit immunogenic antigen fragments or to 15 amino acid peptides each with 11 overlapping amino acids. Twenty-eight days after the second immunization, mice were treated subcutaneously with 2 mg Depo-Provera and 5 days later infected intravaginally with 5×10³ PFU HSV-2 strain MS (˜400 LD₅₀). On days 2 and 4 post-challenge, vaginal swabs were obtained for virus cultures. On day 4 post-challenge, some mice in each vaccine group were sacrificed and dorsal root ganglia (DRG) excised for HSV-2 DNA qPCR. The remaining animals were evaluated for weight loss and hind limb weakness for 10 days while survival and genital disease were monitored for 28 days.

### Example 2: Characterization of Translational Products Produced by Modified RNA Encoding gC2, gD2, and gE2 Immunogenic Fragments

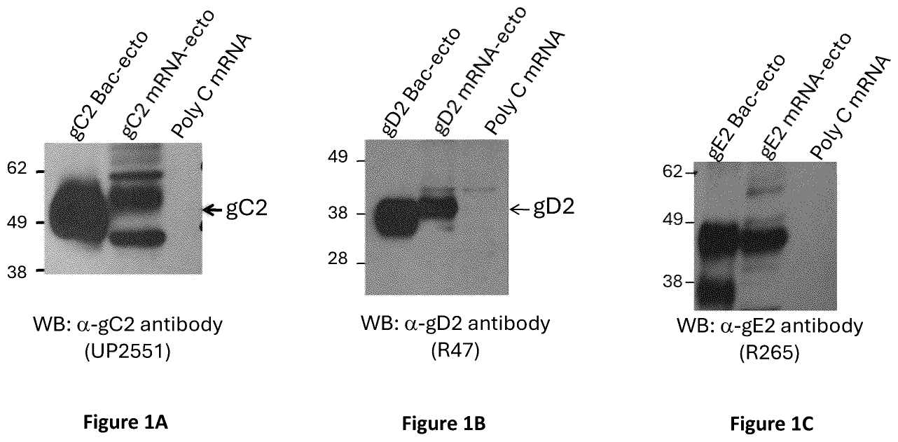

The ability of modified RNA to express proteins of the expected molecular weight when transfected into mammalian cells was verified. 0.1p g of modified RNA encoding exemplary gC2, gD2, or gE2 immunogenic fragments was transfected into 293T cells using TransIT-mRNA (Mirus Bio LLC) for the transfection. Eighteen hours later, cells were harvested and extracts prepared for Western blots. The RNAs were designed to express immunogenic fragments of exemplary gC2, gD2 and gE2 ectodomain (labeled mRNA-ecto). As controls for the expected molecular weights, purified baculovirus proteins gC2, gD2, and gE2 expressing the same amino acids as the RNA constructs (labeled Bac-ecto) were used (FIGS. 1A-C).

Conclusion: When transfected into mammalian cells, modified RNA encoding immunogenic fragments of HSV-2 gC2 (FIG. 1A), gD2 (FIG. 1B) and gE2 (FIG. 1C) ectodomains produced proteins of the appropriate molecular weights that reacted with antibodies to the glycoproteins on Western blot.

### Example 3: ELISA Antibody Responses in Subjects Immunized with gD2 Immunogenic Fragment or Trivalent Modified RNA Vaccines

ELISA endpoint titers were evaluated on sera taken 28 days after the first and second immunizations. Immunization groups were as follows: Poly C (10 μg Poly C RNA/LNP divided into 4 aliquots and administered at 4 separate sites) (control); gD2 (10 μg RNA encoding exemplary gD2 immunogenic fragment/LNP divided into 4 aliquots and administered at 4 separate sites); Trivalent-I (10 μg RNA encoding exemplary gC2 immunogenic fragment/LNP, 10 μg RNA encoding exemplary gD2 immunogenic fragment/LNP, 10 μg RNA encoding exemplary gE2 immunogenic fragment/LNP each divided into 2 aliquots and each given at 2 sites); and Trivalent-C(10 μg RNA encoding exemplary gC2 immunogenic fragment & 10 μg RNA encoding exemplary gD2 immunogenic fragment & 10 μg RNA encoding exemplary gE2 immunogenic fragment combined into LNP and divided into 4 aliquots and given at 4 sites).

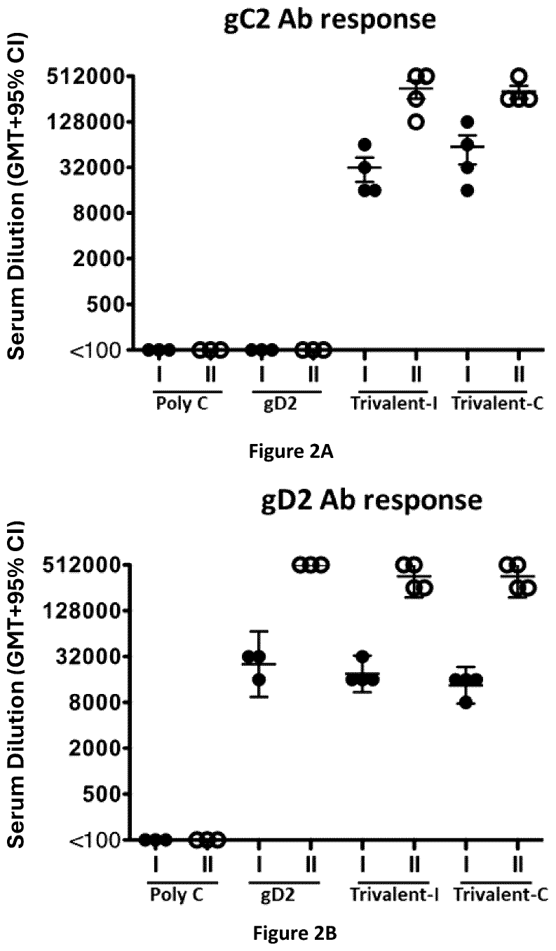

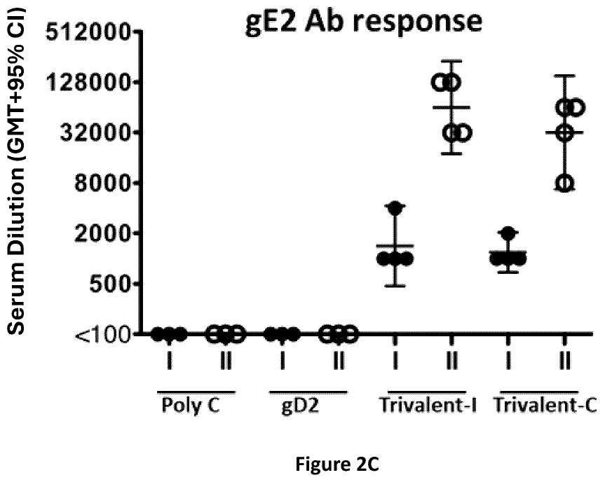

Four animals were evaluated in each group. High ELISA titers to each immunogen were obtained after the first immunization (marked as roman numeral I; FIGS. 2A-C), and the titers were boosted even higher after the second immunization (marked as roman numeral II; FIGS. 2A-C). Immunization with gD2 vaccine selectively induced extremely high titers of ELISA antibodies to gD2 (FIG. 2B), while immunization with the trivalent modified RNA vaccines induced extremely high titers of ELISA antibodies to gC2 (FIG. 2A) and gD2 (FIG. 2B) and high titers to gE2 (FIG. 2C). In all non-control groups, the second immunization significantly boosted the ELISA titers compared to the first. The differences between the titers in the second vs the first immunizations were significant, p<0.05 (t-tests, comparing the antibody titers after the first and second immunization).

Conclusion: The RNA vaccine encoding gD2 and the trivalent RNA vaccines induced extremely high titers of ELISA antibodies after the first immunization that were significantly boosted after the second immunization.

### Example 4: Balanced Th1 and Th2 IgG Isotypes Produced by Modified RNA Immunization

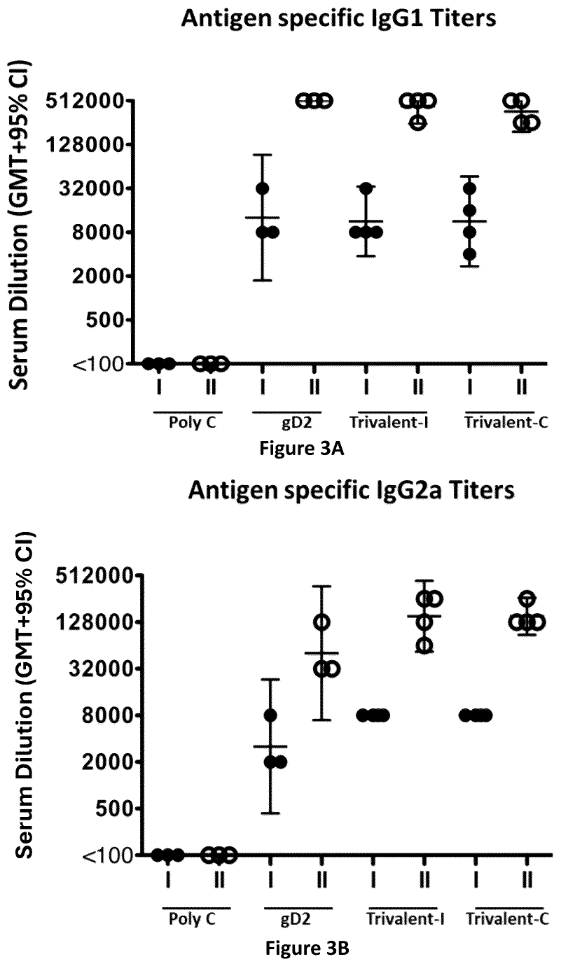

The ability of RNA immunizations to stimulate predominantly a T_{H}1 or T_{H}2 immune response was tested by determining whether IgG1 (T_{H}2) or IgG2a (T_{H}1) antibodies are produced. ELISA was performed on plates coated with all three antigens, gC2, gD2 and gE2. Serum obtained after the first or second immunization was added to the antigen-coated plates, and IgG1 or IgG2a was detected using HRP anti-mouse IgG1 or IgG2a. IgG1 (FIG. 3A) and IgG2a (FIG. 3B) titers were significantly elevated after immunization with the gD2 vaccine and the trivalent modified RNA vaccines. Further, the IgG1 (FIG. 3A) or IgG2a (FIG. 3B) titers were significantly higher after the second modified RNA immunization compared to the first, p<0.05 (t tests).

Conclusion: The results demonstrate high titers of antibodies are produced to both IgG1 and IgG2a isotypes, indicating a balanced T_{H}1 and T_{H}2 response to immunization with modified RNA encoding exemplary gC2, gD2 and gE2 immunogenic fragments.

### Example 5: High Neutralizing Antibody Titers after Modified RNA Immunization

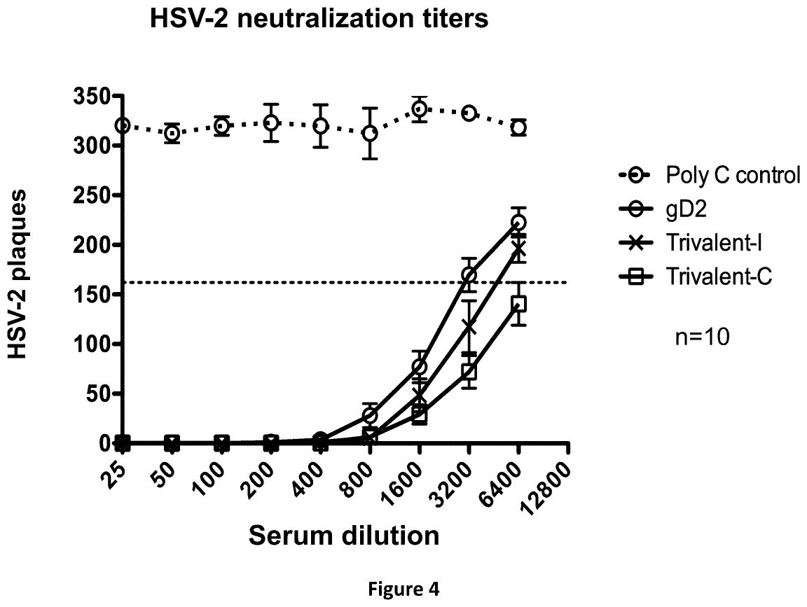

Serum was obtained 28 days after the second immunization, and neutralizing antibody titers were determined using serial 2-fold dilutions of serum, starting at a 1:25 dilution and 10% human serum as a source of complement. The human serum was obtained from an individual seronegative for HSV-1 and HSV-2. The modified RNA groups were each significantly different from the poly C controls (p<0.001; FIG. 4). While each of the RNA groups was not significantly different from one another, the trivalent vaccine given as a combined immunogen (Trivalent-C) performed the best of the three RNA groups (FIG. 4).

Conclusion: Each of the modified RNA groups produced extremely high titers of neutralizing antibodies in the presence of 10% human complement.

### Example 6: CD4+ and CD8+ T Cell Responses in Splenocytes after Modified RNA Immunization

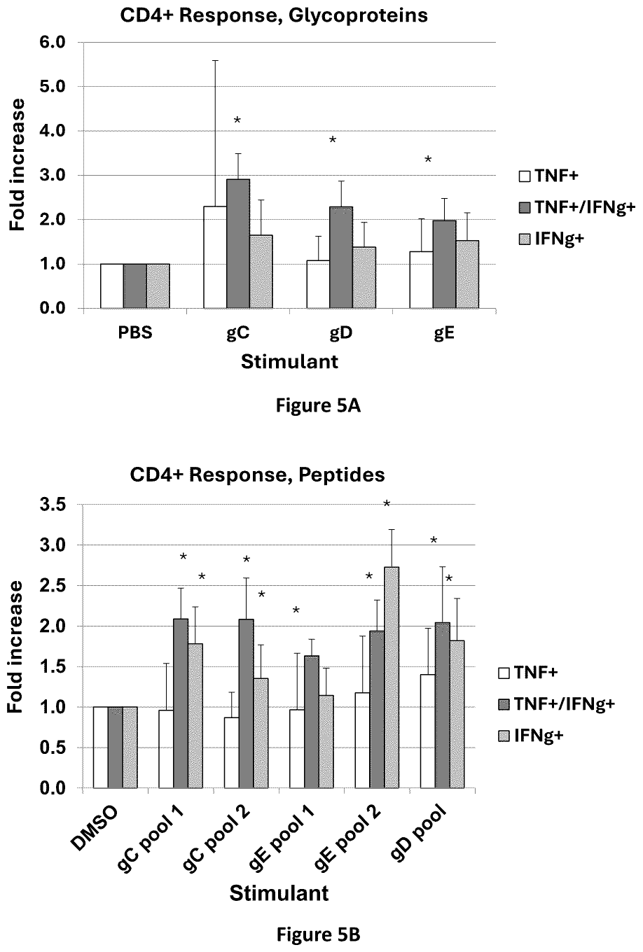

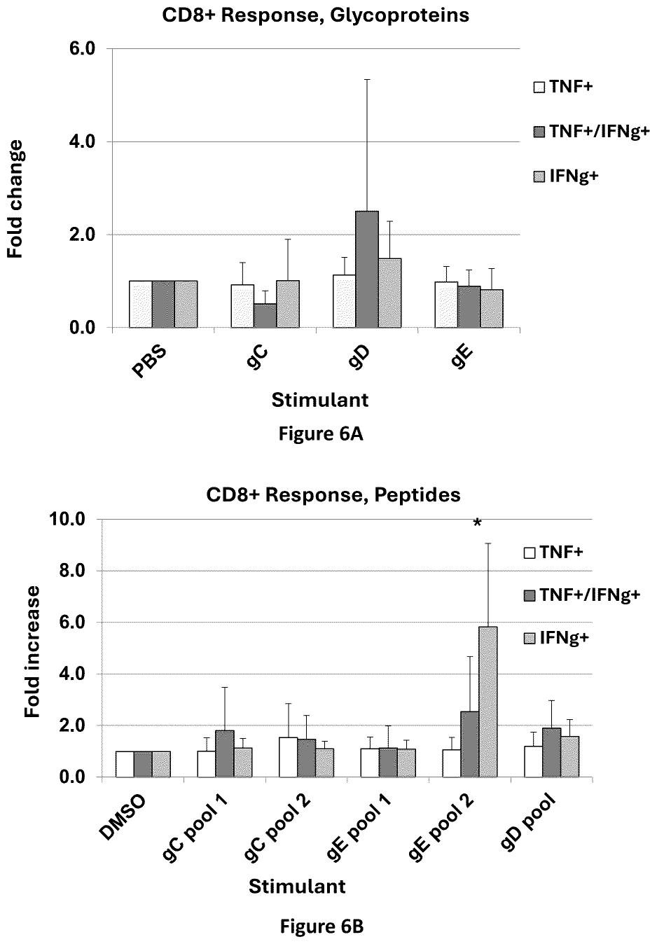

Five animals from the trivalent modified RNA group that were immunized with each exemplary glycoprotein immunogenic fragment RNA at a separate site (Trivalent-I group) were euthanized 14 days after the second immunization. Splenocytes were prepared for T cell assays. Splenocytes were stimulated with glycoprotein subunit antigens prepared in baculovirus or 15 amino acid peptides containing 11 overlapping amino acids. The CD4⁺ and CD8⁺ T cell responses are shown in FIGS. 5A-5B and FIGS. 6A-6B, respectively.

CD4⁺ T cells: The modified RNA-expressed exemplary gC2, gD2, and gE2 subunit immunogenic fragments each stimulated polyfunctional CD4⁺ T cell responses (FIGS. 5A-5B). Splenocytes harvested from immunized subjects and then stimulated with subunit glycoprotein immunogenic fragments increased polyfunctional CD4⁺ T cell responses (FIG. 5A). Splenocytes harvested from immunized subjects and then stimulated with 15 amino acid overlapping peptides increased polyfunctional CD4⁺ T cell responses and IFNγ responses (FIG. 5B).

CD8⁺ T cells: Only exemplary gE immunogenic peptide pool 2 stimulated a significant IFNγ CD8⁺ T cell response (FIGS. 6A-6B).

### Example 7: Survival, Weight Loss and Neurological Signs after Modified RNA Immunization and Intravaginal Challenge

Thirty-three days after the second immunization, animals were inoculated intravaginally with 5×10³ PFU of HSV-2 strain MS (˜400 LD₅₀). Animals were observed daily for survival, neurological signs consisting of hind limb weakness or paralysis and hunched gait, and for weight loss or gain. All animals in the poly C control group died, while all animals in the gD2 vaccine alone, trivalent given individually (labeled Trivalent-I) or trivalent given combined (labeled Trivalent-C) survived (FIG. 7A; p=0.002 by Log-rank (Mantel-Cox) comparing the three exemplary RNA/LNP groups with poly C controls). FIG. 7B demonstrates that administration of the modified RNA vaccine twice at 28 day intervals and challenged intravaginally with HSV-2 does not result in neurological signs or weight loss. Control subjects that were not administered the vaccine and were challenged intravaginally with HSV-2 showed weight loss and neurological signs.

Each of the RNA/LNP groups significantly outperformed the control group. All mice immunized with the modified RNA survived and showed no evidence of weight loss, neurological disease or genital lesions after intravaginal challenge with ˜400 LD₅₀ of HSV-2.

### Example 8: HSV-2 Vaginal Titers after Modified RNA Immunization and Intravaginal Challenge

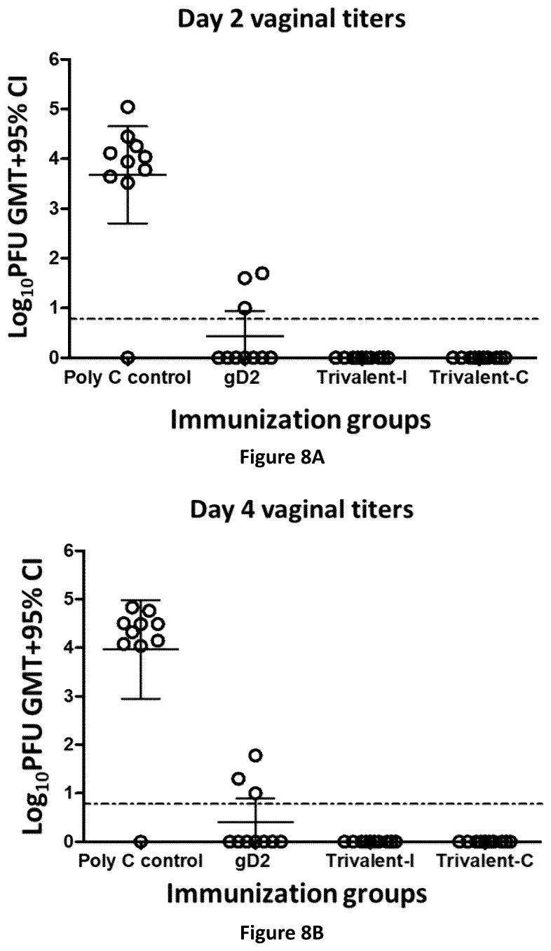

Vaginal swabs were obtained from 10 animals per group on days 2 and 4 post challenge and cultured for replication competent HSV-2 virus. Results are shown in FIGS. 8A-8B. 9/10 animals in the poly C group had positive cultures on days 2 (FIG. 8A) and 4 (FIG. 8B) compared with 3/10 in the gD2 vaccine group and 0/10 in the trivalent-I or trivalent-C groups (P values by Fisher Exact test were not significant comparing trivalent groups to gD2 vaccine group; p<0.001 comparing trivalent-I or trivalent-C with poly C; p=0.02 comparing gD2 vaccine with poly C).

Each of the RNA/LNP groups significantly outperformed the Poly C control group. Remarkably, day 2 and day 4 vaginal titers after challenge were negative in mice immunized with the trivalent RNA whether given at separate sites or as a combined immunization. No significant differences were detected comparing either the trivalent group with the gD2 vaccine group, although both trivalent groups outperformed the gD2 group, as 3 of 10 mice in the gD2 group had virus isolated from vaginal swabs.

### Example 9: Genital Disease after Modified RNA Immunization and Intravaginal Challenge

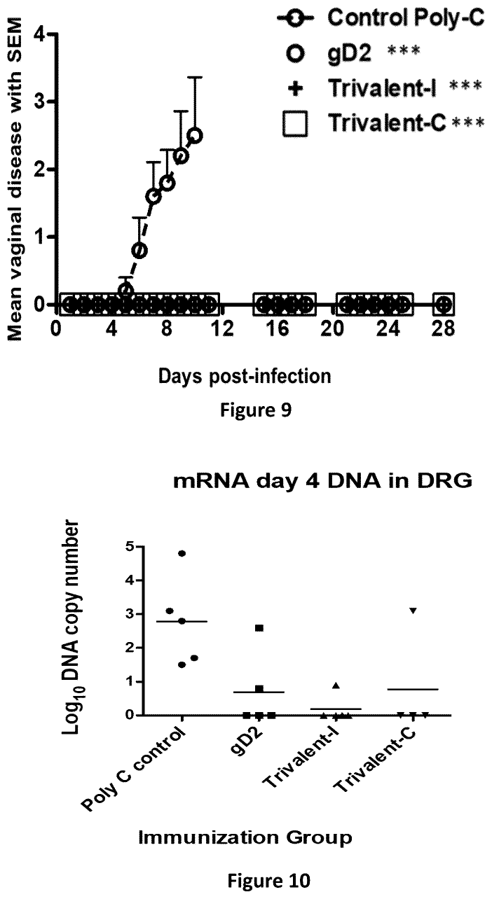

Animals were monitored daily for genital disease for 28 days post challenge. A score of 0 was assigned for no disease, and 1 point each was assigned for hair loss around the anal or genital orifices, genital erythema, genital exudate, and necrosis of genital tissues (FIG. 9). No animal in the gD2 or trivalent RNA/LNP groups developed genital disease, which was significantly different than the poly C controls (p<0.001, one-way ANOVA by Kruskal-Wallis test followed by Dunn's multiple comparisons for significance).

### Example 10: HSV-2 DNA in Dorsal Root Ganglia after Modified RNA Immunization and Intravaginal Challenge

Five animals per group were euthanized at 4 days post challenge, except for the trivalent-combined group in which four animals were euthanized. Dorsal root ganglia (DRG) were harvested for HSV-2 DNA quantitation by qPCR to detect the Us9 gene. All five animals in the poly C group had HSV-2 DNA detected in the DRG, while ⅖ animals in the gD2 group, ⅕ in the trivalent RNA administered at individual sites group (Trivalent-I), and ¼ in the trivalent modified RNA given at the same site group (Trivalent-C) were positive for HSV-2 DNA (FIG. 10; Mann-Whitney test: gD2 group compared with poly C, p=0.03; trivalent at different sites compared with poly C, p<0.01; Trivalent-I compared with poly C, p=0.14). The difference between the modified RNA immunized groups was not significant.

Summary: Dorsal root ganglia were negative for HSV-2 DNA on day 4 after infection in 75% to 80% of animals immunized with modified RNA encoding gD2 immunogenic fragment alone or the trivalent vaccine. The trivalent RNA group administered individually at different sites and the gD2 groups significantly outperformed the poly C RNA control group, while the trivalent RNA group administered together did not differ significantly from the poly C group, likely because of the smaller sample size in the trivalent-combined group.

Modified RNA vaccines expressing gD2 immunogenic fragment alone or gC2, gD2 and gE2 (trivalent) immunogenic fragments provided outstanding protection against HSV-2 genital challenge. The expression of the three exemplary immunogenic fragments slightly outperformed exemplary gD2 immunogenic fragment based on day 2 and day 4 titers after challenge and the lower number of animals with HSV-2 DNA detected in DRG on day 4.

### Example 11: T Follicular Helper (Tfh) Cell and Germinal Center B Cell Responses in Immunized Mice

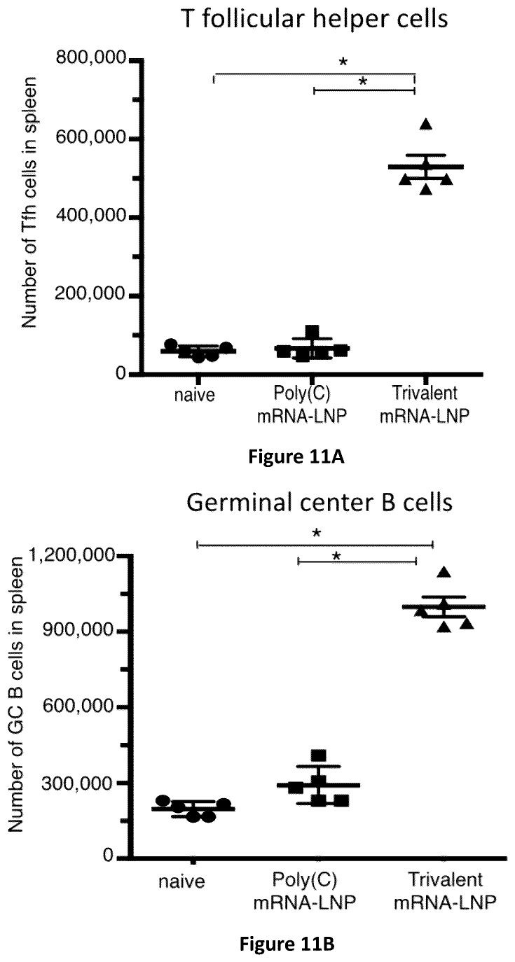

BALB/c female mice were left un-immunized as naïve control animals or immunized intradermally twice at 28 day intervals with poly C RNA/LNP or trivalent modified RNA-LNP. The poly C RNA controls received 10 μg Poly C RNA-LNP divided into 4 aliquots and administered at 4 separate sites. The trivalent modified RNA group received 10 μg RNA encoding exemplary gC2 immunogenic fragment/LNP, 10 μg modified RNA encoding exemplary gD2 immunogenic fragment/LNP, and 10 μg modified RNA encoding exemplary gE2 immunogenic fragment/LNP each divided into 2 aliquots and each given at 2 sites. Two weeks after the second immunization, spleens were harvested from 5 animals per group and flow cytometry performed to detect T follicular helper (Tfh) cells (FIG. 11A; *p<0.05) and germinal center B cell responses (FIG. 11B; *p<0.05).

Conclusion: The trivalent modified RNA/LNP vaccine induced a potent Tfh and germinal center B cell response and significantly outperformed the poly C control immunization (p<0.05) and the naïve group (p<0.05) for both Tfh and germinal center B cell responses. These immune responses suggest that the trivalent modified RNA/LNP vaccine will likely induce a durable antibody response.

### Example 12: Vaginal IgG Responses to Modified RNA Immunization in Mice

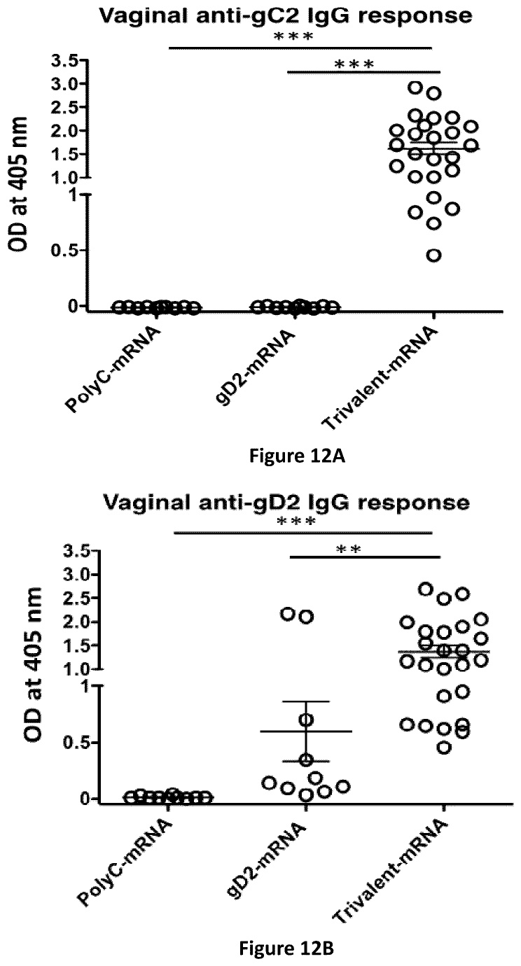

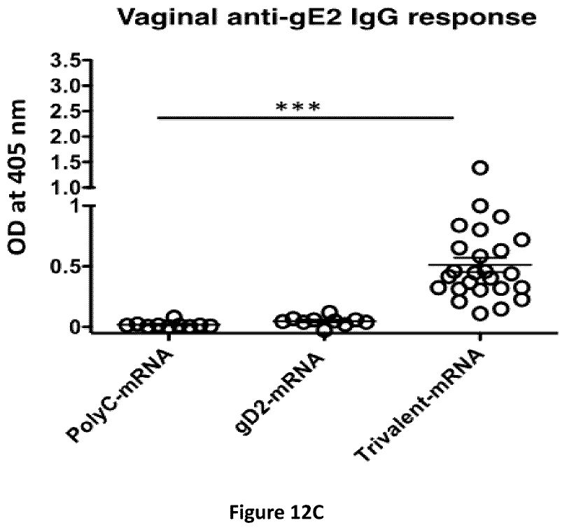

BALB/c mice were immunized intradermally twice at 28 day intervals with 10 μg of poly C RNA-LNP, 10 μg modified RNA encoding exemplary gD2 immunogenic fragment/LNP or 10 μg trivalent modified RNA encoding each of exemplary gC2, gD2, and gE2 immunogenic fragments/LNP. The trivalent modified RNA was combined and administered as 10 μg modified RNA encoding exemplary gC2 immunogenic fragment, 10 μg modified RNA encoding exemplary gD2 immunogenic fragment, and 10 μg modified RNA encoding exemplary gE2 immunogenic fragment combined into LNP and divided into 4 aliquots and given at 4 sites. One month after the second immunization, 60 μl of media were introduced in the vaginal cavity and retrieved. IgG titers to gC2 (FIG. 12A), gD2 (FIG. 12B), and gE2 (FIG. 12C) were determined at a 1:50 dilution of the vaginal wash fluids by ELISA (FIGS. 12A-12C, n=10 mice in the poly C group, n=10 in the gD2 modified mRNA group and n=25 in the trivalent modified mRNA group; ***p<0.001; **p<0.01).

Conclusion: The trivalent modified RNA produced a robust vaginal IgG response to gC2 (FIG. 12A) and gD2 (FIG. 12B) and a more moderate response to gE2 (FIG. 12C). The gD2 ELISA titers were higher in mice immunized with the trivalent RNA vaccine compared to mice immunized with the gD2 RNA vaccine (FIG. 12B).

### Example 13: Antibodies to Exemplary gC2 Immunogenic Fragment Produced by Trivalent RNA Immunization of Mice Block Immune Evasion Domains on gC2

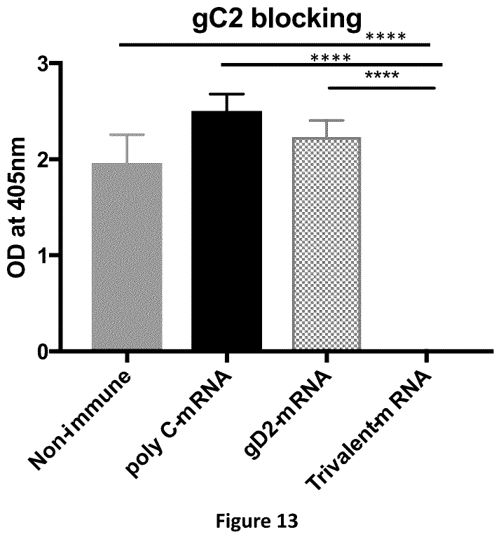

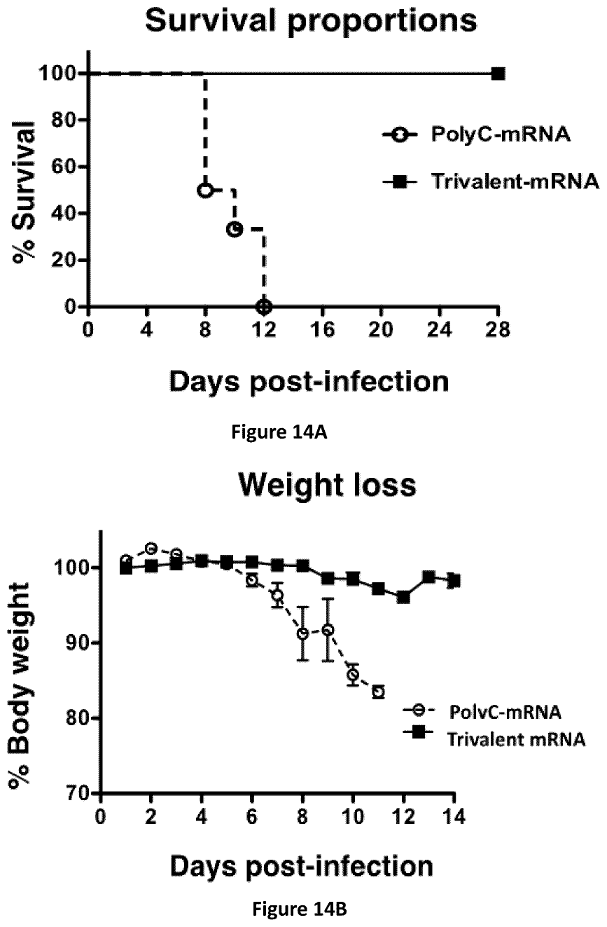

BALB/c mice were left unimmunized as a source of non-immune IgG, or immunized intradermally with poly C RNA/LNP or trivalent modified RNA/LNP. The poly C RNA controls received 10 μg poly C RNA/LNP divided into 4 aliquots and administered at 4 separate sites. The gD2 group received 10 μg modified RNA encoding exemplary gD2 immunogenic fragment/LNP administered as described for the poly C RNA/LNP. The trivalent modified RNA group received 10 μg modified RNA encoding exemplary gC2 immunogenic fragment/LNP, 10 μg modified RNA encoding exemplary gD2 immunogenic fragment/LNP, and 10 μg modified RNA encoding exemplary gE2 immunogenic fragment/LNP combined into one LNP and divided into 4 aliquots and given at 4 sites. There were 10 mice in each group. Sera from the 10 mice were pooled and IgG was purified. The IgG was evaluated at 12 μg/200 μl for its ability to block complement component C3b binding to gC2. This blocking assay is used to assess whether antibodies produced by immunization block the immune evasion properties of gC2. Non-immune murine IgG, IgG from the poly C RNA group, and IgG from the gD2 group each failed to block gC2 binding to C3b. In contrast, IgG from trivalent modified RNA-immunized animals totally blocked the interaction between gC2 and C3b (FIG. 13, ****p<0.0001).

Conclusions: The trivalent modified RNA vaccine produces antibodies that block immune evasion domains on gC2 as determined by blocking the interaction between gC2 and C3b.

### Example 14: Intravaginal Infection of Mice at a Higher Inoculum Titer of HSV-2 after Modified RNA Vaccination

BALB/c mice (n=5) were immunized with the trivalent modified RNA using 10 μg modified RNA encoding exemplary gC2 immunogenic fragment/LNP, 10 μg modified RNA encoding exemplary gD2 immunogenic fragment/LNP, 10 μg modified RNA encoding exemplary gE2 immunogenic fragment/LNP each divided into 2 aliquots and each given individually at 2 sites. One month after the second immunization, mice were treated with medroxyprogesterone and five days later infected intravaginally with 5×10⁴ PFU HSV-2 strain MS (2,000 LD₅₀). Animals were followed for 28 days and evaluated for death, genital disease, vaginal viral titers 2 and 4 days after infection and dorsal root ganglia (DRG) HSV-2 DNA copy number 28 days after infection. No mouse immunized with the trivalent modified RNA/LNP vaccine died, had genital disease, had any virus detected on day 2 or 4 post-infection or had HSV-2 DNA detected in DRG (Table 1).

TABLE 1
Trivalent modified RNA/LNP immunized mice challenged
with HSV-2 strain MS (2,000 LD₅₀)

| Disease parameters | Mice | % Protection |
|---|---|---|
| Death | 0/5 | 100 |
| Genital disease | 0/5 | 100 |
| Vaginal viral titers day 2 | 0/5 | 100 |
| Vaginal viral titers day 4 | 0/5 | 100 |
| HSV-2 DNA copies in DRG | 0/5 | 100 |

Conclusions: Mice were infected with HSV-2 at a dose that was 10-fold higher than used in earlier experiments described herein (FIGS. 7A-10). Protection of the mice remained outstanding even at this higher titer challenge. All five mice achieved sterilizing immunity as determined by no deaths, no genital disease, negative vaginal virus titers on days 2 and 4 post-infection and no HSV-2 DNA in the lumbosacral DRG on day 28 (Table 1).

### Example 15: Evaluation of the Intramuscular Route of Modified RNA Immunization in Mice

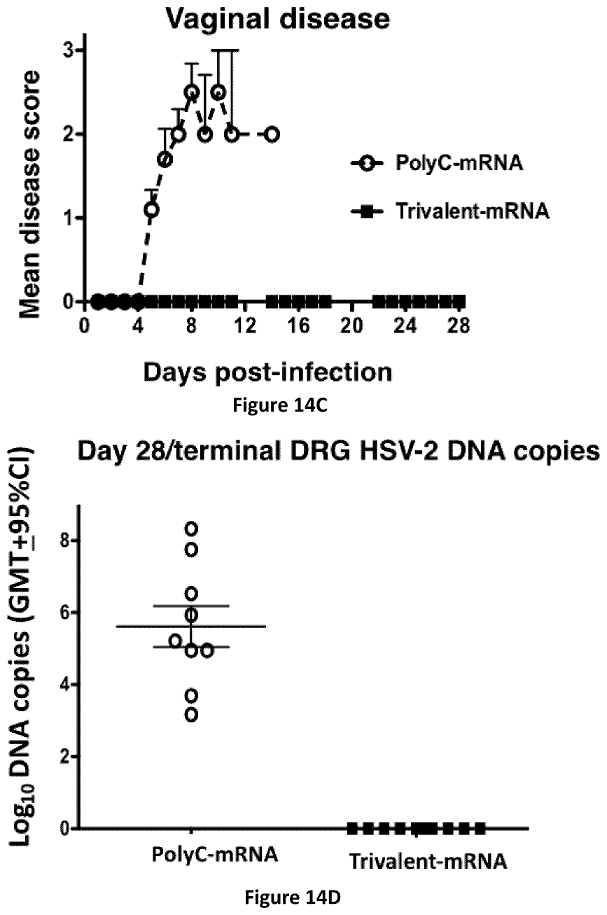

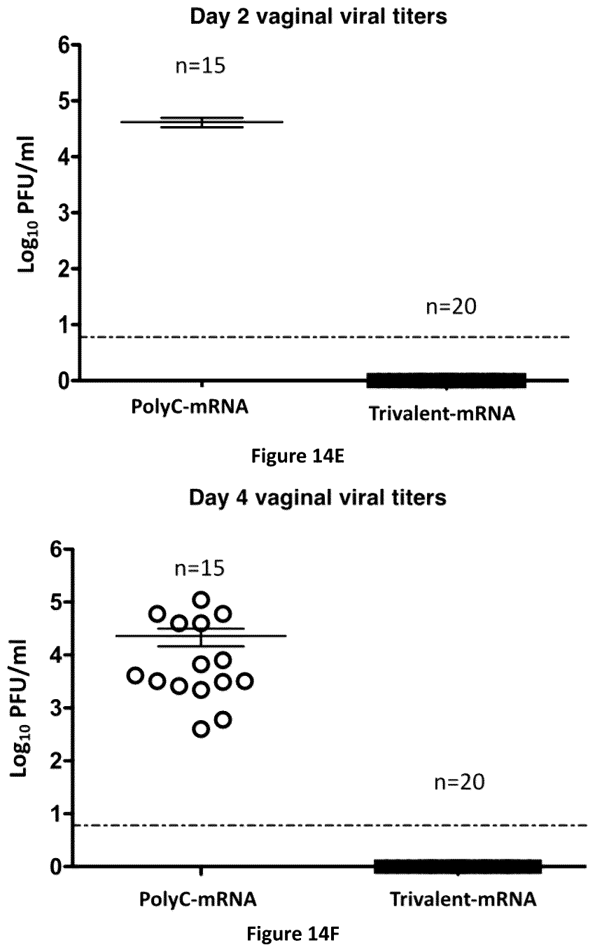

BALB/c mice were immunized intramuscularly with poly C RNA/LNP as a control (15/group) or with trivalent modified RNA containing 10 μg of modified RNA encoding each of exemplary gC2, gD2, and gE2 immunogenic fragments/LNP (20/group). All poly C control animals died by day 12, while all animals in the trivalent modified RNA group survived (FIG. 14A). No weight loss occurred in the trivalent modified RNA group, while the poly C control animals lost >15% of body weight (FIG. 14B). The poly C group developed extensive genital disease, while the trivalent modified RNA animals had no genital disease (FIG. 14C). DRG were harvested from nine poly C animals at the time of euthanasia between days 7 and 12 post-infection or at the end of the experiment on day 28 in the trivalent modified RNA group. All animals in the poly C group had HSV-2 DNA detected in DRG, while none were positive for HSV-2 DNA in trivalent modified RNA group (FIG. 14D). Day 2 (FIG. 14E) and Day 4 (FIG. 14F) vaginal viral cultures were positive in all 15 animals in the poly C group, while cultures were negative in all 20 animals in the trivalent modified RNA group. Differences between poly C and trivalent groups are significant, p<0.001 for each figure (FIGS. 14A-14F).

Conclusions: Trivalent modified RNA/LNP provides outstanding protection in mice when administered intramuscularly. Comparable findings to the above were observed when mice were immunized intradermally. Overall, 64 mice have been evaluated, which were immunized with trivalent modified RNA at 10 μg of each immunogenic fragment given either intradermally (FIGS. 7A-10) or intramuscularly (FIGS. 14A-14F). Sterilizing immunity was achieved in 63/64 (98%) mice based on no death, no genital disease, no weight loss, negative day 2 and 4 vaginal titers and negative HSV-2 DNA in DRG.

### Example 16: Summary Comparison of Immunization with Trivalent RNA/LNP and Trivalent Subunit Antigen CPG/ALUM in BALB/c Mice

The results presented in Table 2 hereinbelow represent a summary of all the results in BALB/c mice that were immunized either intradermally or intramuscularly with trivalent modified RNA containing 10 μg of modified RNA encoding gC2, gD2 and gE2 immunogenic fragments/LNP (total 64 mice studied). A comparison is shown with the results obtained in BALB/c mice that were immunized with 5p g each of bac-gC2(27-426t) containing gC2 amino acids 27-426 from HSV-2 strain 333, bac-gD2(306t) containing gD2 amino acids 26-331 from HSV-1 strain 333, and bac-gE2(24-405t) containing gE2 amino acids 24-405 from HSV-2 strain 2.12. The exemplary gC2, gD2, gE2 fragment antigens were mixed with 150 μg CpG and 25 μg alum/per μg protein as adjuvants and administered intramuscularly. Mice were immunized twice at 28-day intervals with trivalent modified RNA/LNP and three times at 14-day intervals with exemplary gC2, gD2, gE2 fragment antigens, as done in prior experiments. The modified RNA and gC2, gD2, gE2 fragment antigen experiments were performed at the same time. The results summarized in Table 2 demonstrate significant superiority of the trivalent modified RNA/LNP vaccine over the trivalent gC2, gD2, gE2 fragment antigen vaccine in many immune response parameters, and most importantly in vaccine efficacy. The trivalent modified RNA/LNP vaccine achieved sterilizing immunity in 63/64 (98%) of mice compared to 15/20 (75%) in the subunit antigen group.

TABLE 2
Comparisons of immunization with trivalent modified RNA/LNP
or trivalent exemplary gC2,gD2,gE2 fragment antigen
CpG/alum in BALB/c mice.

| | | Trivalent | | |
|---|---|---|---|---|
| | | exemplary | | |
| | | gC2, gD2, | | |
| | Trivalent | gE2 fragment | | |
| Comparison | modified RNA | antigen | | P value |
| Serum IgG ELISA | | | | |
| gC2 | 1:256,000 | 1:32,000 | | p < 0.001 |
| gD2 | 1:512,000 | 1:128,000 | | p < 0.01 |
| gE2 | 1:64,000 | 1:16,000 | | p < 0.05 |
| Vaginal fluid IgG | | | | |
| ELISA OD at 405 | | | | |
| nm tested at | | | | |
| 1:50 dilution | | | | |
| gC2 | 1.6 OD | 0.6 | OD | p < 0.001 |
| gD2 | 1.5 OD | 1.0 | OD | p < 0.05 |
| gE2 | 0.5 OD | 0.25 | OD | p < 0.01 |
| Serum neutralizing | | | | |
| antibody | | | | |
| Against HSV-2 | 1:4,800 | 1:1,600 | | p < 0.01 |
| Against HSV-1 | 1:6,400 | 1:4,000 | | p = NS* |
| Blocking C3b | Total | Total | | p = NS |
| binding to gC2 | blocking | blocking | | |
| CD4⁺ T cell | Significant | Significant | | Trivalent |
| responses | responses for | response | | modified |
| | gC2, gD2 | only for gD2 | | RNA more |
| | and gE2 | | | potent |
| CD8⁺ T cell | Significant | No significant | | Trivalent |
| responses | response for | responses | | modified |
| | gE2 | | | RNA more |
| | | | | potent |

| | | | |
|---|---|---|---|
| Achieving | 63/64 (98%) | 15/20 (75%) | p < 0.01 |
| sterilizing | mice | mice | |
| immunity^{#} | | | |

*NS, not significant;
^{#}Sterilizing immunity defined as no death, no genital disease, no weight loss, and no evidence of subclinical infection as measured by day 2 and day 4 vaginal cultures post-infection and HSV-2 DNA in dorsal root ganglia on day 4 or day 28 post-infection.

### Example 17: Evaluation of the Trivalent Modified RNA/LNP Vaccine in Guinea Pigs

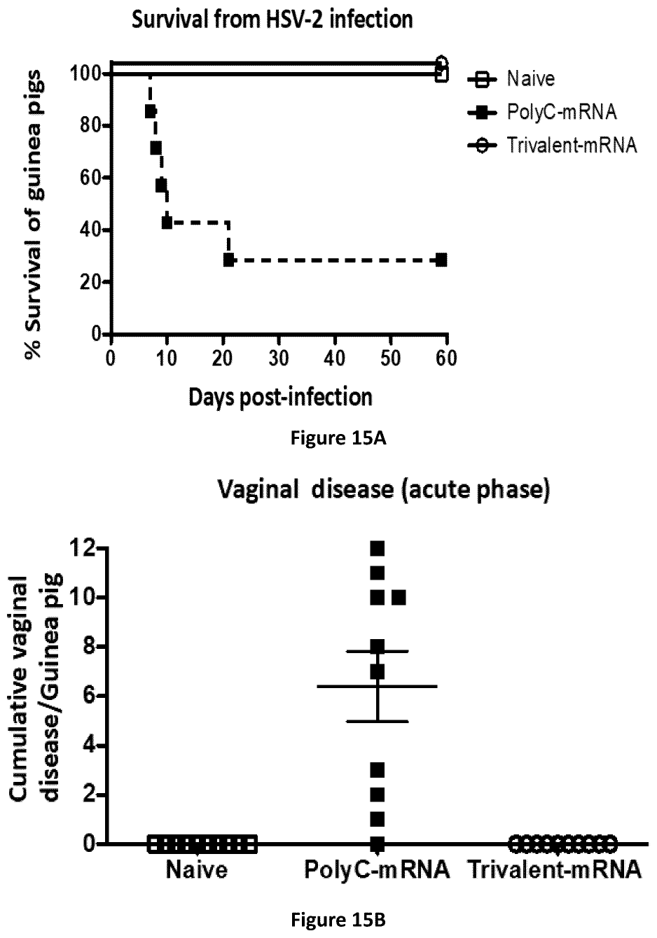

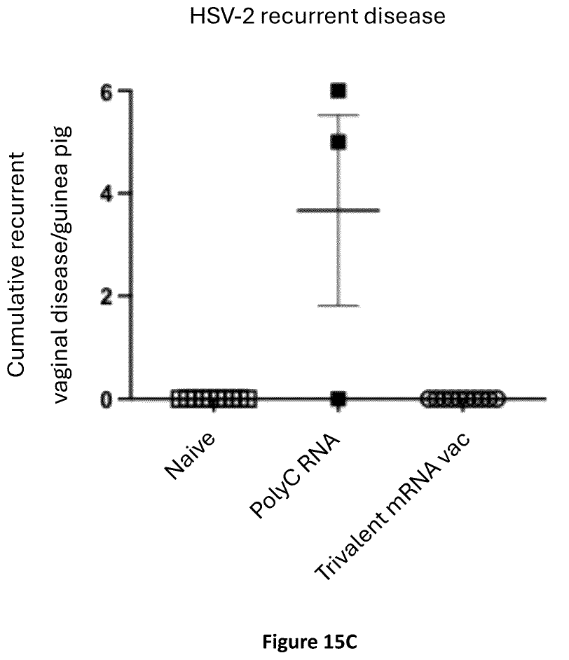

Hartley Strain female guinea pigs were left unimmunized and uninfected (naive group, n=10), immunized three times intradermally at monthly intervals with 20 μg poly C RNA-LNP (n=10) or with 20 μg of modified RNA encoding each of exemplary gC2, gD2, gE2 immunogenic fragments/LNP (n=10). One month after the final immunization, animals in the poly C and trivalent modified RNA groups were infected intravaginally with 5×10⁵ PFU of HSV-2 strain MS (50 LD₅₀). Animals were observed for death, genital lesions during the acute phase of infection (days 1-14) and genital lesions during the recurrent phase of infection (days 15-60). In the poly C control group, 7/10 animals died or were humanely euthanized between days 7 and 20 post-infection, while no animal in the trivalent group and no naïve (uninfected) animal died (FIG. 15A). The poly C group had genital lesions on a mean of 6.4 days during the acute phase of infection with 9/10 animals developing acute genital disease, while no animal in the trivalent group or naïve (uninfected) group developed acute genital disease (FIG. 15B). The poly C animals had genital lesions on a mean of 3.7 days during the recurrent phase of infection from days 15-60, with ⅔ animals developing recurrent genital lesions (FIG. 15C). In contrast, the trivalent immunized guinea pigs and the naïve (uninfected) animals had no recurrent genital lesions (FIG. 15C).

Conclusions: Trivalent modified RNA/LNP provided outstanding protection against acute and recurrent genital lesions in guinea pigs.

### Example 18: Evaluation of the HSV-1 and HSV-2 Trivalent Nucleoside-Modified RNA/LNP Vaccines Against Genital and Non-Genital HSV-1 Infection in Mice

The present Example demonstrates that an RNA vaccine encoding exemplary HSV gC, gD, and gE immunogenic fragments is effective against genital HSV-1 and HSV-2 infection, as well as HSV-1 oral infection. Further, the present Example demonstrates the unexpected finding that an RNA vaccine encoding HSV-2 gC, gD, and gE (“tri-HSV-2”) showed similar efficacy against HSV-1 as an RNA vaccine encoding HSV-1 gC, gD, and gE (“tri-HSV-1”).

### Materials and Methods

Tri-HSV-1 and tri-HSV-2 modified RNA encoding exemplary immunogens. The tri-HSV-2 immunogens were prepared to generate RNA that encodes exemplary immunogenic fragments of the ectodomains of HSV-2 gC (27-426; SEQ ID NO: 11), HSV-2 gD (26-333; SEQ ID NO: 25), and HSV-2 gE (24-405; SEQ ID NO: 17). The tri-HSV-1 exemplary immunogens were prepared to generate RNA that encodes exemplary immunogenic fragments of the ectodomains of the HSV-1 gC (27-457; SEQ ID NO: 8), HSV-1 gD (26-333; SEQ ID NO: 26), and HSV-1 gE (24-309; SEQ ID NO: 27) proteins where the first amino acid listed represents the amino acid immediately after the signal sequence. Each subunit protein extends from the first amino acid after the signal sequence to just prior to the transmembrane domain.

To generate nucleoside-modified RNA, m1Ψ-5′-triphosphate (TriLink) was used instead of UTP. RNA was capped using the m7G capping kit with 2′-O-methyltransferase (ScriptCap, CellScript). The RNA contains a 101-nucleotide poly(A) tail. Nucleoside-modified RNAs were purified by Fast Protein Liquid Chromatography (FPLC) (Akta Purifier GE Healthcare) and stored at −20° C. FPLC nucleoside-modified RNAs and polyC RNA (Sigma) were encapsulated in LNPs using a self-assembly process in which an aqueous solution of RNA at acidic pH 4.0 was rapidly mixed with a solution of lipids dissolved in ethanol. LNPs used in this study were similar in composition to those described previously, which contain an ionizable cationic lipid/phosphatidylcholine/cholesterol/PEG-lipid (Acuitas) (50:10:38.5:1.5 mol/mol) and were encapsulated at an RNA to total lipid ratio of ˜0.05 (wt/wt). The LNPs had a diameter of ˜80 nm as measured by dynamic light scattering using a Zetasizer Nano ZS (Malvern Instruments Ltd, Malvern, UK) instrument, and were stored at −80° C. at a concentration of RNA of ˜1 μg/μl.

The three modified RNAs encoding exemplary HSV-1 immunogenic fragments were combined in equal concentrations based on mass prior to encapsulation in lipid nanoparticles by Acuitas. The vaccine formulations were stored at −80° C. and not reused once thawed.

Immunization of mice. Female BALB/c mice aged 6-8 weeks old were immunized twice 30 days apart in the hind right gastrocnemius muscle. Mice were immunized with either 1 μg or 10 μg (total) of tri-HSV-1 or tri-HSV-2 modified RNA vaccines. Control mice were injected with an equal volume of sterile saline. Serum was collected 25 days after the final immunization and stored at −80° C.

Serum IgG ELISA endpoint titers. Purified baculovirus-derived exemplary gC1, gD1, and gE1 immunogenic fragments were produced as previously described. Purified protein was added to High Binding Costar microtiter plates. Serial 2-fold dilutions of serum starting at 1:250 were added to protein coated wells. Anti-mouse IgG was detected by adding 2,2′-azino-bis-3-ethylbenzothiazoline-6-sulfonic acid (ABTS). Endpoint titers were calculated as the serum concentration that had an absorbance value of ≥0.1 and was ≥two-fold higher than serum from PBS immunized mice.

HSV-1 and HSV-2 neutralizing antibody titers. Neutralizing titers were measured by incubating 2-fold serial dilutions of mouse sera with 100 PFU of HSV-1 strain NS at 37° C. for 1 hour. Incubations were done in the presence of 5% human serum as a source of complement obtained from an HSV-1/HSV-2 seronegative donor. Remaining infectious virus was determined by plaque assay on Vero cells. The neutralizing titer was defined as the highest serum dilution that reduced plaques by ≥50%.

Isolation and stimulation of splenocytes from BALB/c mice. Mice were immunized twice with PBS or 10 μg of exemplary tri-HSV-1 or tri-HSV-2 vaccine. Immunized mice were sacrificed 10 days after the second immunization. The spleens were harvested, and mechanically dissociated into single-cell suspensions. Cell counts were obtained and 2×10⁶ cells were added to individual wells for each stimulation condition for each mouse. Splenocytes were stimulated in RPMI media supplemented with 55 μM 2-mercaptoethanol and 10 units recombinant mouse IL-2/μL stimulation media. Intracellular cytokines were immobilized with 5 μg/mL Brefeldin A and Monensin. Cells were stimulated with Dimethyl Sulfoxide (DMSO), 0.04 μM phorbol-12-myristate 13-acetate (PMA) with 0.67 μM ionomycin, or 1 μg/mL of HSV-1 protein overlapping peptide pools. Cells were incubated for 12 hours at 37° C. in 5% CO₂.

Overlapping peptide pools. Overlapping peptide pools were synthesized by GenScript with a purity of ≥70%. Peptides were 15 amino acids long with an 11 amino acid overlap. Identical amino acid sequences used in the modified RNA encoding exemplary immunogenic fragments were used for synthesizing overlapping peptide sequences. All peptide pools were resuspended in DMSO and RPMI to a concentration of 2 μg/μL and stored at −80° C.

Flow cytometry reagents. All antibodies were purchased from BioLegend. Antibodies used for labeling cells were fluorescein isothiocyanate (FITC) conjugated anti-CD3 (clone 17A2), allophycocyanin (APC)-Fire 750 conjugated to CD4 (Clone GK1.5), phycoerythrin (PE) conjugated anti-IL-2 (cloneJES6-5H4), PE-Dazzle 594 conjugated anti-CD107a (clone 1D4B), Peridinin-Chlorophyll-Protein-Cyanin (PerCP-Cy5.5) conjugated anti-CD8α (clone 53-6.7), Alexa-Flour (AF)-700 conjugated anti-IFNγ (clone XMG1.2), Brilliant Violet 421 conjugated anti-IL-4 (Clone 11b11) and PE-Cy7 conjugated anti-TNFα (clone MP6-XT22). Dead cells were discriminated by Fixable Live-Dead Aqua dye (Invitrogen). Brefeldin A, PMA/ionomycin, Cyto-fast-fix-perm buffer, and intracellular staining permeabilization wash buffer were purchased from BioLegend. After labeling of intracellular cytokines, cells were fixed with 1% formaldehyde in PBS and stored at 4° C. until the next day. Cells were analyzed on a BD LSR 2. Gates were set to discriminate singlet cells and then lymphocytes based on forward scatter and side scatter properties. 150,000 events in the lymphocyte gate were collected. After data collection, flow samples were analyzed using FlowJo version 10.8 (Tree Star software). Cells were gated on CD4+ or CD8+ T-cells producing single cytokines. Boolean combination gates were used to compare IL-2, TNFα, and IFNγ production.

HSV-1 lip infection. Mice were challenged 30 days after the 2^{nd} immunization. Mice were anesthetized with a solution of 100 mg/kg Ketamine and 12.5 mg/kg Xylazine, placed on their back, and a 25G needle was used to make 10 vertical scratches on the lower lip. After scratching the lip, 2×10⁶ PFU of HSV-1 strain NS was topically applied. Mice were monitored for 13 days for weight loss and presence of lesions on the lower lip.

Lip and Trigeminal (TG) tissue isolation. The lip and TG were removed and placed in tubes containing 500 μL DMEM supplemented with 5% FBS. The tissues were stored at −80° C. until further processing. Lip and TG tissues were subjected to 3 freeze-thaw cycles. The lip was homogenized in a glass grinder and resuspended in 1 mL of media. The TG was homogenized using the frosted ends of glass slides and resuspended in 275 μL of DMEM. Tissue homogenates were used to make 10-fold serial dilutions that were then evaluated by plaque assay on Vero cells.

HSV-1 vaginal infection. Mice were challenged with 2×10⁶ PFU of HSV-1 strain NS intravaginally. Mice were scored daily for 14 days on a basis of one point each for redness/erythema, hair loss, distended belly indicating loss of fecal motility, and urinary staining for a maximum daily score of four. The vaginal canals of mice were swabbed on days 2 and 4 for virus titers. Vaginal swabs were placed in 1 mL of DMEM supplemented with L-glutamine, HEPES, antibiotics, and 5% FBS. Serial 10-fold dilutions were evaluated by plaque assay on Vero cells.

HSV-1 DNA isolation. The DRG were harvested at the time of sacrifice of the mice either at the end of the experiment on day 28 post-challenge or prior to day 28 in mice in which humane euthanasia was required. Purified DNA was isolated and amplified for qPCR as previously described and the HSV-1 DNA copy number was expressed as log₁₀ DNA copies per 10⁵ adipsin genes. Samples with less than one copy by 40 cycles in duplicate wells were considered negative. If only one of the duplicate wells was positive, the sample was tested in triplicate and considered positive if two or more of the triplicates were positive.

Statistics. The log-rank test was used to calculate P values for survival curves. The Mann-Whitney test was used to calculate P values in experiments evaluating vaginal titers, serum antibodies by ELISA, HSV-1 neutralizing titers, viral titers present in lip and TG tissue, and HSV-1 DNA copy number in the lip, TG, and DRG. The flow cytometry data was analyzed using 2-way ANOVA with correction for multiple comparisons. All significance tests were two-tailed with a P value <0.05 considered significant. Analysis was done using GraphPad Prism version 9.5 (GraphPad software Inc).

### Results

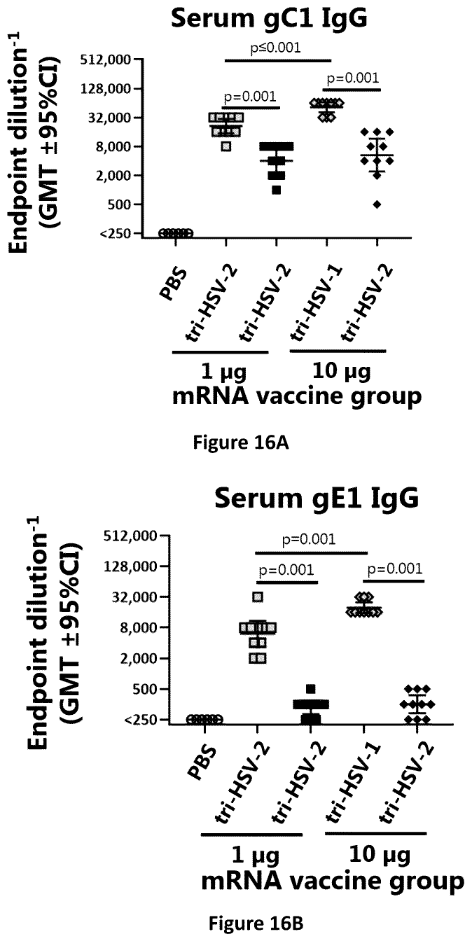

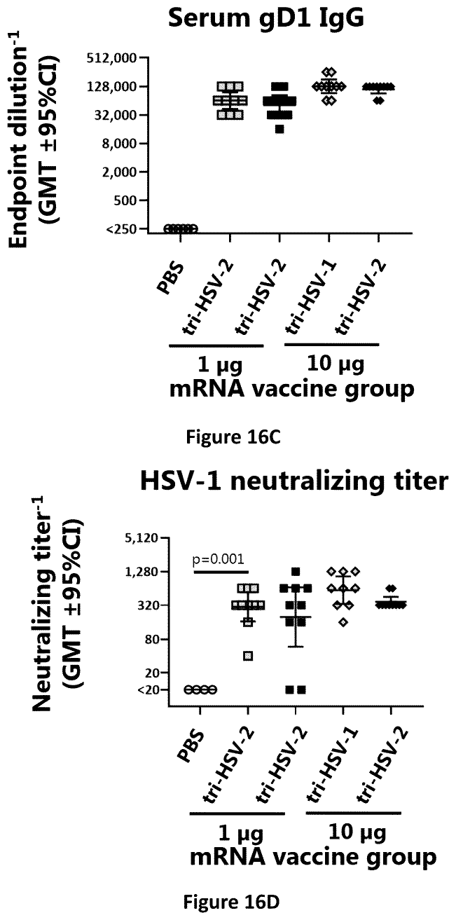

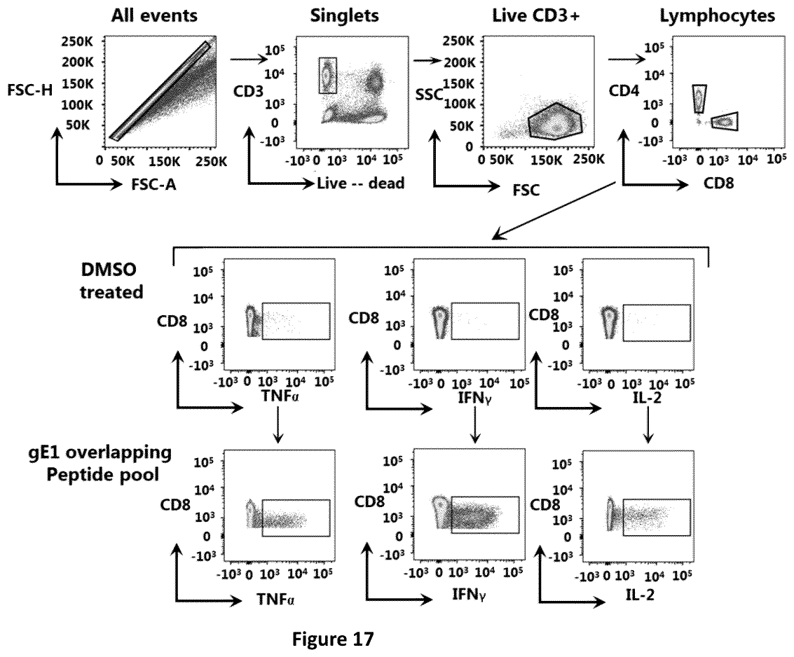

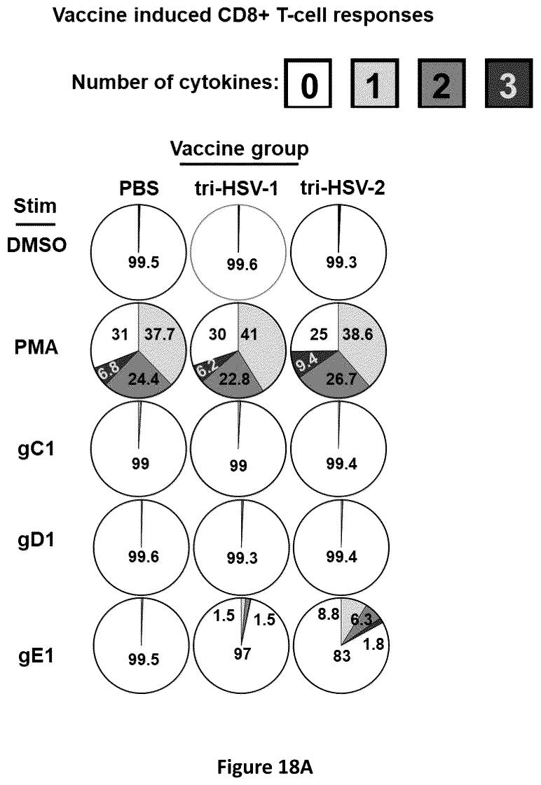

The efficacy of exemplary tri-HSV-1 vaccine and exemplary tri-HSV-2 vaccine against HSV-1 challenge to lip or vagina was compared. To better resolve differences between the vaccines, mice were immunized with either a 1 μg (low) or 10 μg (high) dose of total modified RNA containing equal concentrations of each modified RNA immunogen. The tri-HSV-1 immunized mice had significantly higher serum IgG ELISA antibodies to gC1 and gE1 than tri-HSV-2 immunized animals (FIGS. 16A-16B). The gC1 IgG levels were 4.6-fold higher in the tri-HSV-1 group than in the tri-HSV-2 group at 1 μg and 6.9-fold higher at 10 μg doses. A dose response with the tri-HSV-1 vaccine was detected in that the gC1 IgG levels were 2.3-fold higher in animals immunized with 10 μg than 1 μg, while no differences were detected at the two doses in the tri-HSV-2 immunized mice (FIG. 16A). The gE1 IgG levels were 35- and 72-fold higher in tri-HSV-1 than tri-HSV-2 immunized mice at the low and high doses, respectively. A 2.5-fold dose-response increase in gE1 IgG titers in the high dose tri-HSV-1 immunized mice (FIG. 16B) was detected. The gD1 IgG levels were not significantly different comparing tri-HSV-1 and tri-HSV-2 immunized mice at the low or high dose (FIG. 17C). The IgG ELISA results suggest that many epitopes on gD1 and gD2 are type common, while epitopes on gC1 and gC2, or gE1 and gE2 are type specific.

Sera was evaluated for HSV-1 neutralizing antibodies in the presence of 5% human serum as a source of complement. Neutralizing titers were higher in the tri-HSV-1 vaccine immunized mice at both immunization doses, but differences did not reach statistical significance (FIG. 16D). The neutralizing antibody results are consistent with the gD ELISA results showing comparable IgG binding titers to gD1 produced by the tri-HSV-1 and tri-HSV-2 vaccines. The neutralizing titers are also consistent with the prior observation that gD antibodies are more potent at neutralizing virus than gC or gE antibodies.

Tri-HSV-2 immunization induces a more robust CD8+ T-cell responses to gE1 overlapping peptides than tri-HSV-1 immunization.

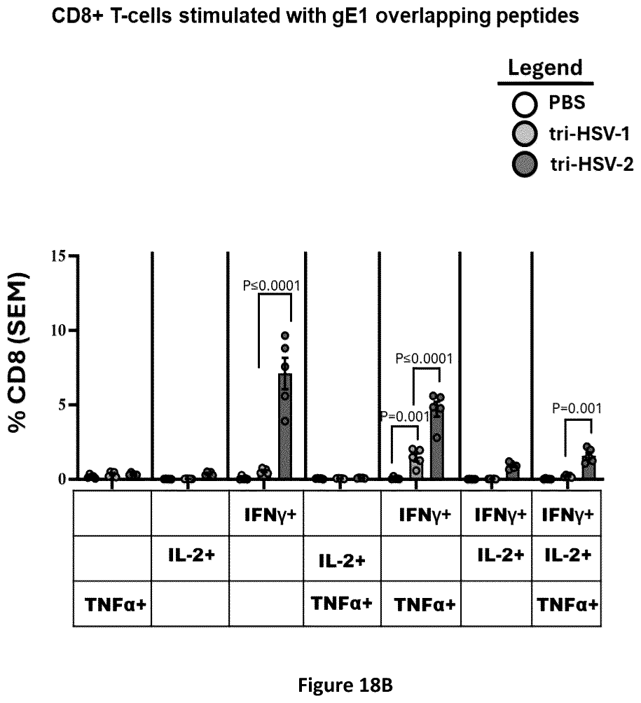

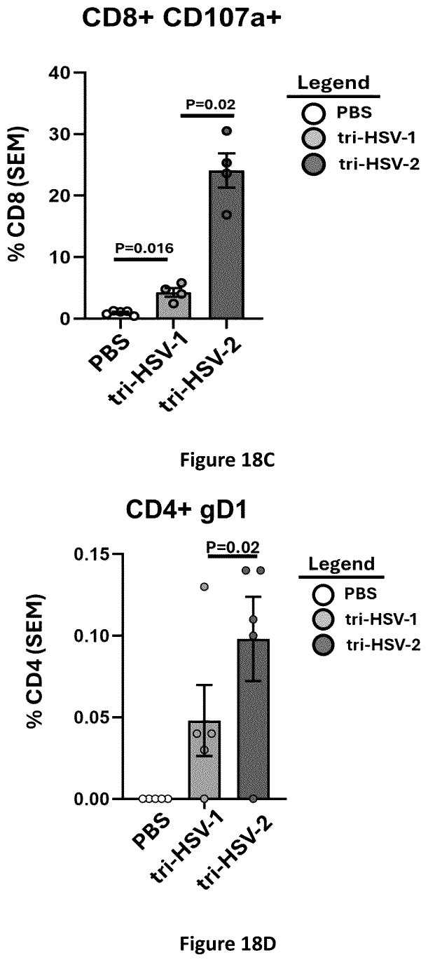

The T-cell response in mice immunized twice with 10 μg tri-HSV-1 or tri-HSV-2 vaccines was evaluated. Splenocytes were stimulated with overlapping peptide pools of gC1, gD1, or gE1. Responding cells were measured by intracellular cytokine flow cytometry (FIG. 17). CD8+ T cell responses to gC1 or gD1 in tri-HSV-1 or tri-HSV-2 immunized mice were not detected (FIG. 18A). However, robust CD8+ T cell responses to gE1 overlapping peptides was detected, particularly in mice immunized with the tri-HSV-2 vaccine (FIG. 18A). The number of IFNγTNFα⁺ (double positive) CD8+ T-cells was 3.4-fold higher in the tri-HSV-2 immunized mice than the tri-HSV-1 immunized vaccine group (FIG. 18B, 3^{rd} group from right). Additionally, the number of triple positive CD8+ T-cells (IFNγ⁺TNFα⁺IL-2⁺) was 8.2-fold higher in the tri-HSV-2 vaccine immunized group compared to the tri-HSV-1 vaccine immunized group (FIG. 18B, last group on right). CD8+ T-cell activity was analyzed by CD107a labeling, a marker of degranulation. The tri-HSV-1 vaccine immunized group had more activated T-cells compared to the PBS group, but the tri-HSV-2 vaccine immunized animals had 7-fold greater CD107a labeling compared to the tri-HSV-1 vaccine immunized animals (FIG. 18C). CD4+ T-cells stimulated with gD1 overlapping peptides were analyzed. CD4+ T-cells in the tri-HSV-2 vaccine immunized group had a 2-fold increase in polyfunctional cells (IFNγ⁺TNFα⁺IL-2⁺) compared to tri-HSV-1 vaccine immunized mice (FIG. 18D). Responding cells were evaluated to determine whether the cells have a Th1 or Th2 phenotype by calculating the ratio of IFNγ+(Th1) to IL-4+ (Th2) responses. A high ratio of IFNγ to IL-4 was observed, indicating a Th1 phenotype (Table 3).

TABLE 3
Ratio of CD4+ T-cells producing IFNγ to IL-4 following
stimulation with gD1 overlapping peptides.

| | |
|---|---|
| PBS | 1.25 |
| Tri-HSV-1 | 8.52 |
| Tri-HSV-2 | 10.16 |

CD4+ T cell responses to gC1 or gE1 overlapping peptides in exemplary tri-HSV-1 or exemplary tri-HSV-2 vaccine immunized mice were not detected. These results indicate that immunization with exemplary tri-HSV-2 RNA vaccine induces CD4+ and CD8+ effector T-cell responses in mice to peptides from HSV-1, and that some CD4+ and CD8+ T cell responses to the exemplary tri-HSV-2 vaccine are even more potent than to the exemplary tri-HSV-1 vaccine.

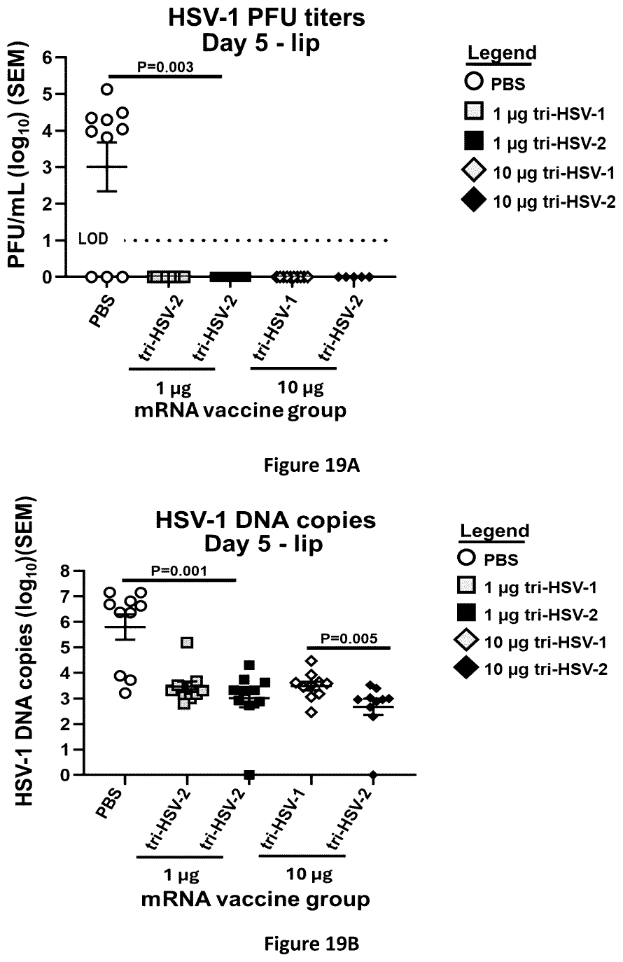

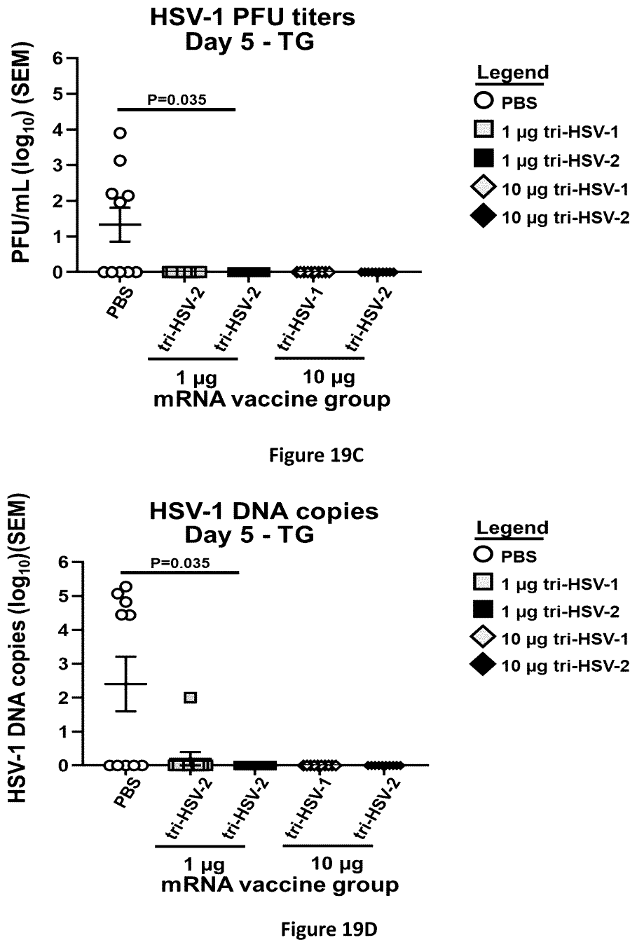

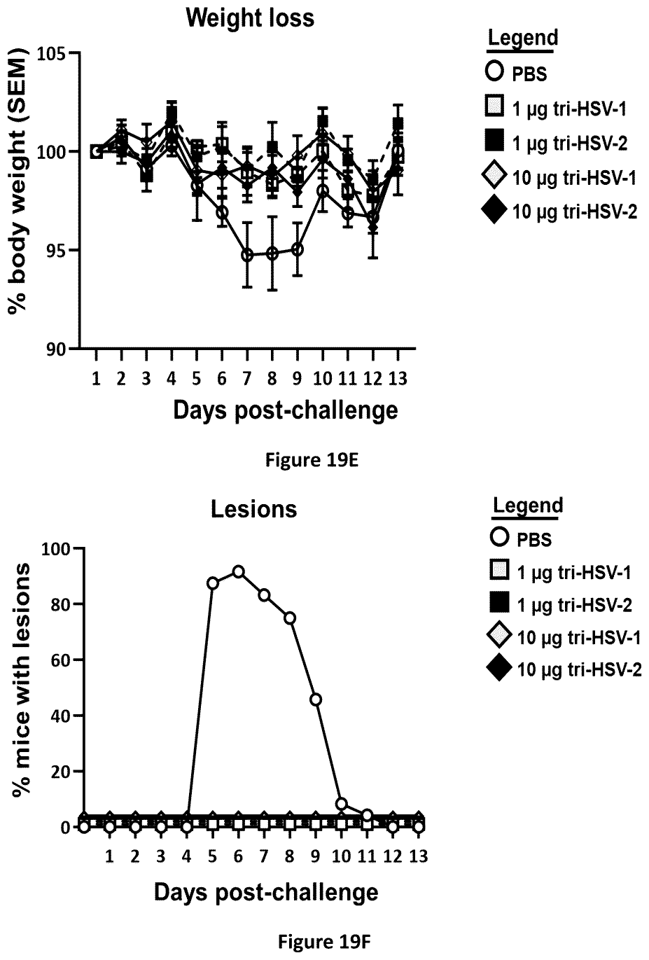

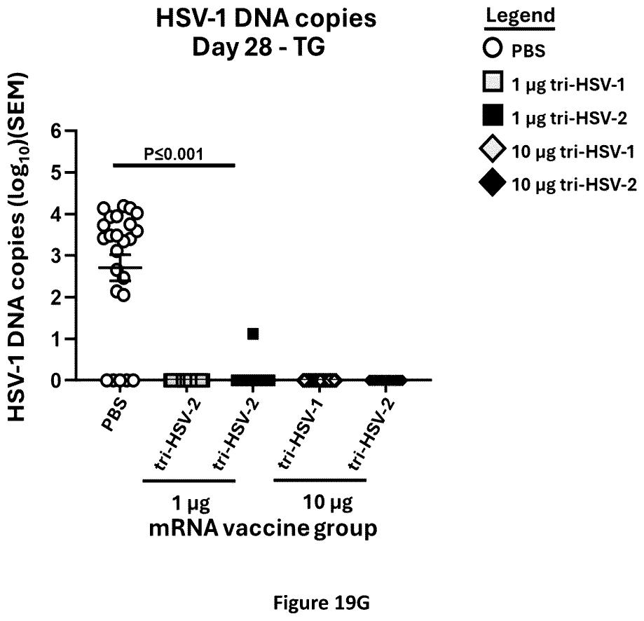

A challenge model that targets anon-genital site for infection was selected to compare efficacy of tri-HSV-1 and tri-HSV-2 modified RNA vaccine immunizations. The most common site of HSV-1 infection is in the oral mucosa. The lip scarification model allows virus to be added to the lower lip adjacent to the oral mucosa. Scratch inoculation of the virus on the lower lip causes productive infection in the lip and trigeminal ganglia (TG) infection via HSV-1 transport along the mandibular branch of the trigeminal nerve. Mice were immunized and later infected by scratch inoculation of 2×10⁶ PFU of HSV-1 strain NS to the lower lip. PBS-immunized mice had high levels of replication-competent virus in the lip indicating productive infection; however, replication-competent virus was not detected in the lip from any immunized mouse (FIG. 19A). By qPCR, the PBS mice had an average 5.8 log₁₀ of HSV-1 DNA present. All vaccinated mice had at least a 1.6-fold reduction in HSV-1 DNA copies. The 10 μg tri-HSV-2 modified RNA vaccine immunized group had a significantly lower copy number of viral DNA compared to the 10 μg tri-HSV-1 modified RNA vaccine immunized group (FIG. 19B). Infectious virus in the TG was measured 5 days post-challenge. Five of 10 (50%) PBS mice had replication competent virus present in the TG, while no immunized mouse had replication competent virus (FIG. 19C). The presence of replication competent virus or HSV-1 DNA in the TG indicates a risk of virus establishing a latent infection. Four of 10 (40%) of the PBS mice had viral DNA in the TG at 5 days (FIG. 19D). Only 1/40 (2.5%) tri-HSV-1 or tri-HSV-2 modified RNA vaccine immunized mice was positive for viral DNA present in the TG at 5 days. The single positive animal was in the 1-μg group of tri-HSV-1 modified RNA vaccine immunized mice (FIG. 19D).

Mice were monitored for weight loss and lip lesions over 13 days. The PBS vaccinated mice lost approximately 5% body weight between days 6 and 9 post-infection. After day 10, the weight in the PBS mice recovered (FIG. 19E). None of the immunized mice lost more than 3% body weight (FIG. 19E). The PBS mice developed lesions on the lower lip 5 days post-infection. At 6 days, 22/24 (92%) of PBS mice had lower lip lesions. The lesions healed over the next 4 days and were no longer present by day 11 (FIG. 19F). None of the immunized mice developed lip lesions (FIG. 19F). The TGs were analyzed for HSV-1 DNA copy number 28 days post-challenge as an indicator of latent HSV-1 infection. HSV-1 DNA in the TG was detected from 79% of the PBS mice (FIG. 19G). Only a single vaccinated mouse in the 1 μg dose tri-HSV-2 modified RNA vaccine immunized group had HSV-1 DNA detected in the TG. The mean copy number of HSV-1 DNA in PBS mice was 2.7 log₁₀ whereas the HSV-1 DNA copy number in the single breakthrough case in the tri-HSV-2 modified RNA vaccine immunized mouse was 1.1 log₁₀, a 2.5-fold reduction. These data suggest that the tri-HSV-1 and tri-HSV-2 modified RNA vaccines provide excellent protection against high-dose HSV-1 challenge in the lip. Both vaccines were nearly perfect in preventing HSV-1 DNA from reaching the TG at acute and latent time points. The tri-HSV-1 and tri-HSV-2 modified RNA vaccines had equivalent efficacy, even at a low dose of 1 μg total RNA.

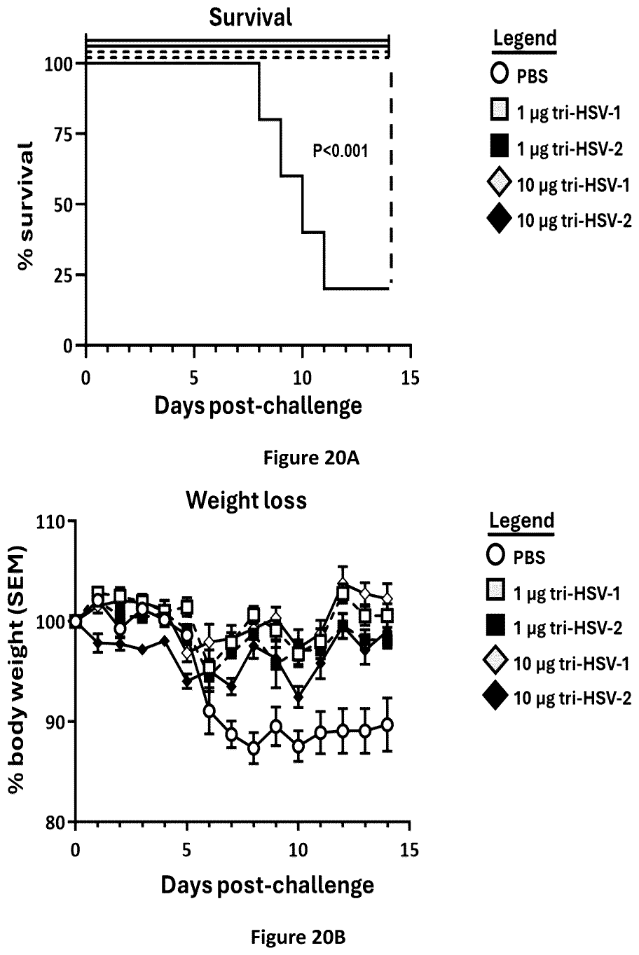

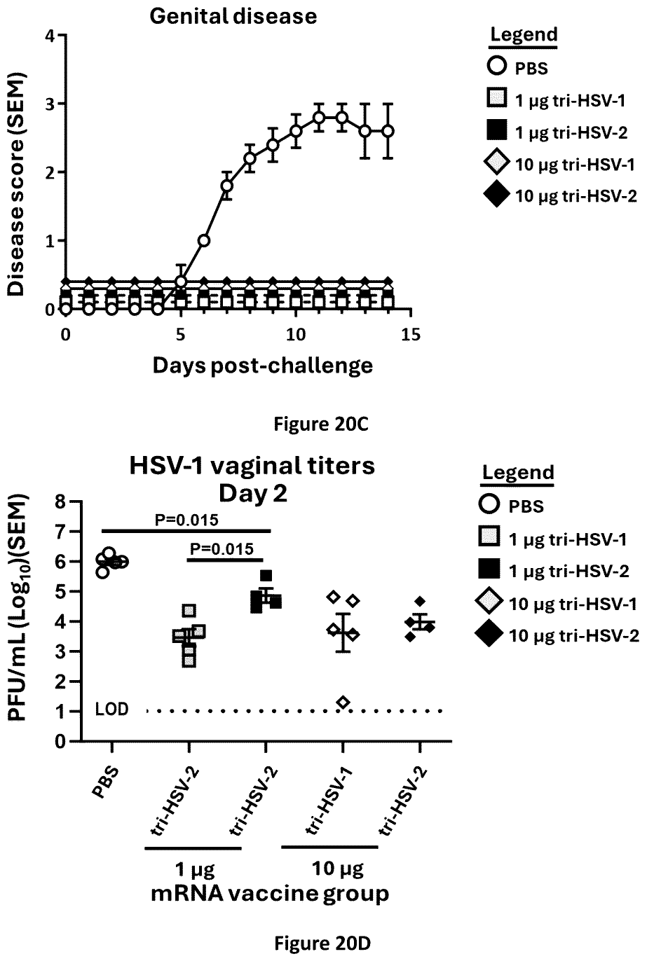

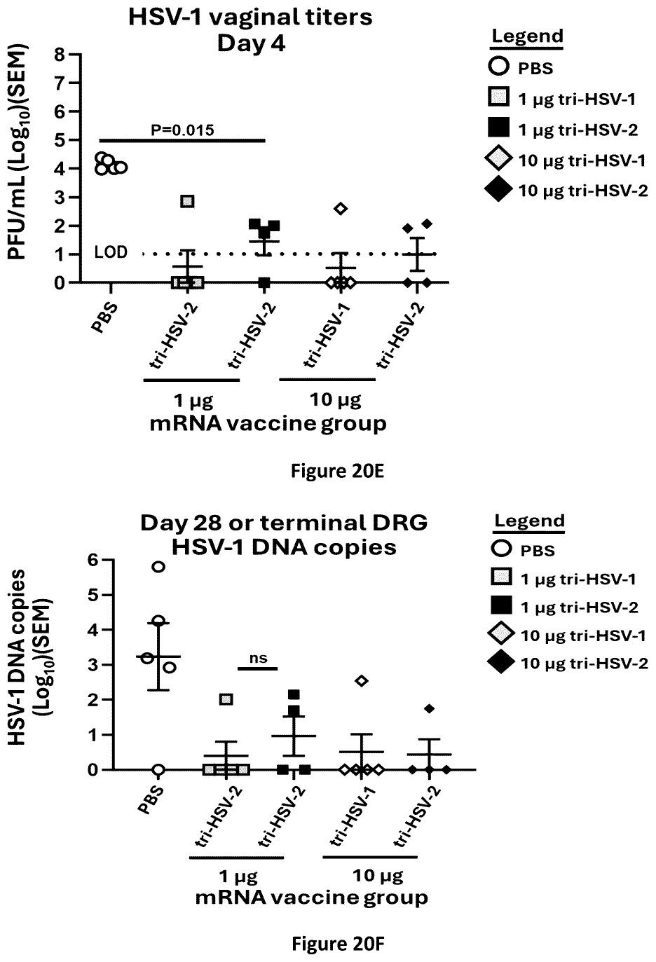

Previously, it had been observed that 16/29 (55%) mice immunized with tri-HSV-2 modified RNA vaccine had positive day 2 vaginal virus titers (mean titer 1.3 PFU/mL) after HSV-1 intravaginal challenge. An evaluation of whether the tri-HSV-1 modified RNA vaccine was better than the tri-HSV-2 modified RNA vaccine in reducing day 2 or day 4 vaginal virus titers after HSV-1 challenge was performed. Mice were vaccinated with tri-HSV-1 and tri-HSV-2 modified RNA vaccines at low (1 μg) and high (10 μg) doses, treated with Depo-Provera five days before infection, and challenged with 2×10⁶ PFU of HSV-1 strain NS. The PBS immunized animals started succumbing to infection by day 8. By day 14, only 1 of 5 (20%) survived the HSV-1 challenge, while none of the tri-HSV-1 or tri-HSV-2 modified RNA vaccinated mice died, at either 1 μg or 10 μg doses (FIG. 20A). In the PBS group, mice started to lose weight by 6 days post-challenge, while the tri-HSV-1 and tri-HSV-2 modified RNA vaccine immunized mice had little or no weight loss (FIG. 20B). Mice were scored for genital disease over 14 days. The PBS vaccinated mice developed genital disease on day 5 post-challenge that peaked on day 11 with a mean disease score of 2.8. Genital disease was not observed in any vaccinated mouse (FIG. 20C).

An evaluation of whether infectious virus was present in vaginal swabs obtained on days 2 or 4 post-challenge was performed. High titers of HSV-1 were detected in all the PBS animals, with a mean titer of 10⁶ PFU/mL. Virus titers were significantly lower in all vaccine groups on days 2 (FIG. 20D) and 4 (FIG. 20E). Importantly, day 2 virus titers for the 1 μg tri-HSV-1 modified RNA vaccine immunized group were approximately 2 log₁₀ lower than the 1 μg tri-HSV-2 modified RNA vaccine immunized group. This difference was the only statistically significant difference detected favoring the tri-HSV-1 modified RNA vaccine in this study and was limited to the 1 μg dose, while at the 10 μg dose in the lip model, the opposite result was observed (FIG. 19B), i.e., there was a lower HSV-1 DNA copy number in the vaginas of tri-HSV-2 modified RNA vaccine immunized mice than tri-HSV-1 modified RAN vaccine immunized mice (FIGS. 19B and 20D). Significant reductions in viral titers were observed in all vaccine groups compared to PBS at 4 days post-challenge with no difference between the tri-HSV-1 and tri-HSV2 modified RNA vaccine immunized groups (FIG. 20E). HSV-1 DNA copy number present in the DRG at the time of humane sacrifice (PBS group) or 28 days post-challenge was evaluated. High levels of HSV-1 DNA were detected in the four PBS animals that were humanely euthanized, while the one surviving animal did not have HSV-1 DNA detected in the DRG at the end of the experiment (FIG. 20F). No significant differences were found between the tri-HSV-1 and tri-HSV-2 modified RNA vaccine immunized groups. These results demonstrate that both the tri-HSV-1 and tri-HSV-2 modified RNA vaccines protected mice against a high dose HSV-1 intravaginal challenge, with only a very small difference favoring the tri-HSV-1 modified RNA vaccine immunized group for day 2 vaginal virus titers at the 1 μg dose.

### Discussion

An effective prophylactic vaccine is the best approach to limit new genital and non-genital HSV infections. The gC2, gD2, and gE2 amino acid sequences share 65%, 82%, and 73% identity with gC1, gD1, and gE1, respectively. An evaluation was performed to determine whether this level of identity was sufficient to generate immune responses capable of potent cross-protection against HSV-1 infection at non-genital sites in mice and whether the protection provided by a tri-HSV-1 vaccine against genital HSV-1 infection in mice can be improved.

The most common site of HSV-1 infection is orolabial. In contrast, HSV-2 rarely infects the oral cavity, suggesting that HSV-1 has a fitness advantage at this site. The lip scarification model of HSV-1 infection was chosen to compare immunization efficacy of the exemplary tri-HSV-2 modified RNA vaccine with an exemplary tri-HSV-1 modified RNA vaccine. In this model, the epithelial surface is scratched to allow the virus to infect the underlying susceptible cells. The tri-HSV-2 RNA vaccine immunization provided total protection against clinical and subclinical infection. Importantly, tri-HSV-2 modified RNA vaccine immunized mice were completely protected against HSV-1 DNA reaching the TG, indicating a minimal risk of latency and reactivation.

An assessment was performed to determine whether the tri-HSV-1 modified RNA vaccine provides better protection than the tri-HSV-2 modified RNA vaccine in the murine genital infection model, particularly against subclinical infection measured by day 2 and day 4 vaginal virus titers. A difference in vaginal titers was observed when comparing the 1 g dose of tri-HSV-1 modified RNA vaccine with 1 g tri-HSV-2 modified RNA vaccine on day 2, but no other differences were noted.

In mice, T-cells play a vital role in restricting the ability of HSV-1 or HSV-2 to reactivate from latency. Mice immunized with tri-HSV-2 modified RNA were shown to have robust CD8+ T-cell responses to gE1 overlapping peptides. Surprisingly, the responses generated by tri-HSV-2 modified RNA vaccine immunized mice were stronger than by tri-HSV-1 modified RNA vaccine immunized mice as manifest by an increase of polyfunctional IFNγ⁺TNFα⁺IL-2⁺CD8⁺ T-cells and an increase in CD107α⁺CD8⁺ T-cells. A greater CD4+ T cell response to gD1 overlapping peptides was detected in tri-HSV-2 modified RNA vaccine immunized mice than tri-HSV-1 modified RNA vaccine immunized mice. These T cell responses may translate into the tri-HSV-2 modified RNA vaccine having an advantage over the tri-HSV-1 modified RNA vaccine in preventing HSV-1 reactivation from latency. This advantage may be more apparent in the guinea pig genital infection model than the mouse model because recurrent genital lesions and recurrent shedding of HSV DNA can be detected in the guinea pig model. Although antibody responses to gC1 and gE1 were lower for the tri-HSV-2 modified RNA vaccine than the tri-HSV-1 modified RNA vaccine, the T-cell responses were more robust. The potent CD8+ T cell responses reveal an unexpected benefit of the tri-HSV-2 modified RNA vaccine against HSV-1 infection.

### Example 19: Evaluation of the HSV-2 Trivalent Nucleoside-Modified RNA/LNP Vaccine Against HSV-1 Encephalitis in Mice

HSV-1 is the most common cause of sporadic infectious encephalitis worldwide and carries approximately a 30% mortality and 60% to 70% morbidity in survivors despite treatment with acyclovir. A vaccine that prevents HSV-1 encephalitis would represent a major advance.

Female BALB/c mice at age 6-8 weeks were immunized intramuscularly in a hind limb twice at one-month intervals with PBS (control), with 1 g trivalent HSV-2 RNA vaccine containing 0.33 g each of exemplary gC2, gD2, gE2 nucleoside-modified RNA in lipid nanoparticle (LNP), or with 10 g trivalent HSV-2 RNA vaccine containing 3.33 g each of exemplary gC2, gD2, gE2 nucleoside-modified RNA in LNP. One month after the second immunization, mice were infected intranasally with 5×10⁵ PFU of HSV-1 (100 LD₅₀) and evaluated for survival, neurologic disease, and for HSV-1 DNA copy number by qPCR in brain, trigeminal ganglia, and olfactory bulb tissues harvested post-infection.

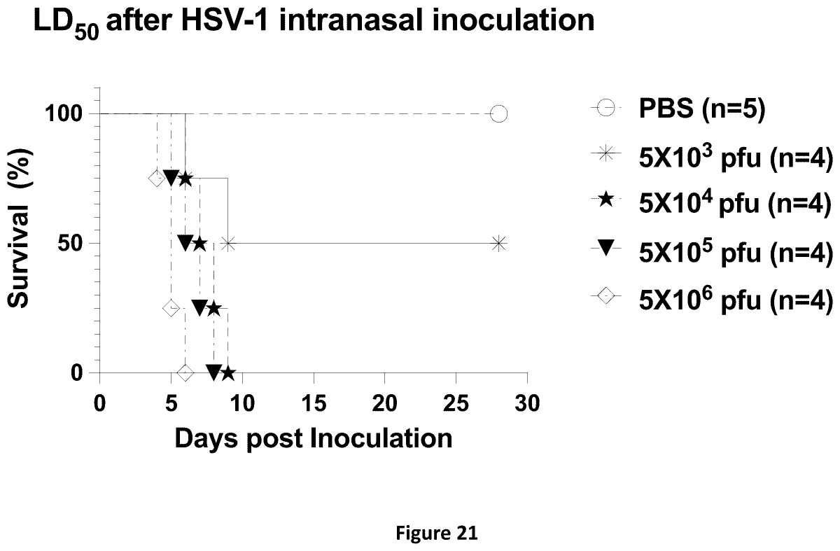

Survival. Naïve (not vaccinated) 6 to 8-week-old female mice were inoculated intranasally with 5×10³, 5×10⁴, 5×10⁵, 5×10⁶ plaque-forming units (pfu) of HSV-1 strain H129, or with PBS as a control. Survival was evaluated over 28 days. All mice in the PBS group survived, while all mice in the groups inoculated with 5×10⁴, 5×10⁵, or 5×10⁶ pfu required humane euthanasia (FIG. 21). Survival was 50% (2 of 4 mice) at 5×10³ pfu, establishing the LD₅₀ as 5×10³ pfu (FIG. 21).

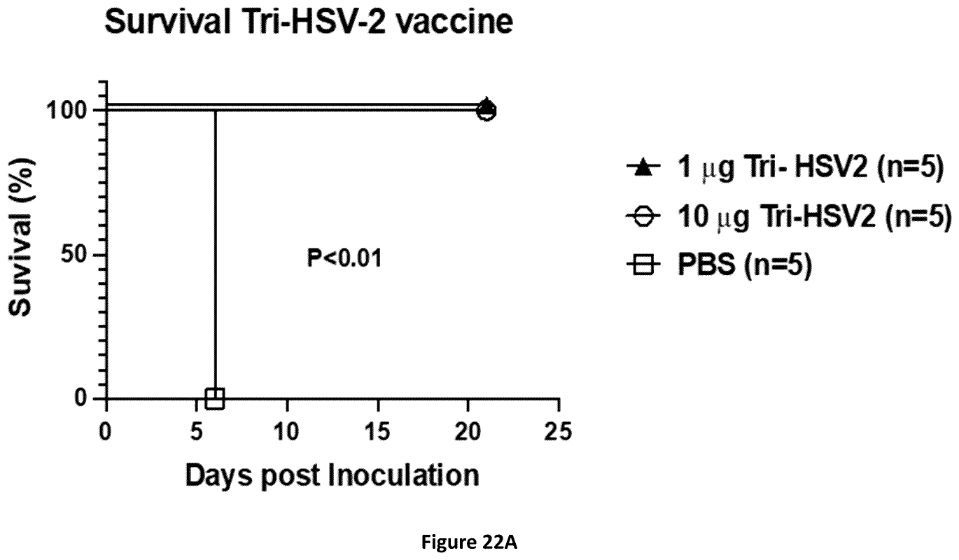

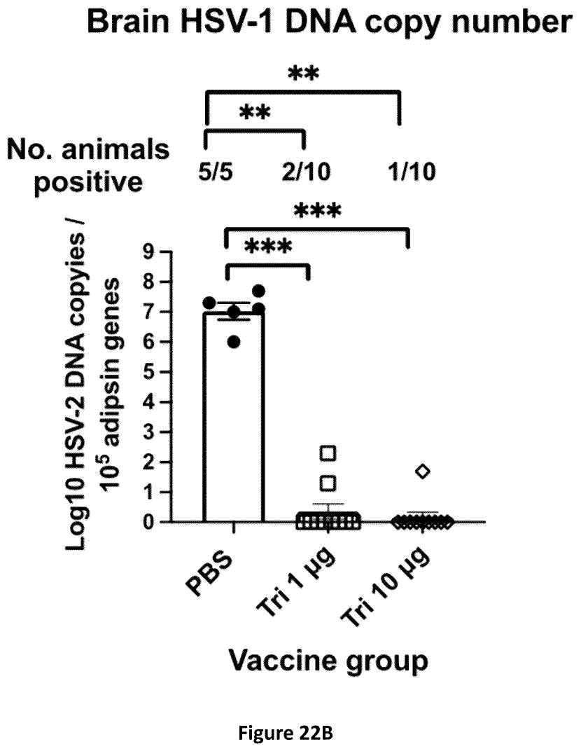

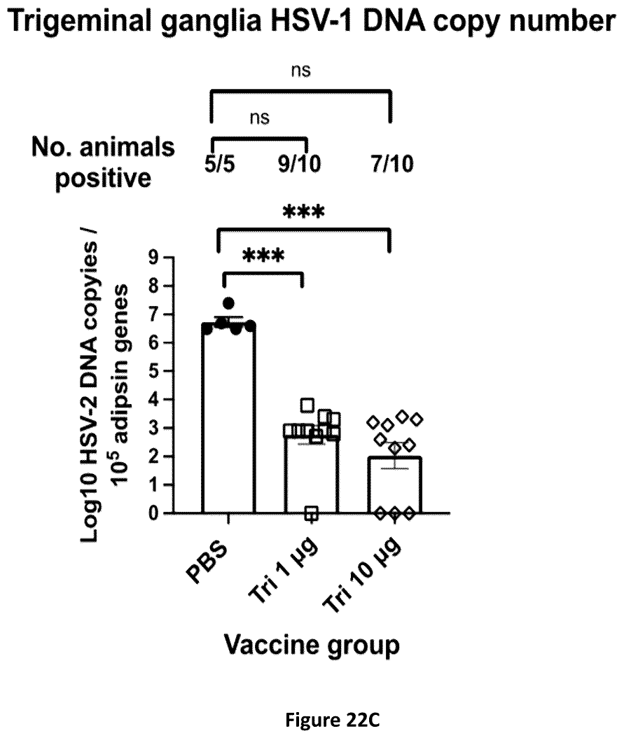

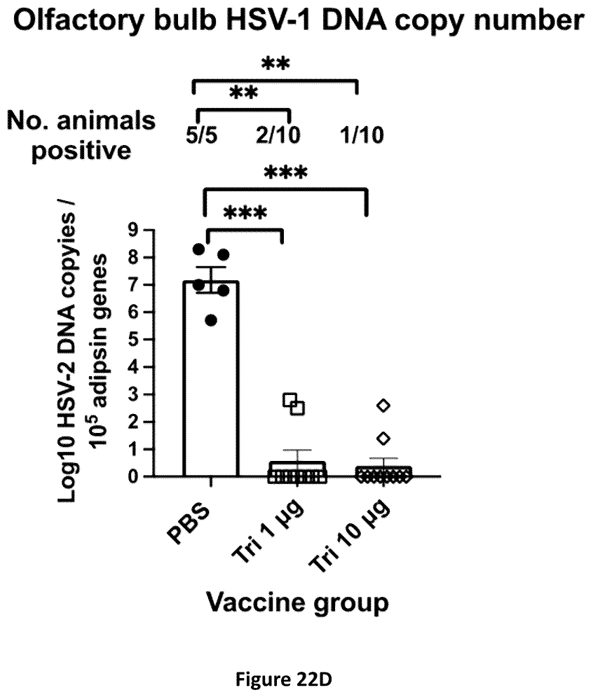

Trivalent HSV-2 vaccine protects BALB/c mice against HSV-1 encephalitis. All 10 animals in the vaccine group immunized with 1 g or 10 g of the tri-HSV-2 RNA-LNP survived, while all 10 mice in the PBS group required humane euthanasia on day 6 post-infection (FIG. 22A). HSV-1 DNA was detected in brains in 5 of 5 (100%) mice in the PBS group with a mean titer of 7 log₁₀ HSV-1 DNA copies per 105 adipsin genes, while HSV-1 DNA was detected in 2 of 10 (20%) mice in the 1 g group with a mean titer of 0.4 log₁₀, or 1 of 10 (10%) mice in the 10 g group with a mean titer of 0.2 log₁₀ (FIG. 22B). HSV-1 DNA was detected in the trigeminal ganglia in 5 of 5 (100%) mice from the PBS group at a mean titer of 6.7 log₁₀, while HSV-1 DNA was detected in 9 of 10 (90%) mice in the 1 g group with a mean titer of 2.8 log₁₀, or 7 of 10 (70%) mice in the 10 g group with a mean titer of 2.0 log₁₀ (FIG. 22C). HSV-1 DNA was detected in the olfactory bulb of mice in the PBS group in 5 of 5 (100%) mice at a mean titer of 7.2 log₁₀, while HSV-1 DNA was detected in 2 of 9 (22%) mice in the 1 g group with a mean titer of 0.6 log₁₀, or 2 of 10 (20%) mice in the 10 g group with a mean titer of 0.4 log₁₀ (FIG. 22D).

Conclusions: The 1 and 10 g doses of the trivalent HSV-2 vaccine were both highly efficacious at protecting mice against death and preventing HSV-1 DNA from reaching the brain and the olfactory bulb. The vaccine was less effective at preventing HSV-1 DNA from reaching the trigeminal ganglia, although the vaccine groups had significantly reduced HSV-1 DNA copy number in the trigeminal ganglia compared with the PBS group. The HSV-2 nucleoside-modified RNA-LNP vaccine is highly efficacious at preventing HSV-1 encephalitis in a mouse model following a high dose lethal challenge.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

All patent documents and references cited herein are incorporated by reference as if fully set forth.

1. A method of treating or inhibiting a Herpes Simplex Virus-1 (HSV-1) oral infection, HSV encephalitis, or a combination thereof in a subject comprising administering to said subject a composition comprising:
(a) RNA encoding an immunogenic fragment of an HSV glycoprotein D (gD);
(b) RNA encoding an immunogenic fragment of an HSV glycoprotein C (gC); and
(c) RNA encoding an immunogenic fragment of an HSV glycoprotein E (gE);
wherein one or more of said RNAs is a nucleoside modified RNA.

2. The method of claim 1, wherein the composition comprises:
(i) RNA encoding an immunogenic fragment of an HSV-1 glycoprotein D (gD); RNA encoding an immunogenic fragment of an HSV-1 glycoprotein C (gC); and RNA encoding an immunogenic fragment of an HSV-1 glycoprotein E (gE); or
(ii) RNA encoding an immunogenic fragment of an HSV-2 glycoprotein D (gD); RNA encoding an immunogenic fragment of an HSV-2 glycoprotein C (gC); and RNA encoding an immunogenic fragment of an HSV-2 glycoprotein E (gE).

3. The method of claim 2, wherein:
(a) the immunogenic fragment of an HSV-1 gC comprises an amino acid sequence that is at least 90% identical to SEQ ID NO: 8 or SEQ ID NO: 9;
(b) the immunogenic fragment of an HSV-1 gD or immunogenic fragment thereof comprises an amino acid sequence at least 90% identical to SEQ ID NO: 2 or SEQ ID NO: 3; and
(c) the immunogenic fragment of an HSV-1 gE or immunogenic fragment thereof comprises an amino acid sequence at least 90% identical to SEQ ID NO: 14 or SEQ ID NO: 15.

4. The method of claim 2, wherein:
(a) the immunogenic fragment of an HSV-2 gD comprises an amino acid sequence that is at least 90% identical to SEQ ID NO: 5;
(b) the immunogenic fragment of an HSV-2 gC or immunogenic fragment thereof comprises an amino acid sequence at least 90% identical to SEQ ID NO: 11; and
(c) the immunogenic fragment of an HSV-2 gE or immunogenic fragment thereof comprises an amino acid sequence at least 90% identical to SEQ ID NO: 17.

5. The method of claim 1, wherein said infection is a primary infection or a secondary infection.

6. The method of claim 1, wherein said nucleoside modified RNA comprises one or more pseudouridine residues.

7. The method of claim 6, wherein said one or more pseudouridine residues comprise m¹Ψ (1-methylpseudouridine), m¹acp³Ψ (1-methyl-3-(3-amino-5-carboxypropyl)pseudouridine, Ψm (2′-O-methylpseudouridine, m5D (5-methyldihydrouridine), m³Ψ (3-methylpseudouridine), or any combination thereof.

8. The method of claim 1, wherein one or more of said RNAs further comprises a signal sequence encoding a signal peptide.

9. The method of claim 8, wherein the composition comprises:
(a) RNA encoding an immunogenic fragment of an HSV gD and a signal sequence;
(b) RNA encoding an immunogenic fragment of an HSV gC and a signal sequence; and
(c) RNA encoding an immunogenic fragment of an HSV gE and a signal sequence.

10. The method of claim 1, wherein one or more of said RNAs further comprise:
(i) a poly-A tail;
(ii) an m7GpppG cap, 3′-O-methyl-m7GpppG cap, or anti-reverse cap analog;
(iii) a cap-independent translational enhancer; and/or
(iv) 5′ and/or 3′ untranslated regions that enhance translation.

11. The method of claim 1, wherein one or more of said RNAs are formulated with a nanoparticle, lipid, polymer, cholesterol, cell penetrating peptide, or any combination thereof.

12. The method of claim 11, wherein said nanoparticle is a lipid nanoparticle (LNP).

13. The method of claim 1, wherein the administration step comprises intramuscular, subcutaneous, intradermal, intranasal, intravaginal, intrarectal, or topical administration.

14. The method of claim 1 comprising a method of treating or inhibiting an HSV-1 oral infection in a subject comprising administering to said subject a composition comprising:
(a) RNA encoding an immunogenic fragment of an HSV glycoprotein D (gD);
(b) RNA encoding an immunogenic fragment of an HSV glycoprotein C (gC); and
(c) RNA encoding an immunogenic fragment of an HSV glycoprotein E (gE);
wherein one or more of said RNAs is a nucleoside modified RNA.

15. The method of claim 14, wherein:
(a) the immunogenic fragment of an HSV gD comprises an amino acid sequence that is at least 90% identical to SEQ ID NO: 2 or SEQ ID NO: 5;
(b) the immunogenic fragment of an HSV gD or immunogenic fragment thereof comprises an amino acid sequence at least 90% identical to SEQ ID NO: 8 or SEQ ID NO: 11; and
(c) the immunogenic fragment of an HSV gE or immunogenic fragment thereof comprises an amino acid sequence at least 90% identical to SEQ ID NO: 14 or SEQ ID NO: 17.

16. The method of claim 1 comprising a method of treating or inhibiting HSV encephalitis in a subject comprising administering to said subject a composition comprising:
(a) RNA encoding an immunogenic fragment of an HSV glycoprotein D (gD);
(b) RNA encoding an immunogenic fragment of an HSV glycoprotein C (gC); and
(c) RNA encoding an immunogenic fragment of an HSV glycoprotein E (gE);
wherein one or more of said RNAs is a nucleoside modified RNA.

17. The method of claim 16, wherein the HSV encephalitis is caused by HSV-1.

18. The method of claim 16, wherein:
(a) the immunogenic fragment of an HSV gD comprises an amino acid sequence that is at least 90% identical to SEQ ID NO: 5;
(b) the immunogenic fragment of an HSV gD or immunogenic fragment thereof comprises an amino acid sequence at least 90% identical to SEQ ID NO: 11; and
(c) the immunogenic fragment of an HSV gE or immunogenic fragment thereof comprises an amino acid sequence at least 90% identical to SEQ ID NO: 17.


SEQUENCE LISTING (27 sequences)

SEQ ID NO: 1 - RNA, 1386 nt, synthetic construct
  source 1..1386: mol_type = other RNA
ggaataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcatgcgcatgcagctgctgctgctgatcgccctgtccctggccctggtgaccaactccaagtacgccctggccgacgcctccctgaagatggccgaccccaaccgcttccgcggcaaggacctgcccgtgctggaccagctgaccgacccccccggcgtgcgccgcgtgtaccacatccaggccggcctgcccgaccccttccagcccccctccctgcccatcaccgtgtactacgccgtgctggagcgcgcctgccgctccgtgctgctgaacgccccctccgaggccccccagatcgtgcgcggcgcctccgaggacgtgcgcaagcagccctacaacctgaccatcgcctggttccgcatgggcggcaactgcgccatccccatcaccgtgatggagtacaccgagtgctcctacaacaagtccctgggcgcctgccccatccgcacccagccccgctggaactactacgactccttctccgccgtgtccgaggacaacctgggcttcctgatgcacgcccccgccttcgagaccgccggcacctacctgcgcctggtgaagatcaacgactggaccgagatcacccagttcatcctggagcaccgcgccaagggctcctgcaagtacgccctgcccctgcgcatccccccctccgcctgcctgtccccccaggcctaccagcagggcgtgaccgtggactccatcggcatgctgccccgcttcatccccgagaaccagcgcaccgtggccgtgtactccctgaagatcgccggctggcacggccccaaggccccctacacctccaccctgctgccccccgagctgtccgagacccccaacgccacccagcccgagctggcccccgaggaccccgaggactccgccctgctggaggaccccgtgggcaccgtggccccccagatcccccccaactggcacatcccctccatccaggacgccgccaccccctactaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaac

SEQ ID NO: 2 - protein, 306 aa, Human alphaherpesvirus 1
KYALADASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAPQIPPNWHIPSIQDAATPY

SEQ ID NO: 3 - protein, 394 aa, Human alphaherpesvirus 1
MGGAAARLGAVILFVVIVGLHGVRGKYALADASLKLADPNRFRRKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEAPEDSALLEDPVGTVAPQIPPNWHIPSIQDAATPYHPPATPNNMGLIAGAVGGSLLAALVICGIVYWMRRRTQKAPKRIRLPHIREDDQPSSHQPLFY

SEQ ID NO: 4 - RNA, 1386 nt, synthetic construct
  source 1..1386: mol_type = other RNA
ggaataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcatgacccgcctgaccgtgctggccctgctggccggcctgctggcctcctcccgcgccaagtacgccctggccgacccctccctgaagatggccgaccccaaccgcttccgcggcaagaacctgcccgtgctggaccagctgaccgacccccccggcgtgaagcgcgtgtaccacatccagccctccctggaggaccccttccagcccccctccatccccatcaccgtgtactacgccgtgctggagcgcgcctgccgctccgtgctgctgcacgccccctccgaggccccccagatcgtgcgcggcgcctccgacgaggcccgcaagcacacctacaacctgaccatcgcctggtaccgcatgggcgacaactgcgccatccccatcaccgtgatggagtacaccgagtgcccctacaacaagtccctgggcgtgtgccccatccgcacccagccccgctggtcctactacgactccttctccgccgtgtccgaggacaacctgggcttcctgatgcacgcccccgccttcgagaccgccggcacctacctgcgcctggtgaagatcaacgactggaccgagatcacccagttcatcctggagcaccgcgcccgcgcctcctgcaagtacgccctgcccctgcgcatcccccccgccgcctgcctgacctccaaggcctaccagcagggcgtgaccgtggactccatcggcatgctgccccgcttcatccccgagaaccagcgcaccgtggccctgtactccctgaagatcgccggctggcacggccccaagcccccctacacctccaccctgctgccccccgagctgtccgacaccaccaacgccacccagcccgagctggtgcccgaggaccccgaggactccgccctgctggaggaccccgccggcaccgtgtcctcccagatcccccccaactggcacatcccctccatccaggacgtggccccccaccactaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaac

SEQ ID NO: 5 - protein, 306 aa, Human alphaherpesvirus 2
KYALADPSLKMADPNRFRGKNLPVLDQLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHH

SEQ ID NO: 6 - protein, 393 aa, Human alphaherpesvirus 2
MGRLTSGVGTAALLVVAVGLRVVCAKYALADPSLKMADPNRFRGKNLPVLDQLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHHAPAAPSNPGLIIGALAGSTLAVLVIGGIAFWVRRRAQMAPKRLRLPHIRDDDAPPSHQPLFY

SEQ ID NO: 7 - RNA, 1779 nt, synthetic construct
  source 1..1779: mol_type = other RNA
ggaataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcatggccatctccggcgtgcccgtgctgggcttcttcatcatcgccgtgctgatgtccgcccaggagtcctgggccgagaccgcctccaccggccccaccatcaccgccggcgccgtgaccaacgcctccgaggcccccacctccggctcccccggctccgccgcctcccccgaggtgacccccacctccacccccaaccccaacaacgtgacccagaacaagaccacccccaccgagcccgcctccccccccaccacccccaagcccacctccacccccaagtccccccccacctccacccccgaccccaagcccaagaacaacaccacccccgccaagtccggccgccccaccaagccccccggccccgtgtggtgcgaccgccgcgaccccctggcccgctacggctcccgcgtgcagatccgctgccgcttccgcaactccacccgcatggagttccgcctgcagatctggcgctactccatgggcccctccccccccatcgcccccgcccccgacctggaggaggtgctgaccaacatcaccgccccccccggcggcctgctggtgtacgactccgcccccaacctgaccgacccccacgtgctgtgggccgagggcgccggccccggcgccgacccccccctgtactccgtgaccggccccctgcccacccagcgcctgatcatcggcgaggtgacccccgccacccagggcatgtactacctggcctggggccgcatggactccccccacgagtacggcacctgggtgcgcgtgcgcatgttccgccccccctccctgaccctgcagccccacgccgtgatggagggccagcccttcaaggccacctgcaccgccgccgcctactacccccgcaaccccgtggagttcgactggttcgaggacgaccgccaggtgttcaaccccggccagatcgacacccagacccacgagcaccccgacggcttcaccaccgtgtccaccgtgacctccgaggccgtgggcggccaggtgcccccccgcaccttcacctgccagatgacctggcaccgcgactccgtgaccttctcccgccgcaacgccaccggcctggccctggtgctgccccgccccaccatcaccatggagttcggcgtgcgccacgtggtgtgcaccgccggctgcgtgcccgagggcgtgaccttcgcctggttcctgggcgacgacccctcccccgccgccaagtccgccgtgaccgcccaggagtcctgcgaccaccccggcctggccaccgtgcgctccaccctgcccatctcctacgactactccgagtacatctgccgcctgaccggctaccccgccggcatccccgtgctggagcaccactaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaac

SEQ ID NO: 8 - protein, 431 aa, Human alphaherpesvirus 1
ETASTGPTITAGAVTNASEAPTSGSPGSAASPEVTPTSTPNPNNVTQNKTTPTEPASPPTTPKPTSTPKSPPTSTPDPKPKNNTTPAKSGRPTKPPGPVWCDRRDPLARYGSRVQIRCRFRNSTRMEFRLQIWRYSMGPSPPIAPAPDLEEVLTNITAPPGGLLVYDSAPNLTDPHVLWAEGAGPGADPPLYSVTGPLPTQRLIIGEVTPATQGMYYLAWGRMDSPHEYGTWVRVRMFRPPSLTLQPHAVMEGQPFKATCTAAAYYPRNPVEFDWFEDDRQVFNPGQIDTQTHEHPDGFTTVSTVTSEAVGGQVPPRTFTCQMTWHRDSVTFSRRNATGLALVLPRPTITMEFGVRHVVCTAGCVPEGVTFAWFLGDDPSPAAKSAVTAQESCDHPGLATVRSTLPISYDYSEYICRLTGYPAGIPVLEHH

SEQ ID NO: 9 - protein, 511 aa, Human alphaherpesvirus 1
MAPGRVGLAVVLWGLLWLGAGVAGGSETASTGPTITAGAVTNASEAPTSGSPGSAASPEVTPTSTPNPNNVTQNKTTPTEPASPPTTPKPTSTPKSPPTSTPDPKPKNNTTPAKSGRPTKPPGPVWCDRRDPLARYGSRVQIRCRFRNSTRMEFRLQIWRYSMGPSPPIAPAPDLEEVLTNITAPPGGLLVYDSAPNLTDPHVLWAEGAGPGADPPLYSVTGPLPTQRLIIGEVTPATQGMYYLAWGRMDSPHEYGTWVRVRMFRPPSLTLQPHAVMEGQPFKATCTAAAYYPRNPVEFDWFEDDRQVFNPGQIDTQTHEHPDGFTTVSTVTSEAVGGQVPPRTFTCQMTWHRDSVTFSRRNATGLALVLPRPTITMEFGVRHVVCTAGCVPEGVTFAWFLGDDPSPAAKSAVTAQESCDHPGLATVRSTLPISYDYSEYICRLTGYPAGIPVLEHHGSHQPPPRDPTERQVIEAIEWVGIGIGVLAAGVLVVTAIVYVVRTSQSRQRHRR

SEQ ID NO: 10 - RNA, 1668 nt, synthetic construct
  source 1..1668: mol_type = other RNA
ggaataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcatgcgcatgcagctgctgctgctgatcgccctgtccctggccctggtgaccaactccgcctcccccggccgcaccatcaccgtgggcccccgcggcaacgcctccaacgccgccccctccgcctccccccgcaacgcctccgccccccgcaccacccccacccccccccagccccgcaaggccaccaagtccaaggcctccaccgccaagcccgcccccccccccaagaccggcccccccaagacctcctccgagcccgtgcgctgcaaccgccacgaccccctggcccgctacggctcccgcgtgcagatccgctgccgcttccccaactccacccgcaccgagttccgcctgcagatctggcgctacgccaccgccaccgacgccgagatcggcaccgccccctccctggaggaggtgatggtgaacgtgtccgccccccccggcggccagctggtgtacgactccgcccccaaccgcaccgacccccacgtgatctgggccgagggcgccggccccggcgcctccccccgcctgtactccgtggtgggccccctgggccgccagcgcctgatcatcgaggagctgaccctggagacccagggcatgtactactgggtgtggggccgcaccgaccgcccctccgcctacggcacctgggtgcgcgtgcgcgtgttccgccccccctccctgaccatccacccccacgccgtgctggagggccagcccttcaaggccacctgcaccgccgccacctactaccccggcaaccgcgccgagttcgtgtggttcgaggacggccgccgcgtgttcgaccccgcccagatccacacccagacccaggagaaccccgacggcttctccaccgtgtccaccgtgacctccgccgccgtgggcggccagggccccccccgcaccttcacctgccagctgacctggcaccgcgactccgtgtccttctcccgccgcaacgcctccggcaccgcctccgtgctgccccgccccaccatcaccatggagttcaccggcgaccacgccgtgtgcaccgccggctgcgtgcccgagggcgtgaccttcgcctggttcctgggcgacgactcctcccccgccgagaaggtggccgtggcctcccagacctcctgcggccgccccggcaccgccaccatccgctccaccctgcccgtgtcctacgagcagaccgagtacatctgccgcctggccggctaccccgacggcatccccgtgctggagcaccactaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaac

SEQ ID NO: 11 - protein, 400 aa, Human alphaherpesvirus 2
ASPGRTITVGPRGNASNAAPSASPRNASAPRTTPTPPQPRKATKSKASTAKPAPPPKTGPPKTSSEPVRCNRHDPLARYGSRVQIRCRFPNSTRTESRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHH

SEQ ID NO: 12 - protein, 480 aa, Human alphaherpesvirus 2
MALGRVGLAVGLWGLLWVGVVVVLANASPGRTITVGPRGNASNAAPSASPRNASAPRTTPTPPQPRKATKSKASTAKPAPPPKTGPPKTSSEPVRCNRHDPLARYGSRVQIRCRFPNSTRTEFRLQIWRYATATDAEIGTAPSLEEVMVNVSAPPGGQLVYDSAPNRTDPHVIWAEGAGPGASPRLYSVVGPLGRQRLIIEELTLETQGMYYWVWGRTDRPSAYGTWVRVRVFRPPSLTIHPHAVLEGQPFKATCTAATYYPGNRAEFVWFEDGRRVFDPAQIHTQTQENPDGFSTVSTVTSAAVGGQGPPRTFTCQLTWHRDSVSFSRRNASGTASVLPRPTITMEFTGDHAVCTAGCVPEGVTFAWFLGDDSSPAEKVAVASQTSCGRPGTATIRSTLPVSYEQTEYICRLAGYPDGIPVLEHHGSHQPPPRDPTERQVIRAVEGAGIGVAVLVAVVLAGTAVVYLTHASSVRYRRLR

SEQ ID NO: 13 - RNA, 1626 nt, synthetic construct
  source 1..1626: mol_type = other RNA
ggaataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcatgcgcatgcagctgctgctgctgatcgccctgtccctggccctggtgaccaactccaagacctcctggcgccgcgtgtccgtgggcgaggacgtgtccctgctgcccgcccccggccccaccggccgcggccccacccagaagctgctgtgggccgtggagcccctggacggctgcggccccctgcacccctcctgggtgtccctgatgccccccaagcaggtgcccgagaccgtggtggacgccgcctgcatgcgcgcccccgtgcccctggccatggcctacgccccccccgccccctccgccaccggcggcctgcgcaccgacttcgtgtggcaggagcgcgccgccgtggtgaaccgctccctggtgatctacggcgtgcgcgagaccgactccggcctgtacaccctgtccgtgggcgacatcaaggaccccgcccgccaggtggcctccgtggtgctggtggtgcagcccgcccccgtgcccacccccccccccacccccgccgactacgacgaggacgacaacgacgagggcgagggcgaggacgagtccctggccggcacccccgcctccggcaccccccgcctgcccccctcccccgcccccccccgctcctggccctccgcccccgaggtgtcccacgtgcgcggcgtgaccgtgcgcatggagacccccgaggccatcctgttctcccccggcgaggccttctccaccaacgtgtccatccacgccatcgcccacgacgaccagacctacaccatggacgtggtgtggctgcgcttcgacgtgcccacctcctgcgccgagatgcgcatctacgagtcctgcctgtaccacccccagctgcccgagtgcctgtcccccgccgacgccccctgcgccgcctccacctggacctcccgcctggccgtgcgctcctacgccggctgctcccgcaccaacccccccccccgctgctccgccgaggcccacatggagcccttccccggcctggcctggcaggccgcctccgtgaacctggagttccgcgacgcctccccccagcactccggcctgtacctgtgcgtggtgtacgtgaacgaccacatccacgcctggggccacatcaccatcaacaccgccgcccagtaccgcaacgccgtggtggagcagcccctgccccagcgcggcgccgacctggccgagcccacccacccccacgtgggcgcctaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaac

SEQ ID NO: 14 - protein, 386 aa, Human alphaherpesvirus 1
KTSWRRVSVGEDVSLLPAPGPTGRGPTQKLLWAVEPLDGCGPLHPSWVSLMPPKQVPETVVDAACMRAPVPLAMAYAPPAPSATGGLRTDFVWQERAAVVNRSLVIYGVRETDSGLYTLSVGDIKDPARQVASVVLVVQPAPVPTPPPTPADYDEDDNDEGEGEDESLAGTPASGTPRLPPSPAPPRSWPSAPEVSHVRGVTVRMETPEAILFSPGEAFSTNVSIHAIAHDDQTYTMDVVWLRFDVPTSCAEMRIYESCLYHPQLPECLSPADAPCAASTWTSRLAVRSYAGCSRTNPPPRCSAEAHMEPFPGLAWQAASVNLEFRDASPQHSGLYLCVVYVNDHIHAWGHITINTAAQYRNAVVEQPLPQRGADLAEPTHPHVGA

SEQ ID NO: 15 - protein, 552 aa, Human alphaherpesvirus 1
MDRGAVVGFLLGVCVVSCLAGTPKTSWRRVSVGEDVSLLPAPGPTGRGPTQKLLWAVEPLDGCGPLHPSWVSLMPPKQVPETVVDAACMRAPVPLAMAYAPPAPSATGGLRTDFVWQERAAVVNRSLVIYGVRETDSGLYTLSVGDIKDPARQVASVVLVVQPAPVPTPPPTPADYDEDDNDEGEGEDESLAGTPASGTPRLPPSPAPPRSWPSAPEVSHVRGVTVRMETPEAILFSPGEAFSTNVSIHAIAHDDQTYTMDVVWLRFDVPTSCAEMRIYESCLYHPQLPECLSPADAPCAASTWTSRLAVRSYAGCSRTNPPPRCSAEAHMEPFPGLAWQAASVNLEFRDASPQHSGLYLCVVYVNDHIHAWGHITINTAAQYRNAVVEQPLPQRGADLAEPTHPHVGAPPHAPPTHGALRLGAVMGAALLLSALGLSVWACMTCWRRRAWRAVKSRASGKGPTYIRVADSELYADWSSDSEGERDQVPWLAPPERPDSPSTNGSGFEILSPTAPSVYPRSDGHQSRRQLTTFGSGRPDRRYSQASDSSVFW

SEQ ID NO: 16 - RNA, 1614 nt, synthetic construct
  source 1..1614: mol_type = other RNA
ggaataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcatgcgcatgcagctgctgctgctgatcgccctgtccctggccctggtgaccaactcccgcacctcctggaagcgcgtgacctccggcgaggacgtggtgctgctgcccgcccccgccggccccgaggagcgcacccgcgcccacaagctgctgtgggccgccgagcccctggacgcctgcggccccctgcgcccctcctgggtggccctgtggcccccccgccgcgtgctggagaccgtggtggacgccgcctgcatgcgcgcccccgagcccctggccatcgcctactccccccccttccccgccggcgacgagggcctgtactccgagctggcctggcgcgaccgcgtggccgtggtgaacgagtccctggtgatctacggcgccctggagaccgactccggcctgtacaccctgtccgtggtgggcctgtccgacgaggcccgccaggtggcctccgtggtgctggtggtggagcccgcccccgtgcccacccccacccccgacgactacgacgaggaggacgacgccggcgtgtccgagcgcacccccgtgtccgtgcccccccccacccccccccgccgcccccccgtggccccccccacccacccccgcgtgatccccgaggtgtcccacgtgcgcggcgtgaccgtgcacatggagacccccgaggccatcctgttcgcccccggcgagaccttcggcaccaacgtgtccatccacgccatcgcccacgacgacggcccctacgccatggacgtggtgtggatgcgcttcgacgtgccctcctcctgcgccgagatgcgcatctacgaggcctgcctgtaccacccccagctgcccgagtgcctgtcccccgccgacgccccctgcgccgtgtcctcctgggcctaccgcctggccgtgcgctcctacgccggctgctcccgcaccaccccccccccccgctgcttcgccgaggcccgcatggagcccgtgcccggcctggcctggctggcctccaccgtgaacctggagttccagcacgcctccccccagcacgccggcctgtacctgtgcgtggtgtacgtggacgaccacatccacgcctggggccacatgaccatctccaccgccgcccagtaccgcaacgccgtggtggagcagcacctgccccagcgccagcccgagcccgtggagcccacccgcccccacgtgcgcgcctaactagtagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaac

SEQ ID NO: 17 - protein, 382 aa, Human alphaherpesvirus 2
RTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRA

SEQ ID NO: 18 - protein, 545 aa, Human alphaherpesvirus 2
MARGAGLVFFVGVWVVSCLAAAPRTSWKRVTSGEDVVLLPAPAERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVTNARRSAFPPQPPPRRPPVAPPTHPRVIPEVSHVRGVTVHMETLEAILFAPGETFGTNVSIHAIAHDDGPYAMDVVWMRFDVPSSCADMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPHPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYPSVLW

SEQ ID NO: 19 - RNA, 57 nt, Human alphaherpesvirus 2
  source 1..57: mol_type = mRNA
atgacccgcctgaccgtgctggccctgctggccggcctgctggcctcctcccgcgcc

SEQ ID NO: 20 - RNA, 57 nt, Homo sapiens
  misc_feature 1..57: note = codon-optimized form of human IL-2
  source 1..57: mol_type = mRNA
atgcgcatgcagctgctgctgctgatcgccctgtccctggccctggtgaccaactcc

SEQ ID NO: 21 - RNA, 75 nt, Homo sapiens
  source 1..75: mol_type = mRNA
atggccatctccggcgtgcccgtgctgggcttcttcatcatcgccgtgctgatgtccgcccaggagtcctgggcc

SEQ ID NO: 22 - RNA, 921 nt, Human alphaherpesvirus 2
  source 1..921: mol_type = mRNA
aagtacgccctggccgacccctccctgaagatggccgaccccaaccgcttccgcggcaagaacctgcccgtgctggaccagctgaccgacccccccggcgtgaagcgcgtgtaccacatccagccctccctggaggaccccttccagcccccctccatccccatcaccgtgtactacgccgtgctggagcgcgcctgccgctccgtgctgctgcacgccccctccgaggccccccagatcgtgcgcggcgcctccgacgaggcccgcaagcacacctacaacctgaccatcgcctggtaccgcatgggcgacaactgcgccatccccatcaccgtgatggagtacaccgagtgcccctacaacaagtccctgggcgtgtgccccatccgcacccagccccgctggtcctactacgactccttctccgccgtgtccgaggacaacctgggcttcctgatgcacgcccccgccttcgagaccgccggcacctacctgcgcctggtgaagatcaacgactggaccgagatcacccagttcatcctggagcaccgcgcccgcgcctcctgcaagtacgccctgcccctgcgcatcccccccgccgcctgcctgacctccaaggcctaccagcagggcgtgaccgtggactccatcggcatgctgccccgcttcatccccgagaaccagcgcaccgtggccctgtactccctgaagatcgccggctggcacggccccaagcccccctacacctccaccctgctgccccccgagctgtccgacaccaccaacgccacccagcccgagctggtgcccgaggaccccgaggactccgccctgctggaggaccccgccggcaccgtgtcctcccagatcccccccaactggcacatcccctccatccaggacgtggccccccaccactaa

SEQ ID NO: 23 - RNA, 1203 nt, Human alphaherpesvirus 2
  source 1..1203: mol_type = mRNA
gcctcccccggccgcaccatcaccgtgggcccccgcggcaacgcctccaacgccgccccctccgcctccccccgcaacgcctccgccccccgcaccacccccacccccccccagccccgcaaggccaccaagtccaaggcctccaccgccaagcccgcccccccccccaagaccggcccccccaagacctcctccgagcccgtgcgctgcaaccgccacgaccccctggcccgctacggctcccgcgtgcagatccgctgccgcttccccaactccacccgcaccgagttccgcctgcagatctggcgctacgccaccgccaccgacgccgagatcggcaccgccccctccctggaggaggtgatggtgaacgtgtccgccccccccggcggccagctggtgtacgactccgcccccaaccgcaccgacccccacgtgatctgggccgagggcgccggccccggcgcctccccccgcctgtactccgtggtgggccccctgggccgccagcgcctgatcatcgaggagctgaccctggagacccagggcatgtactactgggtgtggggccgcaccgaccgcccctccgcctacggcacctgggtgcgcgtgcgcgtgttccgccccccctccctgaccatccacccccacgccgtgctggagggccagcccttcaaggccacctgcaccgccgccacctactaccccggcaaccgcgccgagttcgtgtggttcgaggacggccgccgcgtgttcgaccccgcccagatccacacccagacccaggagaaccccgacggcttctccaccgtgtccaccgtgacctccgccgccgtgggcggccagggccccccccgcaccttcacctgccagctgacctggcaccgcgactccgtgtccttctcccgccgcaacgcctccggcaccgcctccgtgctgccccgccccaccatcaccatggagttcaccggcgaccacgccgtgtgcaccgccggctgcgtgcccgagggcgtgaccttcgcctggttcctgggcgacgactcctcccccgccgagaaggtggccgtggcctcccagacctcctgcggccgccccggcaccgccaccatccgctccaccctgcccgtgtcctacgagcagaccgagtacatctgccgcctggccggctaccccgacggcatccccgtgctggagcaccactaa

SEQ ID NO: 24 - RNA, 1149 nt, Human alphaherpesvirus 2
  source 1..1149: mol_type = mRNA
cgcacctcctggaagcgcgtgacctccggcgaggacgtggtgctgctgcccgcccccgccggccccgaggagcgcacccgcgcccacaagctgctgtgggccgccgagcccctggacgcctgcggccccctgcgcccctcctgggtggccctgtggcccccccgccgcgtgctggagaccgtggtggacgccgcctgcatgcgcgcccccgagcccctggccatcgcctactccccccccttccccgccggcgacgagggcctgtactccgagctggcctggcgcgaccgcgtggccgtggtgaacgagtccctggtgatctacggcgccctggagaccgactccggcctgtacaccctgtccgtggtgggcctgtccgacgaggcccgccaggtggcctccgtggtgctggtggtggagcccgcccccgtgcccacccccacccccgacgactacgacgaggaggacgacgccggcgtgtccgagcgcacccccgtgtccgtgcccccccccacccccccccgccgcccccccgtggccccccccacccacccccgcgtgatccccgaggtgtcccacgtgcgcggcgtgaccgtgcacatggagacccccgaggccatcctgttcgcccccggcgagaccttcggcaccaacgtgtccatccacgccatcgcccacgacgacggcccctacgccatggacgtggtgtggatgcgcttcgacgtgccctcctcctgcgccgagatgcgcatctacgaggcctgcctgtaccacccccagctgcccgagtgcctgtcccccgccgacgccccctgcgccgtgtcctcctgggcctaccgcctggccgtgcgctcctacgccggctgctcccgcaccaccccccccccccgctgcttcgccgaggcccgcatggagcccgtgcccggcctggcctggctggcctccaccgtgaacctggagttccagcacgcctccccccagcacgccggcctgtacctgtgcgtggtgtacgtggacgaccacatccacgcctggggccacatgaccatctccaccgccgcccagtaccgcaacgccgtggtggagcagcacctgccccagcgccagcccgagcccgtggagcccacccgcccccacgtgcgcgcctaa

SEQ ID NO: 25 - protein, 308 aa, Human alphaherpesvirus 2
KYALADPSLKMADPNRFRGKNLPVLDQLTDPPGVKRVYHIQPSLEDPFQPPSIPITVYYAVLERACRSVLLHAPSEAPQIVRGASDEARKHTYNLTIAWYRMGDNCAIPITVMEYTECPYNKSLGVCPIRTQPRWSYYDSFSAVSEDNLGFLMHAPAFETAGTYLRLVKINDWTEITQFILEHRARASCKYALPLRIPPAACLTSKAYQQGVTVDSIGMLPRFIPENQRTVALYSLKIAGWHGPKPPYTSTLLPPELSDTTNATQPELVPEDPEDSALLEDPAGTVSSQIPPNWHIPSIQDVAPHHAP

SEQ ID NO: 26 - protein, 308 aa, Human alphaherpesvirus 1
KYALADASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAPQIPPNWHIPSIQDAATPYHP

SEQ ID NO: 27 - protein, 286 aa, Human alphaherpesvirus 1
KTSWRRVSVGEDVSLLPAPGPTGRGPTQKLLWAVEPLDGCGPLHPSWVSLMPPKQVPETVVDAACMRAPVPLAMAYAPPAPSATGGLRTDFVWQERAAVVNRSLVIYGVRETDSGLYTLSVGDIKDPARQVASVVLVVQPAPVPTPPPTPADYDEDDNDEGEGEDESLAGTPASGTPRLPPSPAPPRSWPSAPEVSHVRGVTVRMETPEAILFSPGEAFSTNVSIHAIAHDDQTYTMDVVWLRFDVPTSCAEMRIYESCLYHPQLPECLSPADAPCAASTWTSRLA